(19) United States
(12) Patent Application Publication
(10) Pub. No.: US 2022/0143095 A1
(43) Pub. Date: May 12, 2022

(54) NOVEL ANUCLEATED CELLS AND USES THEREOF

(71) Applicant: Platelet Biogenesis, Inc., Watertown, MA (US)

(72) Inventors: Sunita R. Hett, Watertown, MA (US); Karen Weisinger, Watertown, MA (US); Yang Peng, Watertown, MA (US); Kyle P. McLaughlin, Watertown, MA (US); Christian G. Peters, Watertown, MA (US); Andrew R. Zukauskas, Watertown, MA (US); Brenden W. Smith, Watertown, MA (US); Silvia Giannini, Watertown, MA (US); Marcus Lehmann, Watertown, MA (US)

(21) Appl. No.: 17/581,329

(22) Filed: Jan. 21, 2022

Related U.S. Application Data
(63) Continuation of application No. 17/213,552, filed on Mar. 26, 2021.
(60) Provisional application No. 63/105,693, filed on Oct. 26, 2020.
(60) Provisional application No. 63/000,848, filed on Mar. 27, 2020.

(51) Int. Cl. A61K 35/19; A61K 45/06

(52) U.S. Cl. CPC A61K 35/19; A61K 45/06

(57) ABSTRACT

Disclosed herein are non-naturally existing novel platelet variants or platelet like cells (PLCs), extracellular vesicles (EVs), and derivatives thereof. Composition comprising the same and methods for treatment or prevention of diseases or disorders therewith is also disclosed.

### RELATED APPLICATIONS

This application is a continuation application of U.S. application Ser. No. 17/213,552, filed Mar. 26, 2021, which claims the benefit of and priority to U.S. Provisional Patent Application No. 63/000,848, filed Mar. 27, 2020 and U.S. Provisional Patent Application No. 63/105,693, filed Oct. 26, 2020, the entire contents of each of which are herein incorporated by reference.

### FIELD

The present disclosure relates to non-natural anucleated cell population and derivatives thereof. More specifically, the present disclosure is directed to non-natural, novel, platelet-like cells or platelet variants and derivatives thereof, their compositions and uses thereof. This disclosure is also directed to non-natural extracellular vesicles, made in an admixture comprising the platelet-like cells or the platelet variants, their compositions and uses thereof.

### BACKGROUND

Targeting drugs for therapy is always challenging because of occurrence of adverse reactions in patients. For example, the antibody drug conjugation technology (ADC) has long fallen short of its promise. Many experimental ADCs have failed due to the complexity of pairing the right antibody with the appropriate toxic agent. Some were abandoned as too weak; others were too harmful. See https://www.reuters.com/article/us-cancer-adc-focus/drug-developers-take-fresh-aim-at-guided-missile-cancer-drugs-idUSKBN1Z510J. The complexity of antibody-based drug delivery is so immense that Roche CEO Severin Schwan told Reuters. “May be others will be luckier, but we failed to master the complexity.”

Clearly, there is a continuous and evolving need to deliver drugs in a manner that the drugs are easily deployable, are specific to a target, can be delivered at potentially lower doses, are cost effective, and have a long-lasting effect while increasing their efficacy and safety to treat a disease or a disorder.

Bone marrow derived platelets (blood platelets) have not only been recognized as key players in hemostasis and thrombosis but also significantly play an important role in cancer, autoimmune and inflammatory disorders, to name a few. Unfortunately, blood platelets (i.e., bone marrow derived) are only available from human blood and have a high risk of disease transmission. Life, for the blood platelets, is short and full of encumbrances, more so in vitro where the storage of the naturally existing bone marrow derived platelets is difficult and prone to contamination. Moreover, naturally existing bone marrow derived platelets start decaying in about 5 days and are hard to store resulting in loss or lack of supply. Furthermore, there is continuous need to place human volunteers on cell separators to provide the platelets, adding to the cost as well as generating inconsistencies between samples. Pandemics, such as the coronavirus pandemic, causes severe shortage of blood donors so much so that the Red Cross has to face severe platelet shortages due to an unprecedented number of blood drive cancellations during this coronavirus outbreak. For example, a news headline reads, “Blood [platelet] Supply ‘At Risk of Collapse’ as Coronavirus Outbreak Halts Donations” (https://www.nbcboston.com/news/local/baker-state-officials-to-provide-update-on-coronavirus-outbreak/2092556/). In addition to the challenges faced above, extracellular vesicles provide additional challenges due to lack of standardized isolation and purification methods and insufficient clinical grade production.

Hence, there is an unmet and urgent need to have artificially made (i.e., non-natural) platelets or platelet-like cells or platelet variants or derivatives thereof or extracellular vesicles or derivatives thereof that are readily available, are unlimited in supply, consistent in quality, and are free of encumbrances that exist with the naturally existing bone marrow derived platelets or cellular extracellular vesicles and can be used to cure a disease or a disorder.

### SUMMARY

In some embodiments, the disclosure provides non-naturally existing, novel, anucleated platelets or platelet-like cells or platelet variants (collectively referred to as “PLCs” (or in its singular form: “PLC”)) or derivatives thereof that structurally differ from the bone marrow derived platelets. PLCs are artificially generated, biocompatible, and can be made in unlimited supply, in substantially pure form. PLCs are consistent quality and provide a minimal risk of disease transmission.

Advantageously, PLCs or derivatives thereof bind to and clear unwanted proteins or toxins (e.g., antibodies, polypeptides, antigens, diseased proteinaceous molecules, viral or bacterial proteins, biological or chemical toxins) in circulation in a mammalian body (e.g., a human patient). This is accomplished, for example, by the PLCs essentially binding to (i.e., capturing) the unwanted proteins and relocating the captured proteins (e.g., antibodies, polypeptides, antigens, diseased proteinaceous molecules, viral or bacterial proteins) to the liver, where they are degraded or eliminated, thereby freeing a mammal (e.g., a human patient) from the unwanted proteins or toxins. Such unwanted proteins and toxins are, but not limited to, antibodies, polypeptides, antigens, diseased proteinaceous molecules, viral or bacterial toxins or other biological or chemical toxins. In some embodiments, PLCs or derivatives thereof show rapid kinetics of clearance thereby facilitating clearance of proteins or toxins from circulation, for example, by targeting 90% response within 24 hours (for acute) and >90% response rate within 4 weeks, (sustained)>24 weeks (for chronic) diseases. In some embodiments, PLCs or derivatives thereof play a role in inducing liver-mediated antigen tolerization to proteins or toxins. In some embodiments, PLCs or derivatives thereof are negatively charged as compared to donor platelets, which lack the negative charge, which could influence PLCs' interactions with other cells. In some embodiments, PLCs aggregate to form clots and plug injured blood vessel walls. For example, PLCs or derivatives thereof catalyze the activation of the blood clotting cascade as measured by the release of thrombin in plasma. PLCs or derivatives thereof also demonstrate greater adhesivity to collagen and could facilitate PLC-based protein-protein adhesion (e.g., in trauma and healing from trauma related injuries). PLCs or derivatives thereof are essentially allogeneic, are not cancerous, or do not exhibit uncontrolled growth or tumor formation in vivo, thereby facilitating PLC-based therapies. PLCs generally have larger surface area as compared to donor platelets. PLCs are admixtured with extracellular vesicles when prepared in vitro, for example, in a bioreactor or a fluidic device. In some embodiments, the use of the PLCs or derivatives thereof avoid global depletion of normal proteins as only pathogenic proteins (e.g., autoantibodies or viral proteins) are neutralized. PLCs or derivatives thereof also provide the advantage of lacking immunogenicity.

This disclosure also describes genetically engineered PLCs or derivatives thereof or genetically engineered progenitor cells from which the genetically engineered PLC are derived from, their compositions and uses thereof. This disclosure further describes bioconjugates of the PLCs or derivatives thereof, their compositions and uses thereof. Each of these PLCs or derivatives thereof, whether genetically engineered or bioconjugated, are advantageously systemic (i.e., can be distributed into interstitial and intracellular fluids) or migratory, i.e., are easily transported through the bloodstream, further utilizing the rolling, adhesion, and aggregate formation capabilities (i.e., mobility) of the PLCs to travel (i.e., flow through the blood) from a first location, where the PLCs or derivatives thereof are administered, to a second location i.e., an injury or a diseased location, where the PLCs or their bioconjugates or the bioengineered PLCs adhere and aggregate at an injury site or a diseased location to mitigate or eliminate the injury (e.g., bleeding) or the disease (e.g., neoplasm, autoimmune or anti-inflammatory diseases, among others). The PLCs or their bioconjugates or the bioengineered PLCs may also adhere and aggregate at an injury site or a diseased site at the first location (i.e., PLCs or derivatives thereof administered locally to mitigate or eliminate the injury (e.g., bleeding) or the disease (e.g., neoplasm, autoimmune or anti-inflammatory diseases, among others). Another advantage of the PLCs or derivatives thereof or the PLC-conjugates or the bioengineered PLCs is that they can travel through blood flow without inducing immunogenicity, are not cancerous cells; and do not exhibit uncontrolled growth or tumor formation in vivo. PLCs or derivatives thereof also provide the advantage of carrying and delivering to target cells higher drug payloads (e.g., genetically engineered payloads, or conjugated payloads or infused payloads) because of their large surface area as compared to other payload carrying agents such as an antibody in an antibody drug conjugate (ADC).

PLCs or derivatives thereof are variants of bone marrow derived platelets because, essentially, they are man-made by a combination of ex vivo or in vitro processes. The PLC-producing progenitor cells start off as primary expanding/cultured cells, then are reprogrammed to a naive pluripotent state. At this point they become an in vitro clonal cell culture. This clonal population is selected and expanded in vitro, for example, in a bioreactor or a fluidic device, which makes the PLCs as variants of bone morrow derived platelets. The PLCs or derivatives thereof, while unique in their characteristics and functionalities, retain some of functional indices of bone marrow derived platelets, such as but not limited to, comparable or higher levels of the growth factors, receptors or ligands, which makes the PLCs or derivatives thereof uniquely placed to substitute for the donor platelets. Thus, the PLCs or derivatives thereof provide unique utility as a replacement for donor platelets or for treating diseases or disorders where bone marrow platelets play a role but are in short supply or are defective in their physiological properties.

Thus, in some embodiments, the present disclosure provides non-naturally occurring PLCs, a variant of resting reference bone marrow derived platelet cells, structurally comprising greater than an average of 2% CD63 receptors (i.e., CD63^{>average2%}) as compared to the reference resting bone marrow derived platelet cells with less than an average 2% CD63 receptors i.e., (CD63^{<average 2%}).

In some embodiments, the present disclosure provides non-naturally occurring PLCs, a variant of resting reference bone marrow derived platelet cells, structurally comprising less than an average of 96% CD61 receptors (i.e., CD61^{<average96%}) as compared to the reference resting bone marrow derived platelet cells with greater than an average 96% CD61 receptors i.e., (CD61^{>average 96%}).

In some embodiments, the present disclosure provides PLCs, a variant of a resting reference bone marrow derived platelet cells, comprising the structure CD63^{>average2%} CD61^{<average96%} as compared to the reference resting bone marrow derived platelet cells with the structure CD63^{<average 2%} CD61^{>average 96%}.

In some embodiments, the present disclosure provides PLCs, a variant of a resting reference bone marrow derived platelet cells, comprising the structure CD63^{>average2%} TLT-1^{<average 23%} as compared to the reference resting bone marrow derived platelet cells with the structure CD63^{<average 2%} TLT-1^{>average 23%}.

In some embodiments, the present disclosure provides PLCs, a variant of a resting reference bone marrow derived platelet cells, comprising the structure CD63^{>average2%} CD36^{<average80%} as compared to the reference resting bone marrow derived platelet cells with the structure CD63^{<average 2%} CD36^{>average 80%}.

In some embodiments, the present disclosure provides PLCs, a variant of a resting reference bone marrow derived platelet cells, comprising the structure CD63^{>average2%} GPVT^{<average 90%} as compared to the reference resting bone marrow derived platelet cells with the structure CD63^{<average 2%} GPVI^{>average 90%}.

In some embodiments, the present disclosure provides PLCs, a variant of a resting reference bone marrow derived platelet cells, comprising the structure CD63^{>average2}% CD42b^{<average 95%} as compared to the reference resting bone marrow derived platelet cells with the structure CD63^{<average 2%} CD42^{b>average 95%}.

In some embodiments, the present disclosure provides PLCs, a variant of a resting reference bone marrow derived platelet cells, comprising the structure CD63^{>average2%}CD36^{<average 92%} as compared to the reference resting bone marrow derived platelet cells with the structure CD63^{<average 2%} CD36^{>average 92%}.

In some embodiments, the present disclosure provides PLCs, a variant of a resting reference bone marrow derived platelet cells, comprising the structure CD63^{>average2%} GPVI^{average 92%} as compared to the reference resting bone marrow derived platelet cells with the structure CD63^{<average 2%} GPVI^{average 92%}.

In some embodiments, the present disclosure provides PLCs, a variant of a resting reference bone marrow derived platelet cells, comprising the structure CD63^{>average 2%}CD41a^{<average 99%} as compared to the reference resting bone marrow derived platelet cells with the structure CD63^{<average 2%} CD41a^{>average 99%}.

In some embodiments, the present disclosure provides PLCs, a variant of a resting reference bone marrow derived platelet cells, comprising the structure CD63^{>average 2%}CD61^{<average 99%} as compared to the reference resting bone marrow derived platelet cells with the structure CD63^{<average 2%} CD61^{>average 99%}.

In some embodiments, the present disclosure provides PLCs, a variant of a resting reference bone marrow derived platelet cells, comprising the structure CD63^{>average2}% CD42a^{<average 98%} as compared to the reference resting bone marrow derived platelet cells with the structure CD63^{<average 2%} CD42a^{>average 98%}.

In some embodiments, the present disclosure provides PLCs, a variant of a resting reference bone marrow derived platelet cells, comprising the structure CD63^{>average 2%}CD42d^{<average 30%} as compared to the reference resting bone marrow derived platelet cells with the structure CD63^{<average 2%} CD42a^{>average 30%}.

The CD63 structural variant of the resting reference bone marrow derived platelet cells may structurally comprise CD63^{>average 3%}, CD63^{>average 5%}, CD63^{>average 10%}, CD63^{>average 15%}, CD63^{>average 20%}, CD63^{>average 25%}, CD63^{>average 30%}, CD63^{>average 35%}, CD63^{>average 40%}, CD63^{>average 45%}, CD63^{>average 50%}, CD63^{>average 60%}, CD63^{>average 70%}, CD63^{>average 80%} or CD63^{>average 90%} as compared to the reference resting bone marrow derived platelet cells with the structure CD63^{<average 2%}.

The CD63 structural variant of the resting reference bone marrow derived platelet cells may structurally comprise CD63^{>average 3-10%}, CD63^{>average 10-15,%}, CD63^{>average 15-20%}, CD63^{>average 20-25%}, CD63^{>average 25-30%}, CD63^{>average 30-35%}, CD63^{>average 35-40%}, CD63^{>average 40-45%}, CD63^{>average 45-50}, CD63^{>average 50-60%}, CD63^{>average 60-70%}, or CD63^{>average 70-80%}, as compared to the reference resting bone marrow derived platelet cells with the structure CD63^{<average 2-5%.}

The CD63 structural variant of the resting reference bone marrow derived platelet cells may structurally comprise CD36^{<average 1%}, CD36^{<average 2%}, CD36^{<average 3%}, CD36^{<average 4%}, CD36^{<average 5%}, CD36^{<average 6%}, CD36^{<average 7%}, CD36^{<average 8%}, CD36^{<average 9%}, CD36^{<average 10%} or greater than CD36^{<average 10% but essentially less than CD}36^{<average 90%} i.e., as compared to the reference resting bone marrow derived platelet cells with the structure CD36^{>average 90%}.

The CD63 structural variant of the resting reference bone marrow derived platelet cells may structurally comprise CD36^{<average 1-2%}, CD36^{<average 2-3%}, CD36^{<average 3-4%}, CD36^{<average 4-5%}, CD36^{<average 5-6%}, CD36^{<average 6-7%}, CD36^{<average 7-8%}, CD36^{<average 8-9%}, CD36^{<average 9-15%}, CD36^{<average 15-30%} or greater than CD36^{<average 15-30%} but essentially less than CD36^{>average 90%} i.e., as compared to the reference resting bone marrow derived platelet cells with the structure CD36^{>average 90%}.

The CD63 structural variant of the resting reference bone marrow derived platelet cells may structurally comprise CD42b^{<average 10%}, CD42b^{<average 15%}, CD42b^{<average 20%}, CD42b^{<average 25%}, CD42b^{<average 30%}, CD42b^{<average 35%}, CD42b^{<average 40%}, CD42b^{<average 45%} or CD42b^{<average 50%} or greater than CD42b^{<average 50%}, but essentially less than CD42b^{>average 95%} i.e., as compared to the reference resting bone marrow derived platelet cells with the structure CD42b^{>average95%}.

The CD63 structural variant of the resting reference bone marrow derived platelet cells may structurally comprise CD42b^{<average 0-10%}, CD42b^{<average 10-15%}, CD42b^{<average 15-20%}, CD42b^{<average 20-25%}, CD42b^{<average 25-30%}, CD42b^{<average 30-35%}, CD42b^{<average 35-40%}, CD42b^{<average 40-45%}, CD42b^{<average 45-50% or or greater than CD}42b^{<average 45-50%}, but essentially less than CD42b^{>average 95%}. i.e., as compared to the reference resting bone marrow derived platelet cells with the structure CD42b^{>average 95%}.

The CD63 structural variant of the resting reference bone marrow derived platelet cells may structurally comprise CD41a^{<average 60%}, CD41a^{<average 63%}, CD41a^{<average 65%}, CD41a^{<average 68%}, CD41a^{<average 70%}, CD41a^{<average 73%}, CD41a^{<average 76%}, CD41a^{<average 79%}, CD41a^{<average 82%}, CD41a^{<average 85%} or greater than CD41a^{<average 85%} but essentially less than CD41a^{>average 98%} i.e., as compared to the reference resting bone marrow derived platelet cells with the structure CD41a^{>average 98%}.

The CD63 structural variant of the resting reference bone marrow derived platelet cells may structurally comprise CD41a^{<average 55-60%}, CD41a^{<average 60-63%}, CD41a^{<average 63-65%}, CD41a^{<average 65-68%}, CD41a^{<average 68-70%}, CD41a^{<average 70-73%}, CD41a^{<average 73-76%}, CD41a^{<average 76-79%}, CD41a^{<average 79-82%}, CD41a^{<average 82-85%} or greater than CD41a^{<average 82-85%}, but essentially less than CD41a^{>average 98%}, i.e., as compared to the reference resting bone marrow derived platelet cells with the structure CD41a^{>average 98%}.

The CD63 structural variant of the resting reference bone marrow derived platelet cells may structurally comprise GPVI^{<average 1%}, GPVI^{<average 2%}, GPVI^{<average 3%}, GPVI^{<average 4%}, GPVI^{<average 5%}, GPVI^{<average 6%}, GPVI^{<average 7%}, GPVI^{<average 8%}, GPVI^{<average 9%}, GPVI^{<average 10%}, GPVI^{<average 20%}, GPVI^{<average 30%} or greater than GPVI^{<average 30%}, but essentially less than GPVI^{>average 90%}, i.e., as compared to the reference resting bone marrow derived platelet cells with the structure GPVI^{>average 90%}. In some embodiments, the variant comprises less than an average of 5% glycoprotein VI receptor or less i.e., (GPVI^{<average5%} or less) as compared to the reference resting bone marrow derived platelet cells with greater than an average 90% GPVI receptor i.e., (GPVI^{>average 90%}).

In some embodiments, the present disclosure provides non-natural extracellular vesicles (EVs) that are made in vitro as admixtures with the PLCs. Extracellular vesicles (EVs) comprise microvesicles (MVs) or exosomes or a combination thereof, are smaller in size as compared to PLCs, and are biologically active. Each component in the admixture, i.e., PLCs, microvesicles and exosomes can substantially be isolated into individual components from the admixture, for example based on their size. The extracellular vesicles (EVs) function as a transport and delivery system for bioactive molecules, play a role in hemostasis and thrombosis, inflammation, malignancy infection transfer, angiogenesis, and immunity. Thus, in some embodiments, EVs may complement PLCs or their derivatives and their combinational use is an even richer resource for PLC-based therapeutic applications.

In some embodiments, the EVs of the present disclosure comprise exosomes, approximately ranging between 65 nm to about 10 μm in diameter carrying multifarious molecules such as proteins, lipids, and RNAs either on their surface or within their lumen. Exosomes play a role in stimulating tissue regeneration, in many in vitro and in vivo models, demonstrating that they can confer proangiogenic, proliferative, antiapoptotic and anti-inflammatory actions through transporting RNA and protein cargos. Thus, in some embodiments, exosomes make it even a richer resource for PLC-based therapeutic applications.

In some embodiments, the EVs of the present disclosure comprise microvesicles (MVs), approximately ranging between 65 nm to about 10 μm in diameter, carrying multifarious molecules such as proteins, lipids, and RNAs either on their surface or within their lumen. MVs play a role in stimulating tissue regeneration, in many in vitro and in vivo models, demonstrating that they can confer proangiogenic, proliferative, antiapoptotic and anti-inflammatory actions through transporting RNA and protein cargos. Thus, in some embodiments, MVs make it even a richer resource for PLC-based therapeutic applications.

In a migratory role, PLCs and/or EVs or derivatives thereof travel to a diseased location where there is at least one tumor or tumor cells, the non-naturally occurring PLCs and/or EVs, or their derivatives, having the ability to travel through and around tumors, surround the tumor, i.e., aggregate around the tumor to deliver cytotoxic agents that kill the tumor cells. Further, the PLCs or the EVs, or derivatives thereof, tend to interact with metastasizing cancer cells, PLCs and/or EVs, or derivatives thereof, have the capacity to track at least one infiltrating tumor cell, thereby delivering cytotoxic agents to the metastasizing cancer cell, thereby inhibiting tumor metastasis. Tumor cells can also be treated after tumor metastasis has occurred.

In some embodiments, the megakaryocytic progenitors, megakaryocytes, proplatelets, preplatelets derived from induced pluripotent stem cells (iPSCs), which produce the platelet like cells (PLCs) and EVs (i.e., microvesicles or exosomes or a combination thereof), prior to being passaged through a bioreactor or a fluidic device, can be genetically engineered to express a nucleic acid encoding a protein of interest (including a polypeptide, peptide or an antigen of interest). In some embodiments, PLC and/or EVs can be genetically engineered once such cells were subjected to a passage through the bioreactor or a fluidic device. Thus, in some embodiments, genetic modifications can take place at the stem cell level, in megakaryocytes or in some embodiments in the PLCs and/or the EVs or at any other level during the generation of PLCs and/or the EVs that accompany the PLC and/or EV production. Genetic engineering of megakaryocytes or megakaryocytic progenitors differentiated from a genetically engineered human pluripotent stem cells (hPSCs) cell or cell lines, where the genetic manipulation leads to megakaryocytes or megakaryocytic progenitor cells to express a protein or a polypeptide of interest are also contemplated by the disclosure. In some embodiments, the PLCs and/or EVs or derivatives thereof, differentiated from the genetically engineered progenitor cells (e.g., megakaryocytes or megakaryocytic progenitor cells), deliver a protein of interest systemically or at first diseased location, generally the site of a disease where the PLCs and/or the EVs are administered, or to a second diseased location, different from the site where the PLCs and/or EVs or derivatives thereof are administered. Non-limiting examples of such genetically engineered induced pluripotent stem cells or PSC-derived megakaryocytes that produce the PLCs and/or EVs (i.e., engineered PLCs/EVs or derivatives thereof) are also disclosed herein.

Exemplary vectors that can be used to genetically engineer the iPSCs or megakaryocytes that produce the PLCs and/or EVs or derivatives of the present disclosure, such that the PLCs and/or the EVs produce a genetic product (i.e., derivatives of PLCs and/or EVs) intended to be produced in the PLCs and/or the EVs (e.g., IL-12, CTLA4 or HGF or other growth factor or cytokine, ligand or a receptor or an antigen, or an antibody or a fragment thereof) include, but are not limited to, plasmids, retroviral vectors, lentiviral vectors, adenovirus vectors, adeno-associated virus (AAV) vectors, a herpes simplex virus vectors, poxvirus vectors, or baculoviral vectors. The vector comprising the nucleic acid molecule of interest may be delivered to the cell (e.g., iPS cell, megakaryocytic progenitor, or megakaryocyte) via any method known in the art, including but not limited to transduction, transfection, infection, and electroporation.

In some embodiments, the present disclosure provides PLCs and/or the EVs or derivatives thereof that take the advantage of their cargo carrying capacity. The large surface area of the PLCs and their membrane flexibility permits the PLCs or derivatives thereof to carry a much larger drug payload to deliver to their targets as compared to, for example, drug antibody conjugates. Added surface area is provided by the exosomes or microvesicles. Cargos for delivery to a diseased location may include cytotoxic agents, such as but not limited to nucleic acids, proteins or polypeptides, small molecules or conjugates thereof or a combination thereof. Cytotoxic agents are disclosed elsewhere in the application. In some embodiments, cargos may also include a therapeutic RNA or DNA for delivery to a target cell at a diseased location. In some embodiments, cargos may also include a combination of 2 or more payloads (e.g., two different proteins, or RNAs or DNAs or antibodies or fragments thereof or two different drugs) for delivery to a diseased location. In some embodiments, cargos may be inclusive of endogenous molecules in the PLCs' or exosomes' or microvesicles' cargo space and one or more of exogenous molecules imbibed or diffused into the same cargo space. Exogenous molecules could be an antibody or a fragment thereof (e.g., a human or a humanized antibody) that is imbibed or diffused into the PLCs and/or EVs (e.g., microvesicles or exosomes) or could be a therapeutic small molecule (maytansinoid, checkpoint inhibitors) or a protein (e.g., antibodies or fragments thereof, polypeptide, IL-12, factor VIIa or HGF, among others) or a nucleic acid (e.g., siRNA). The advantage of this approach is it provides improved drug efficacy with reduced systemic toxicity.

In some embodiments, the present disclosure provides bioconjugates comprising PLCs and/or EVs that are conjugated to a cytotoxic agent such as an antibody (e.g., a human or a humanized antibody) or a fragment thereof, or a drug (e.g., a chemotherapeutic agent), or a growth inhibitory agent, a toxin (e.g., an enzymatically active toxin of bacterial, fungal, plant, or animal origin, or fragments thereof), or a radioactive isotope (i.e., a radioconjugate). In some embodiments, the present disclosure provides methods of using the PLCs and/or EVs, which are conjugated to a cytotoxic agent to treat or ameliorate a disease or a disorder.

When a linker is used in bioconjugation of the PLCs and/or EVs or derivatives thereof, the PLC or EV bioconjugates have the formula (A)-(L)-(C), where: (A) is non-naturally occurring PLCs and/or EVs described herein; (L) is a linker; and (C) is a cytotoxic agent; and where the linker (L) links (A) to (C). It is understood that when a linker links a PLC or EV or a derivative thereof to a cytotoxic agent, one or more amino acids, for example, on a PLC receptor (e.g., CD63) may be utilized for linking the cytotoxic agent to the PLCs. For example, one or more amino acids on the CD63 receptor in PLCs may be used to attach one or more linkers to the PLC's-CD63 receptor, which may then be conjugated to a cytotoxic agent via the attached linkers. Likewise, EVs may be conjugated via one or more EV-based receptors (e.g., CD9 receptor) and, in the above formula, A is a non-naturally occurring EV described herein. Alternatively, the linkers are first conjugated to the cytotoxic agent and then attached to one or more amino acid residues in the PLC-CD63 receptor or the EV-CD9 receptor or to both. Typically, this configuration is represented as PLC-CD63-Linker-C or EV-CD9-Linker-C, where the amino acid residues (e.g., cysteine, lysine etc.) in the PLC CD63 receptor or the EV CD9 receptor is linked via a linker to a cytotoxic agent. The cytotoxic agent could be an antibody or a fragment thereof, a protein or a polypeptide, a prodrug or a drug and are described in detail elsewhere in the application.

When an antibody is selected for conjugation to the PLCs and/or EV via a linker, the shared amino acid between an antibody and a receptor or different amino acids on an antibody and a receptor can be utilized for their conjugation. For example, Lysine residues shared between a PLC/EV receptor and an antibody can be used to covalently attach to the PLC or EV receptor to the antibody in a formula PLC-CD63-Lysine-Linker-Lysine-Antibody. In this example, the lysine residue in the PLC's CD63 receptor (PLC-CD63-Lysine) and the lysine residues in the antibody (i.e., Lysine-Antibody) are modified to covalently attach PLCs to the antibody via the linker. Alternatively, different amino acid residues in a PLC receptor and an antibody can be utilized for their conjugation. For example, Lysine residues in a PLC's/EV's receptor is used whereas a cysteine residue in an antibody is used to attach to a linker of the formula PLC-Lysine-Linker-cysteine-Antibody. Any antibody can be attached via a linker to the PLCs of the present disclosure. Likewise, the lysine or cystine amino acids on one of more EV receptors (e.g., CD9) can be manipulated with linkers to have a formula: EV-CD9-Lysine-Linker-Lysine-Antibody or EV-CD9-Lysine-Linker-cysteine-Antibody configuration.

When the cytotoxic agent is a prodrug or a drug, one or more receptors on the PLCs and/or EVs or derivatives thereof can be modified to conjugate to a linker in a manner discussed in the foregoing. The linker is then conjugated to a prodrug or a drug moiety via well-established techniques.

In some embodiments, the present disclosure provides a pharmaceutical composition comprising the non-naturally occurring PLCs and/or EVs or derivatives thereof and a pharmaceutically acceptable carrier. In some embodiments, the pharmaceutical composition comprises one or more of a second therapeutic agent.

In some embodiments, the PLCs and/or EVs or derivatives thereof may imbibe into the PLCs' or EVs' secretory granules, such as but not limited to—α-granules or dense granules, an antibody, a growth factor, a ligand, an antigen, or a nucleic acid (e.g., siRNA), which can then be transported and released with such granules.

In some embodiments, the present disclosure provides a method of treating a patient suffering from a disease or a disorder (e.g., immunoinflammatory disorder, a metabolic disorder, a neoplastic disorder, an autoimmune disorder, viral or bacterial-induced disorder) the method comprising administering to the patients the PLCs and/or EVs or derivatives thereof of the present disclosure thereby causing amelioration of or treatment of the disease or the disorder. The PLCs and/or EVs or derivatives thereof may be administered together or separately.

In some embodiments, the present disclosure provides a method of treating a patient suffering from a disorder in which the disorder is inclusive of a ligand, receptor or an antigen that has affinity for a receptor or a ligand on the PLCs and/or EVs or derivatives thereof. In some embodiments, the PLCs and/or the EVs or derivatives thereof can bind to a ligand or an antigen of interest on a diseased cell thereby ameliorating or treating a disorder by blocking the activity of the ligand or the antigen, the method comprising administering to the patients the PLCs and/or EVs or derivatives thereof of the present disclosure expressing a receptor that will specifically bind to the ligand or antigen with relatively high affinity, the receptor-ligand or receptor-antigen interaction causing removal or degradation of the toxic molecule thereby amelioration of or elimination of the disorder. In some embodiments, the PLCs and/or the EVs or derivatives thereof can bind to a receptor of interest on a diseased cell thereby ameliorating or treating a disorder by blocking the activity of the receptor, the method comprising administering to the patients the PLCs and/or EVs or derivatives thereof of the present disclosure expressing a ligand that will specifically bind to the receptor on the diseased cell with relatively high affinity, the receptor-ligand interaction causing amelioration of or elimination of the disorder. In some embodiments, the PLC and/or EV receptors are conjugated to a cytotoxic agent, which are then delivered to a diseased cell bearing the ligand of interest. In some embodiments, cytotoxic agents are carried as a deliverable cargo by the PLCs and/or EVs, which are then delivered to a diseased cell bearing the ligand/receptor of interest. In some embodiments, the genetically engineered PLCs and/or EVs deliver a cytotoxic agent to a diseased cell bearing the ligand/receptor of interest. In some embodiments, the therapeutic effect is to ameliorate or treat a disease or a disorder caused by the diseased cells bearing the ligand/receptor of interest.

In some embodiments, the present disclosure provides a therapeutical method for treating a mammal having a tumor in a tumor microenvironment, the method comprising targeting donor platelets in a cancer microenvironment with PLCs and/or EVs or derivatives thereof that act as decoys. In some embodiments, the PLCs and/or EVs or a combination thereof are anticipated to carry drug payloads (e.g., antibodies, siRNA, growth factors or a combination thereof), which are then contacted by a tumor metastasizing cells thinking it to be donor platelet (donor platelets facilitate tumor growth in a tumor microenvironment) and the drug on the PLCs and/or EVs or derivatives thereof target the tumors particularly to destroy or inhibit cancer cells from metastasizing in a tumor microenvironment). The method can comprise administering to a mammal a therapeutically effective amount of PLCs and/or EVs or derivatives thereof programmed to act as decoys, thereby effectively treating the tumor or at least preventing the tumor from spreading. In some embodiments, the PLCs and/or EVs or derivatives thereof carry drug payloads. For example, PLCs and/or EVs or derivatives thereof or a combination thereof are conjugated to a cytotoxic agent or the PLCs and/or EVs or a combination thereof are conjugated to a growth inhibitory agent or the PLCs and/or EVs or a combination thereof are genetically engineered PLCs and/or EVs to deliver a target molecule (e.g., siRNA) or a combination thereof which produce a cytotoxic agent or a growth inhibitory agent to effectively target tumor cells from spreading.

In some embodiments, the PLCs and/or EVs or derivatives thereof carry growth factors or cytokines for tissue regeneration. In some embodiments, the PLCs and/or EVs or derivatives thereof deliver, proteins expressed in the PLC and/or EV granules (e.g., alpha-granules), or deliver proteins expressed on their cell surface or deliver proteins expressed in their transmembrane domains, or deliver proteins packaged in PLCs and/or EVs (e.g., microvesicles or exosomes).

In some embodiments, the present disclosure provides a diagnostic reagent comprising the non-naturally occurring PLCs and/or EVs or derivatives thereof where the PLC and/or EV receptors or ligands or the cell surface of the PLCs and/or EVs are labeled. The label is selected from the group consisting of a radiolabel, a fluorophore, a chromophore, an imaging agent and a metal ion. Labelling techniques are well known to one of skill in the art.

In some embodiments, the present disclosure provides a kit comprising the PLCs and/or EVs or derivatives thereof of the present disclosure described herein. Here, the PLCs and/or EVs are engineered to recognize one or more viral receptors or protein for an early diagnostic of viral infections such a coronavirus or Ebola virus, or any virus if the PLCs or derivatized PLCs recognize such viral receptors or proteins.

### BRIEF DESCRIPTION OF FIGURES

The presently disclosed embodiments will be further explained with reference to the attached drawings, in which:

FIG. 1 is an exemplary example illustrating the structural makeup of the PLCs, variants of donor platelets.

FIGS. 2A-2E are exemplary illustrations, which structurally distinguish the PLCs from bone marrow derived platelets. FIG. 2A shows CD63 and PAC1 structural differences between donor platelets and PLCs. FIG. 2B shows CD42a, CD42b and CD36 structural differences between donor platelets and PLCs. FIG. 2C shows CD61, CD41a and CD42a structural differences between donor platelets and PLCs. FIG. 2D shows CD61 and GPVI structural differences between donor platelets and PLCs. FIG. 2E shows CD61, CD41a and PAC1 structural differences between donor platelets and PLCs, as shown by flow cytometric analysis.

FIGS. 3A through 3C show morphological structure of PLCs vs. donor platelets. Non-naturally occurring PLCs are shown in FIG. 3A, which could comprise of extracellular vesicles (e.g., exosomes) as admixtures (FIG. 3C). In comparison to Figure PLCs shown in FIG. 3A, FIG. 3B shows donor platelets. FIG. 3D illustrates that PLCs are rich in several growth factors, which are in greater or comparable quantities as compared with donor platelets (dPLT).

FIGS. 4A-4E are illustrative examples of unique functionalities of the PLCs. FIG. 4A is a thrombin generation assay. FIG. 4B is a velocity index study of the PLCs which shows PLCs have greater adhesivity to collagen as compared to donor platelets. FIG. 4C shows adhesion velocity of the PLCs as compared to donor platelets. FIG. 4D exemplifies clearance kinetics of the PLCs as compared to donor platelets. FIG. 4E is an illustration of an experimental design to show how the quantification CD41/CD61 antibodies was performed (top) and shows PLCs are liver-bound (bottom).

FIGS. 5A-5C are illustrative examples of a PLC-bioconjugate of the present disclosure and assays therefrom. FIG. 5A is a schematic example for conjugating PLC via a linker, in this case to an antibody. FIGS. 5B-5C exemplify conjugating to PLCs. In this case, ipilimumab to the PLCs (FIG. 5B) and assessment thereof (FIG. 5C).

FIGS. 6A-6C further illustrate PLC-antibody conjugates and activity thereof. FIG. 6A is another schematic example for conjugating PLC via a linker to an antibody and functional evaluation thereof (FIG. 6B). FIG. 6C is an image of anti-CTLA4 mAb (broken arrow) that was chemically conjugated to the surface of the PLC.

FIGS. 7A-7B are illustrative examples of drugs, in this case, doxorubicin, being imbibed by the PLCs and activities thereof.

FIG. 8A is a schematic illustration of isolation of bioreactor-derived extracellular vesicles. FIG. 8B shows pelleted EVs.

FIGS. 9A-9E show morphology and size characterization of bioreactor-derived EVs.

FIG. 9A shows PLCs (left panel), EVs (middle panel) and exosomes as an admixture in the EVs (right panel). FIGS. 9B through 9E show particle concentration versus size for the EVs.

FIGS. 10A-10C show characterization of bioreactor-derived EV surface markers (FIG. 10A) and exosome markers (FIGS. 10B and 10C).

FIGS. 11A-11B show further characterization of bioreactor-derived EV surface markers CD42b, CD61 via FACS analysis.

FIGS. 12A-12B show bioreactor-derived EV uptake by HepG2 cells.

FIGS. 13A-13B show bioreactor-derived EV uptake by HCT116 cells.

FIG. 14A shows images of bioreactor-derived EV uptake by HepG2 cells. FIG. 14B shows images of bioreactor-derived EV uptake by HCT116 cells. FIG. 14C is a negative control for the bioreactor-derived EV uptake by HepG2 cells (negative control). FIG. 14D is a negative control for the bioreactor-derived EV uptake by HCT116 cells (negative control).

FIGS. 15A-15B show mechanisms of EV uptake by HepG2 cells in presence of or in absence of inhibitors.

FIGS. 16A-16B show mechanisms of EV uptake by HCT116 cells in presence of or in absence of inhibitors.

FIGS. 17A-17B illustrate an example of biological products that can be delivered externally by the PLCs (e.g., secreted proteins) and internally by the exosomes (e.g., siRNA) into a target cell. FIG. 17A exemplifies an uptake of exosomes by HepG2 cells (hence the feasibility of the delivery of siRNA, for example as shown in FIG. 17B). Exogenous expression of a protein in the PLCs is shown elsewhere.

FIGS. 18A-18B show optimized isolated bioreactor-derived extracellular vesicles (EVs) and their surface marker characterization (FIG. 18A). FIG. 18B shows labeling and uptake of EVs with examples of uptake in HepG2 (Human liver cancer cell line) and HCT-116 (Human colon cancer cell line), respectively.

FIGS. 19A-19B show that the expression of IL-12 protein is upregulated in engineered EVs derived from genetically engineered IL-12 PLC/EV producing progenitor cells that also produce engineered PLCs (ePLCs).

FIGS. 20A-20B show that siRNA can be loaded into PLC-EVs exogenously and delivered to HepG2 cells.

FIGS. 21A-21E show that EVs are capable of delivering cargos to target cells. FIGS. 21A through 21E are Imaging of co-localization of EVs and siRNAs in the HepG2 cells indicating that EV-loaded siRNAs can be efficiently taken in by HepG2 cells.

FIG. 22 shows that siRNAs are biologically functional after delivering to HepG2 cells.

FIG. 23 shows that PLC-EVs were lacking in prostaglandin F2 receptor inhibitor (PTGFRN) expression.

FIG. 24 shows a schematic model of knocking in genes to generate engineered PLCs. With FVII (solid arrow) as an example.

FIG. 25A is an exemplary illustration of a lentivirus vector used for generating engineered PLC (ePLCs). FIGS. 25B-25D show lentiviral transduced Zs green were expressed in pluripotent stem cells and MKs and PLCs derived therefrom.

FIG. 26 is another example of an expression vector for making ePLCs, in this case ePLCs expressing HGF and IL-12.

FIGS. 27A-27C are an example showing genetically engineered PLC (ePLCs) can express a protein of interest, in this case expressing HGF. HGF protein as measured by ELISA is increased in HGF expressing single cell PLC clone G8 generated from transduced iPSC populations (FIG. 27A). A cellular activity assay confirms active HGF protein expressed from clone G8 (FIG. 27B). Expression of HGF in ePLC (HGF-PLCs) in comparison to donor platelets and untransduced PLC is shown in FIG. 27C.

FIGS. 28A-28D are another example of ePLCs expressing an exogenous gene, in this case IL-12. IL-12 protein is elevated in the IL-12 transduced cell population compared to PBG1 control (untransduced) (FIG. 28A). IL-12 protein levels in single cell derived clones shows high IL-12 expression from clone H2 (FIG. 28B). FIGS. 28C-28D shows IL-12 protein levels in the H2 clone differentiated to MLC and PLC.

FIG. 29 shows engineered iPSC are capable of expressing combination of therapeutic payload(s) from the same cell as exemplified by the co-expression of IL-12 and PD-1, which inherently are delivered by the ePLCs and eEVS, as they are derived therefrom.

FIGS. 30A-30B show non-engineered PLCs reduce liver fibrosis in mouse disease model.

FIG. 31 shows high levels of HGF are expressed in ePLC.

FIGS. 32A-32D show HGF-ePLC dosed in liver fibrosis mice show HGF protein and ePLC in liver. FIG. 32A shows an Experimental Plan for in vivo localization. FIG. 32B shows examining circulating PLCs in mice. FIGS. 32C and 32D show fluorescence staining of livers removed from treated fibrotic mice for HGF (FIG. 32C) and CD61 (FIG. 32D).

FIGS. 33A-33F show high levels of FVII are expressed in ePLC. FIG. 33A illustrates and example of another lentiviral vector used in this study. FIG. 33B shows examples of FVIIa constructs. FIG. 33C shows an image analysis of FVIIa expression. FIG. 33D is an example of protein activity assay, exemplified with FVIIa activity assay. FIG. 33E shows an example of ELISA study with proteins, exemplified with FVII ELISA. FIG. 33F is an example of western blot analysis performed to determine protein expression, exemplified with the expression of FVII.

FIGS. 34A-34B(i-v) show examples of some of the genes that can be genetically engineered into the PLCs, the expressions of which can be characterized in the same manner as described in FIGS. 33A through 33F.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

In the following, the elements of the present disclosure will be described. These elements are listed with specific embodiments; however, it should be understood that they may be combined in any manner and in any number to create additional embodiments. The variously described examples and preferred embodiments should not be construed to limit the present disclosure to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991), Merriam-Webster Medical Dictionary, available online. As used herein, the following terms have the meanings ascribed to them below, unless specified otherwise.

The terms “agent,” “therapeutic agent,” “therapeutic composition,” “drug,” or “therapeutic” can be used interchangeably and are meant to include any small molecule chemical compound, antibody, nucleic acid molecule, or polypeptide, or fragments thereof.

The term “antibody,” as used herein, refers to an immunoglobulin molecule which specifically binds with an antigen. The term “antibody fragment” refers to a portion of an intact antibody and refers to the antigenic determining variable regions of an intact antibody.

By “alteration” or “change” is meant an increase or decrease. An alteration may be by as little as 1%, 2%, 3%, 4%, 5%, 10%, 20%, 30%, or by 40%, 50%, 60%, or even by as much as 70%, 75%, 80%, 90%, or 100%.

By “biologic sample” is meant any tissue, cell, fluid, or other material derived from an organism.

By “capture reagent” is meant a reagent that specifically binds a nucleic acid molecule or polypeptide to select or isolate the nucleic acid molecule or polypeptide.

By “cellular composition” is meant any composition comprising one or more isolated cells.

By “cell survival” is meant cell viability.

By “effective amount” is meant the amount of an agent required to produce an intended effect.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Non-natural” as used herein refers to manufactured, created, or constructed by human beings, artificial, or mimicking something that exists in nature.

The term “structure” as used herein refers to receptor distributions which are unique to the artificially produced (non-natural) platelet like cells. The PLCs are structurally distinguished from the bone marrow derived platelets based on the non-natural distribution of certain receptors on the PLCs. As an example, resting PLC, rich in CD63 distribution, are structurally distinguished from resting bone marrow derived platelets having little or no CD63 receptor. In a simplified explanation, structurally, PLCs having 60%, CD63 receptors are structurally represented as CD63^{60%} whereas bone marrow derived platelets have 2%, CD63 receptors are represented as CD63^{2%}. In some embodiments, structurally PLCs having an average of 66%, CD61 receptor is represented as CD61^{average66%} whereas bone marrow derived platelets have an average of 96%, CD61 receptors is represented as CD61^{average96%}. Various structural differences between the PLCs and donor platelets are described throughout the application and are covered by this definition. Simply stated the structurally differentiated PLCs are unique and not found in nature. As used herein the term structure is also used in context of cell size, cell dimension, surface area or volume. One of skill in the art would understand the specification discloses structures of non-natural PLCs that distinguishes the PLCs from bone marrow derived natural platelets.

“Resting stage” or “resting” refers to a stage in which cells are circulating in blood vessels without forming interactions with non-activated vascular endothelium under normal physiologic conditions.

“Derivatives”, as used herein, refer to genetically engineered PLCs or extracellular vesicles or a combination thereof for therapeutic use, inclusive of PLC precursor cells (e.g., pluripotent stem cells genetically engineered in a manner such that that the PLCs or extracellular vesicles produced by these PLC/EV precursor cells produce a molecule of interest in the PLCs or extracellular vesicles or in both, and in any other modification of described herein. Derivatives are also inclusive of bioconjugates of PLCs and extracellular vesicles or bioconjugates of genetically engineered PLCs and extracellular vesicles. Derivatives are also inclusive of cargo carrying PLCs and extracellular vesicles or cargo carrying genetically engineered PLCs and extracellular vesicles. Here. For example, the PLCs or extracellular vesicles can be first subjected to genetic engineering, then their cargo carrying capacity is utilized. In other words, the term derivative is inclusive of any modification, genetic, chemical or a combination thereof or otherwise of the PLCs, genetically engineered PLC, extracellular vesicles or genetically engineered extracellular vesicles.

The term “extracellular vesicles (EVs or EV)” as used herein collectively refers to microvesicles and exosomes and generally are very small (generally around 1 micron or less in diameter; microvesicles, generally about 200-1500 nm or less in diameter; exosomes generally about 20-200 nm or less in diameter) phospholipid vesicle shed from a megakaryocyte or other cell. Extracellular vesicles (EV) may contain or may transport materials such as but not limited to nucleic acids (e.g., siRNA), growth factors, proteins or exogenous genetic materials (e.g., for gene therapy) and express the extracellular markers of their parental cells. Megakaryocyte-derived extracellular vesicles (EV) may have a role in multiple pathways, including hemostasis and inflammation, and in treating various disorders, such as but not limited to, malignancies (e.g., neoplasia), Alzheimer, and tumor progression and development.

As used herein “progenitor cells” refers to IPSC-derived cells, such as preMKs, MKs, proplatelets, preplatelets. It is also inclusive of “pluripotent stem cells”, which includes embryonic stem cells, embryo-derived stem cells, and induced pluripotent stem cells and other stem cells having the capacity to form cells from all three germ layers of the body, regardless of the method by which the pluripotent stem cells are derived. Pluripotent stem cells are defined functionally as stem cells that can have one or more of the following characteristics: (a) be capable of inducing teratomas when transplanted in immunodeficient (SCID) mice; (b) capable of differentiating to cell types of all three germ layers (e.g., can differentiate to ectodermal, mesodermal, and endodermal cell types); or (c) express one or more markers of embryonic stem cells (e.g., express October 4, alkaline phosphatase. SSEA-3 surface antigen, SSEA-4 surface antigen, SSEA-5 surface antigen, Nanog, TRA-1-60, TRA-1-81, SOX2, REX1. Progenitor cells also include “megakaryocytic progenitor” (preMK), which refers to a mononuclear hematopoietic cell that is committed to the megakaryocyte lineage and is a precursor to mature megakaryocytes. Megakaryocytic progenitors are normally found in (but not limited to) bone marrow and other hematopoietic locations, but can also be generated from pluripotent stem cells, such as by further differentiation of hemogenic endothelial cells that were themselves derived from pluripotent stem cells.

“Agonist Activated” cell receptor or ligand activation induced by a receptor specific agonist. Agonists activate cells by binding to their respective receptors or ligands on a cell.

“Donor platelets” refer to physiologically generated platelets in a mammalian (e.g., human) body, for example bone marrow derived platelets.

“PLC” or “PLCs” or artificial platelets as interchangeably used herein, refer to non-naturally existing, novel, anucleated platelets or platelet-like cells that structurally differ from naturally existing bone marrow derived platelets (i.e., natural counterpart). PLC or PLCs are also inclusive of platelet variants, defined elsewhere.

“Variant” or “Variants” as interchangeably used herein refers to manifesting structural variety, structural deviation, or structural differences between PLCs and donor platelets. As non-limited examples, variant comprises greater than an average of 2%, CD63 receptors (i.e., CD63^{>average2%}) as compared to the reference resting bone marrow derived platelet cells with less than an average 2%, CD63 receptor i.e., (CD63^{<average 2%}). In some embodiments, a variant comprises less than 10% on an average of CD36 receptor (i.e., CD36^{<average80%}) as compared to the reference resting bone marrow derived platelet cells with greater than an average 80%, CD36 receptor i.e., (CD36^{>average 80%}); or a variant comprising less than an average of 95%, CD42b receptor (i.e., CD42b^{<average 95%}) as compared to the reference resting bone marrow derived platelet cells with greater than an average 95%, CD42b receptor i.e., (CD42b^{>average 95%}); or a variant comprising less than an average of 90% glycoprotein VI receptor or less i.e., (GPVI^{<average90%}) as compared to the reference resting bone marrow derived platelet cells with greater than an average 90% GPVI receptor i.e., (GPVI^{>average 90%}). The term variant is also inclusive of a structural makeup of the PLCs that is comparable to the structural make-up of naturally existing bone marrow derived platelets, either in resting or in their activated stages. For example, the PLCs and the donor platelets may have m % CD36, or n % CD42a, or o % CD42a-b-d, or p % CD61, or q % CD62p, or x % CD63 receptors, where the m %, n %, o %, p %, q % x % are the same (i.e., have equal values) between the PLCs and the and bone marrow derived platelets. In other words, structurally PLCs may be identical to donor platelets, yet manifest the advantages of the PLC variants disclosed herein in this application.

As used herein, the terms “treat,” treating,” “treatment,” and the like refer to reducing or ameliorating a disorder and/or symptom associated therewith. It will be appreciated that, although not precluded, treating a disorder or condition does not require that the disorder, condition or symptoms associated therewith be eliminated.

By, “comprises,” “comprising,” “containing” and “having” and the like can have the meaning ascribed to them in U.S. patent law and can mean “includes,” “including,” and the like (e.g., a composition “comprising” X may consist exclusively of X or may include something additional, e.g., X+Y); “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

Unless specifically stated or obvious from context, as used herein, the term “or” is understood to be inclusive. Unless specifically stated or obvious from context, as used herein, the terms “a”, “an”, and “the” are understood to be singular or plural.

The word “substantially” does not exclude “completely” e.g., a composition which is “substantially free” from Y may be completely free from Y. Where necessary, the word “substantially” may be omitted from the definition of the disclosure.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

“Average” as used herein is a number expressing the central or typical value in a set of data, in particular the mode, median, or (most commonly) the mean, which is calculated by dividing the sum of the values in the set by their number. It also refers to a single value (such as a mean, mode, or median) that summarizes or represents the general significance of a set of unequal values.

“Linker” as used herein, refers to “bifunctional crosslinking agent,” “bifunctional linker” or “crosslinking agents” refers to modifying agents that possess two reactive groups; one of which is capable of reacting with a cell binding agent while the other one reacts with the cytotoxic compound to link the two moieties together. Such bifunctional crosslinkers are well known in the art. A “linker,” “linker moiety,” or “linking group” as defined herein also refers to a moiety that connects two groups, such as a cell binding agent and a cytotoxic compound, together. Typically, the linker is substantially inert under conditions for which the two groups it is connecting are linked. A bifunctional crosslinking agent may comprise two reactive groups, one at each ends of a linker moiety, such that one reactive group can be first reacted with the cytotoxic compound to provide a compound bearing the linker moiety and a second reactive group, which can then react with a cell binding agent. Alternatively, one end of the bifunctional crosslinking agent can be first reacted with the cell binding agent to provide a cell binding agent bearing a linker moiety and a second reactive group, which can then react with a cytotoxic compound. The linking moiety may contain a chemical bond that allows for the release of the cytotoxic moiety at a specific site. Suitable chemical bonds are well known in the art and include disulfide bonds, thioether bonds, acid labile bonds, photolabile bonds, peptidase labile bonds and esterase labile bonds (see for example U.S. Pat. Nos. 5,208,020; 5,475,092; 6,441,163; 6,716,821; 6,913,748; 7,276,497; 7,276,499; 7,368,565; 7,388,026 and 7,414,073). Preferred are disulfide bonds, thioether and peptidase labile bonds. Other linkers that can be used in the present disclosure include non-cleavable linkers, such as those described in are described in detail in U.S. publication number 20050169933 or charged linkers or hydrophilic linkers and are described in US 2009/0274713, US 2010/01293140 and WO 2009/134976, each of which is expressly incorporated herein by reference, each of which is expressly incorporated herein by reference. A “Linker” (L) is a bifunctional or multifunctional moiety that can be used to link one or more drug moieties (D) to a PLC to form an PLC bioconjugate of Formula PLC-L-C. In some embodiments, PLC-drug conjugates can be prepared using a Linker having reactive functionalities for covalently attaching to the drug and to the PLC. For example, in some embodiments, a cysteine thiol of a PLC receptor (e.g., CD68, CD36, CD42b, lactadherin, etc.) can form a bond with a reactive functional group of a linker or a drug-linker intermediate to make a PLC bioconjugate.

“Cleavable” as used herein refers to a linker or linker component that connects two moieties by covalent connections but breaks down to sever the covalent connection between the moieties under physiologically relevant conditions, typically a cleavable linker is severed in vivo more rapidly in an intracellular environment than when outside a cell, causing release of the payload to preferentially occur inside a targeted cell. Cleavage may be enzymatic or non-enzymatic, but generally releases a payload from an antibody without degrading the antibody. Cleavage may leave some portion of a linker or linker component attached to the payload, or it may release the payload without any residual part or component of the linker.

“Non-cleavable” as used herein refers to a linker or linker component that is not especially susceptible to breaking down under physiological conditions, e.g., it is at least as stable as the PLC receptor proteins. Such linkers are sometimes referred to as “stable”, meaning they are sufficiently resistant to degradation to keep the payload connected to the PLC receptor until PLC is itself at least partially degraded, i.e., the degradation of PLC precedes cleavage of the linker in vivo. Degradation of the PLC portion an ADC having a stable or non-cleavable linker may leave some or all the linker, and one or more amino acid groups from a PLC, attached to the payload or drug moiety that is delivered in vivo.

“Bioconjugation” refers to conjugating PLCs to a cytotoxic agent with or without the use of a linker. Bioconjugation techniques are well known to one of skill and the art and can be found, for example, in Bioconjugate Techniques, 3rd Edition (2013) by Greg T. Hermanson (ISBN 978-0-12-382239-0: Academic Press). “Bioconjugate Techniques.” besides being a complete textbook and protocols-manual for biomolecular crosslinking, is also an exhaustive and robust reference for conjugation strategies.

The term “amino acid” refers to naturally occurring and synthetic amino acids, as well as amino acid analogs and amino acid mimetics that function in a manner like the naturally occurring amino acids. Naturally occurring amino acids are those encoded by the genetic code, as well as those amino acids that are later modified, e.g., hydroxyproline, gamma-carboxyglutamate, selinocystiene and O-phosphoserine. Amino acid analogs may refer to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but functions in a manner like a naturally occurring amino acid. The synthetically modified forms include, but are not limited to, amino acids having side chains shortened or lengthened by up to two carbon atoms, amino acids comprising optionally substituted aryl groups, and amino acids comprised halogenated groups, preferably halogenated alkyl and aryl groups and also N substituted amino acids e.g., N-methyl-alanine. An amino acid or peptide can be attached to a linker/spacer or a cytotoxic agent through the terminal amine or terminal carboxylic acid of the amino acid or peptide. The amino acid can also be attached to a linker/spacer or a cytotoxic agent through a side chain reactive group, such as but not restricted to the thiol group of cysteine, the epsilon amine of lysine or the side chain hydroxyls of serine or threonine.

Amino acids and peptides may be protected by blocking groups. A blocking group is an atom or a chemical moiety that protects the N-terminus of an amino acid or a peptide from undesired reactions and can be used during the synthesis of a drug-ligand conjugate. It should remain attached to the N-terminus throughout the synthesis and may be removed after completion of synthesis of the drug conjugate by chemical or other conditions that selectively achieve its removal. The blocking groups suitable for N-terminus protection are well known in the art of peptide chemistry. Exemplary blocking groups include, but are not limited to, methyl esters, tert-butyl esters, 9-fluorenylmethyl carbamate (Fmoc) and carbobenzoxy (Cbz).

### Platelet Like Cells and Extracellular Vesicles

In some embodiments, the present disclosure provides non-naturally existing, novel, anucleated platelet like cells (PLCs) or derivatives thereof that otherwise do not exist in nature. In some embodiments, the present disclosure provides non-naturally existing extracellular vesicles or derivatives thereof that otherwise do not exist in nature. The PLCs and EVs are generally produced as admixtures and one can be isolated from one another, at least based on their size difference.

The PLCs and the EVs or derivatives thereof of the present disclosure are non-tumorigenic, essentially non-immunogenic and migratory cells whereby the PLCs and/or the EVs or derivatives thereof utilize their systemic (i.e., can be distributed into interstitial and intracellular fluids) or/and rolling, adhesion, and aggregate formation capabilities to travel (roll) from a first location, where the PLCs or the EVs or their derivatives are administered, to a second location i.e., a diseased location, where the PLCs and/or EVs or their derivatives adhere and aggregate at the diseased target to mitigate or eliminate the disease.

In some embodiments, the PLCs of the present disclosure are structurally characterized as:

CD63^{>average60%}, as compared to the reference resting bone marrow derived platelet cells with the structure CD63^{<average 2%}.

CD63^{>average55%} as compared to the reference resting bone marrow derived platelet cells with the structure CD63^{<average 2%}.

CD63^{>average50%}, as compared to the reference resting bone marrow derived platelet cells with the structure CD63^{<average 2%}.

CD63^{>average45%}, as compared to the reference resting bone marrow derived platelet cells with the structure CD63^{<average 2%}.

CD63^{>average40%}, as compared to the reference resting bone marrow derived platelet cells with the structure CD63^{<average 2%}.

CD63^{>average35%} as compared to the reference resting bone marrow derived platelet cells with the structure CD63^{<average 2%}.

CD63^{>average30%}, as compared to the reference resting bone marrow derived platelet cells with the structure CD63^{<average 2%}.

CD63^{>average25%}, as compared to the reference resting bone marrow derived platelet cells with the structure CD63^{<average 2%}.

CD63^{>average20%}, as compared to the reference resting bone marrow derived platelet cells with the structure CD63^{<average 2%}.

CD63^{>average 15%} as compared to the reference resting bone marrow derived platelet cells with the structure CD63^{<average 2%}.

CD63^{>average10%}, as compared to the reference resting bone marrow derived platelet cells with the structure CD63^{<average 2%}.

CD63^{>average5%} as compared to the reference resting bone marrow derived platelet cells with the structure CD63^{<average 2%}.

CD63^{>average2%}, as compared to the reference resting bone marrow derived platelet cells with the structure CD63^{<average 2%.}

In some embodiments, the PLCs of the present disclosure are structurally characterized as:

CD61^{<average98%} as compared to the reference resting bone marrow derived platelet cells with the structure CD61^{>average 98%}).

CD61^{<average88%} as compared to the reference resting bone marrow derived platelet cells with the structure CD61^{>average 98%}).

CD61^{<average78%} as compared to the reference resting bone marrow derived platelet cells with the structure CD61^{>average 98%}).

CD61^{<average68%}, as compared to the reference resting bone marrow derived platelet cells with the structure CD61^{>average 98%}).

CD61^{<average58%} as compared to the reference resting bone marrow derived platelet cells with the structure CD61^{>average 98%}).

CD61^{<average48%}, as compared to the reference resting bone marrow derived platelet cells with the structure CD61^{>average 98%}).

CD61^{<average38%} as compared to the reference resting bone marrow derived platelet cells with the structure CD61^{>average 98%}).

CD61^{<average28%}, as compared to the reference resting bone marrow derived platelet cells with the structure CD61^{>average 98%}).

In some embodiments, the present disclosure comprises a variant of a resting reference bone marrow derived platelet cell comprising the structure CD63^{>average2%}, CD61^{<average96%}) as compared to the reference resting bone marrow derived platelet cells with the structure CD63^{<average 2%}, CD61^{>average 96%}.

In some embodiments, the PLCs of the present disclosure are structurally characterized as:

CD63^{>average60%} CD61^{<average 96%} as compared to the reference resting bone marrow derived platelet cells with the structure CD63^{<average 2%} CD61^{>average 96%}.

CD63^{>average55%} CD61^{<average 96%} as compared to the reference resting bone marrow derived platelet cells with the structure CD63^{<average 2%} CD61^{>average 96%}.

CD63^{>average 50%} CD61^{<average 96%} as compared to the reference resting bone marrow derived platelet cells with the structure CD63^{<average 2%} CD61^{>average 96%}.

CD63^{>average45%} CD61^{<average 96%} as compared to the reference resting bone marrow derived platelet cells with the structure CD63^{<average 2%} CD61^{>average 96%}.

CD63^{>average40%} CD61^{<average 96%} as compared to the reference resting bone marrow derived platelet cells with the structure CD63^{<average 2%} CD61^{>average 96%}.

CD63^{>average35%} CD61^{<average 96%} as compared to the reference resting bone marrow derived platelet cells with the structure CD63^{<average 2%} CD61^{>average 96%}.

CD63^{>average 30%} CD61^{<average 96%} as compared to the reference resting bone marrow derived platelet cells with the structure CD63^{<average 2%} CD61^{>average 96%}.

CD63^{>average25%} CD61^{<average 96%} as compared to the reference resting bone marrow derived platelet cells with the structure CD63^{<average 2%} CD61^{>average 96%}.

CD63^{>average20%} CD61^{<average 96%} as compared to the reference resting bone marrow derived platelet cells with the structure CD63^{<average 2%} CD61^{>average 96%}.

CD63^{>average15%} CD61^{<average 96%} as compared to the reference resting bone marrow derived platelet cells with the structure CD63^{<average 2%} CD61^{>average 96%}.

CD63^{>average10%} CD61^{<average 96%} as compared to the reference resting bone marrow derived platelet cells with the structure CD63^{<average 2%} CD61^{>average 96%}.

CD63^{>average5%} CD61^{<average 96%} as compared to the reference resting bone marrow derived platelet cells with the structure CD63^{<average 2%} CD61^{>average 96%}.

CD63^{>average2%} CD61^{<average 96%} as compared to the reference resting bone marrow derived platelet cells with the structure CD63^{<average 2%} CD61^{>average 96%}.

In some embodiments, the present disclosure comprises a variant of a resting reference bone marrow derived platelet cell comprising the structure CD63^{>average2%}, CD42b^{<average 99%} as compared to the reference resting bone marrow derived platelet cells with the structure CD63^{<average 2%} CD42b^{>average 99%}.

In some embodiments, the PLCs of the present disclosure are structurally characterized as:

CD63^{>average60%}, CD42b^{<average 38%} as compared to the reference resting bone marrow derived platelet cells with the structure CD63^{<average 2%} CD42b^{>average 99%}.

CD63^{>average55%}, CD42b^{<average 38%} as compared to the reference resting bone marrow derived platelet cells with the structure CD63^{<average 2%} CD42b^{>average 99%}.

CD63^{>average50%}, CD42b^{<average 38%} as compared to the reference resting bone marrow derived platelet cells with the structure CD63^{<average 2%} CD42b^{>average 99%}.

CD63^{>average45%}, CD42b^{<average 38%} as compared to the reference resting bone marrow derived platelet cells with the structure CD63^{<average 2%} CD42b^{>average 99%}.

CD63^{>average40%}, CD42b^{<average 38%} as compared to the reference resting bone marrow derived platelet cells with the structure CD63^{<average 2%} CD42b^{>average 99%}.

CD63^{>average35%}, CD42b^{<average 38%} as compared to the reference resting bone marrow derived platelet cells with the structure CD63^{<average 2%} CD42b^{>average 99%}.

CD63^{>average30%}, CD42b^{<average 38%} as compared to the reference resting bone marrow derived platelet cells with the structure CD63^{<average 2%} CD42b^{>average 99%}.

CD63^{>average25%}, CD42b^{<average 38%} as compared to the reference resting bone marrow derived platelet cells with the structure CD63^{<average 2%} CD42b^{>average 99%}.

CD63^{>average20%}, CD42b^{<average 38%} as compared to the reference resting bone marrow derived platelet cells with the structure CD63^{<average 2%} CD42b^{>average 99%}.

CD63^{>average15%}, CD42b^{<average 38%} as compared to the reference resting bone marrow derived platelet cells with the structure CD63^{<average 2%} CD42b^{>average 99%}.

CD63^{>average 10%}, CD42b^{<average 38%} as compared to the reference resting bone marrow derived platelet cells with the structure CD63^{<average 2%} CD42b^{>average 99%}.

CD63^{>average5%}, CD42b^{<average 38%} as compared to the reference resting bone marrow derived platelet cells with the structure CD63^{<average 2%} CD42b^{>average 99%}.

CD63^{>average2%}, CD42b^{<average 38%} as compared to the reference resting bone marrow derived platelet cells with the structure CD63^{<average 2%} CD42b^{>average 99%}.

In some embodiments, the present disclosure comprises a variant of a resting reference bone marrow derived platelet cell comprising the structure CD63^{>average2%}CD36^{<average 92%} as compared to the reference resting bone marrow derived platelet cells with the structure CD63^{<average 2%}, CD36^{>average 92%}.

In some embodiments, the PLCs of the present disclosure are structurally characterized as:

CD63^{>average60%} CD36^{<average 5%} as compared to the reference resting bone marrow derived platelet cells with the structure CD63^{<average 2%}, CD36^{>average 92%}.

CD63^{>average55%}, CD36^{<average 5%}, as compared to the reference resting bone marrow derived platelet cells with the structure CD63^{<average 2%}, CD36^{>average 92%}.

CD63^{>average50%}, CD36^{<average 5%} as compared to the reference resting bone marrow derived platelet cells with the structure CD63^{<average 2%}, CD36^{>average 92%}.

CD63^{>average45%}, CD36^{<average 5%} as compared to the reference resting bone marrow derived platelet cells with the structure CD63^{<average 2%}, CD36^{>average 92%}.

CD63^{>average40%}, CD36^{<average 5%} as compared to the reference resting bone marrow derived platelet cells with the structure CD63^{<average 2%} CD36^{>average 92%}.

CD63^{>average35%} CD36^{<average 5%} as compared to the reference resting bone marrow derived platelet cells with the structure CD63^{<average 2%} CD36^{>average 92%}.

CD63^{>average30%} CD36^{<average 5%} as compared to the reference resting bone marrow derived platelet cells with the structure CD63^{<average 2%} CD36^{>average 92%}.

CD63^{>average25%} CD36^{<average 5%} as compared to the reference resting bone marrow derived platelet cells with the structure CD63^{<average 2%} CD36^{>average 92%}.

CD63^{>average20%} CD36^{<average 5%} as compared to the reference resting bone marrow derived platelet cells with the structure CD63^{<average 2%} CD36^{>average 92%}.

CD63^{>average15%} CD36^{<average 5%} as compared to the reference resting bone marrow derived platelet cells with the structure CD63^{<average 2%} CD36^{>average 92%}.

CD63^{>average10%} CD36^{<average 5%} as compared to the reference resting bone marrow derived platelet cells with the structure CD63^{<average 2%} CD36^{>average 92%}.

CD63^{>average5%} CD36^{<average 5%} as compared to the reference resting bone marrow derived platelet cells with the structure CD63^{<average 2%} CD36^{>average 92%}.

CD63^{>average2%} CD36^{<average 5%} as compared to the reference resting bone marrow derived platelet cells with the structure CD63^{<average 2%} CD36^{>average 92%}.

In some embodiments, the PLCs of the present disclosure are structurally characterized as:

CD63^{>average60%} GPVI^{<average 5%} as compared to the reference resting bone marrow derived platelet cells with the structure CD63^{<average 2%} GPVI^{<average 92%}.

CD63^{>average55%} GPVI^{<average 5%} as compared to the reference resting bone marrow derived platelet cells with the structure CD63^{<average 2%} GPVI^{<average 92%}.

CD63^{>average50%} GPVI^{<average 5%} as compared to the reference resting bone marrow derived platelet cells with the structure CD63^{<average 2%} GPVI^{<average 92%}.

CD63^{>average45%} GPVI^{<average 5%} as compared to the reference resting bone marrow derived platelet cells with the structure CD63^{<average 2%} GPVI^{<average 92%}.

CD63^{>average40%} GPVI^{<average 5%} as compared to the reference resting bone marrow derived platelet cells with the structure CD63^{<average 2%} GPVI^{<average 92%}.

CD63^{>average35%} GPVI^{<average 5%} as compared to the reference resting bone marrow derived platelet cells with the structure CD63^{<average 2%} GPVI^{<average 92%}.

CD63^{>average30%} GPVI^{<average 5%} as compared to the reference resting bone marrow derived platelet cells with the structure CD63^{<average 2%} GPVI^{<average 92%}.

CD63^{>average25%} GPVI^{<average 5%} as compared to the reference resting bone marrow derived platelet cells with the structure CD63^{<average 2%} GPVI^{<average 92%}.

CD63^{>average20%} GPVI^{<average 5%} as compared to the reference resting bone marrow derived platelet cells with the structure CD63^{<average 2%} GPVI^{<average 92%}.

CD63^{>average15%} GPVI^{<average 5%} as compared to the reference resting bone marrow derived platelet cells with the structure CD63^{<average 2%} GPVI^{<average 92%}.

CD63^{>average10%} GPVI^{<average 5%} as compared to the reference resting bone marrow derived platelet cells with the structure CD63^{<average 2%} GPVI^{<average 92%}.

CD63^{>average5%} GPVI^{<average 5%} as compared to the reference resting bone marrow derived platelet cells with the structure CD63^{<average 2%} GPVI^{<average 92%}.

CD63^{>average2%} GPVI^{<average 5%} as compared to the reference resting bone marrow derived platelet cells with the structure CD63^{<average 2%} GPVI^{<average 92%}.

In some embodiments, the PLCs of the present disclosure are structurally characterized as:

CD63^{>average60%} CD41a^{>average 77%} as compared to the reference resting bone marrow derived platelet cells with the structure CD63^{<average 2%} CD41a^{>average 99%}.

CD63^{>average55%} CD41a^{>average 77%} as compared to the reference resting bone marrow derived platelet cells with the structure CD63^{<average 2%} CD41a^{>average 99%}.

CD63^{>average50%} CD41a^{>average 77%} as compared to the reference resting bone marrow derived platelet cells with the structure CD63^{<average 2%} CD41a^{>average 99%}.

CD63^{>average45%} CD41a^{>average 77%} as compared to the reference resting bone marrow derived platelet cells with the structure CD63^{<average 2%} CD41a^{>average 99%}.

CD63^{>average40%} CD41a^{>average 77%} as compared to the reference resting bone marrow derived platelet cells with the structure CD63^{<average 2%} CD41a^{>average 99%}.

CD63^{>average35%} CD41a^{>average 77%} as compared to the reference resting bone marrow derived platelet cells with the structure CD63^{<average 2%} CD41a^{>average 99%}.

CD63^{>average30%} CD41a^{>average 77%} as compared to the reference resting bone marrow derived platelet cells with the structure CD63^{<average 2%} CD41a^{>average 99%}.

CD63^{>average25%} CD41a^{>average 77%} as compared to the reference resting bone marrow derived platelet cells with the structure CD63^{<average 2%} CD41a^{>average 99%}.

CD63^{>average20%} CD41a^{<average 77%} as compared to the reference resting bone marrow derived platelet cells with the structure CD63^{<average 2%} CD41a^{>average 99%}.

CD63^{>average 15%} CD41a^{>average 77%} as compared to the reference resting bone marrow derived platelet cells with the structure CD63^{<average 2%} CD41a^{>average 99%}.

CD63^{>average 10%} CD41a^{>average 77%} as compared to the reference resting bone marrow derived platelet cells with the structure CD63^{<average 2%} CD41a^{>average 99%}.

CD63^{>average5%} CD41a^{<average 77%} as compared to the reference resting bone marrow derived platelet cells with the structure CD63^{<average 2%} CD41a^{>average 99%}.

CD63^{>average2%} CD41a^{<average 77%} as compared to the reference resting bone marrow derived platelet cells with the structure CD63^{<average 2%} CD41a^{>average 99%}.

In some embodiments, the PLCs of the present disclosure are structurally characterized as:

CD63^{>average 2%}-TLT-1^{<average 13%};

CD63^{>average 2%}-TLT-1^{<average 13%}-CD42b^{<average 38%};

CD63^{>average 2%}-TLT-1^{<average 13%}-CD42b^{<average 38%}-CD36^{<average 5%}.

CD63^{>average 2%}-TLT-1^{<average 13%}-CD42b^{<average 38%}-CD36^{<average 5%}-GPVI^{<average 5%};

CD63^{>average 2%}-TLT-1^{<average 13%}-CD42b^{<average 38%}-CD36^{<average 5%}-GPVI^{<average 5%}

-CD61^{<average 66%};

CD63^{>average 2%}-TLT-1^{<average 13%}-CD42b^{<average 38%}-CD36^{<average 5%}-GPVI^{<average 5%}-CD61^{<average66%}-CD42a^{<average 75%}.

CD63^{>average 2%}-TLT-1^{<average 13%}-CD42b^{<average 38%}-CD36^{<average 5%}-GPVI^{<average 5%}-CD61^{<average66%}-CD42a^{<average 75%}-, CD41a^{<average77%}; or

CD63^{>average2%}-CD42b^{<average 38%}-CD36^{<average 5%}-GPVI^{<average 5%} or variations thereof, which simply do not exist in bone marrow derived platelet cells, resting or otherwise.

In some embodiments, at the very least, the PLCs of the present disclosure, in their resting stage, have higher concentrations of CD63 receptors, for example, at least by 2% greater as compared to CD63 concentration in naturally existing bone marrow derived platelets.

In some embodiments, the non-naturally occurring PLCs in their resting stage have CD63 concentrations that is 90%, 80%, 70%, 60% 50%, 40%, 30%, 20%, 15%, 10%, 5% or 1% greater than the CD63 concentration as compared to the resting bone marrow derived platelets. In some embodiments, the non-naturally occurring PLCs in their resting stage have a CD63 concentration that is between 100% to 90%, between 90% to 80%, between 80% to 70% between 70% to 60%, between 60% to 50%, between 50% to 40%, between 40 to 30%, between 30% to 20%, between 20% to 15%, between 15% to 10%, between 10% to 5% or 5% to 1% greater than the CD63 concentration as compared to the resting bone marrow derived platelets.

In some embodiments, the non-naturally occurring PLCs in the presence of a CD63 agonist have a CD63 concentration that is 90%, 80%, 70%, 60% 50%, 40%, 30%, 20%, 15%, 10%, 5% or 1% less than the CD63 concentration as compared to CD63-agonist activated bone marrow derived platelets. In some embodiments, the non-naturally occurring PLCs in presence of CD63 agonist have a CD63 concentration that is between 100% to 90%, between 90% to 80%, between 80% to 70% between 70% to 60%, between 60% to 50%, between 50% to 40%, between 40 to 30%, between 30% to 20%, between 20% to 15%, between 15% to 10%, between 10% to 5% or 5% to 1% less than the CD63 concentration as compared to CD63 agonist-activated naturally existing bone marrow derived platelets.

In some embodiments, the non-naturally occurring PLCs in their resting stage have CD61 concentrations that is 90%, 80%, 70%, 60% 50%, 40%, 30%, 20%, 15%, 10%, 5% or 1% less than the CD61 concentration as compared to the resting bone marrow derived platelets. In some embodiments, the non-naturally occurring PLCs in their resting stage have a CD61 concentration that is between 100% to 90%, between 90% to 80%, between 80% to 70% between 70% to 60%, between 60% to 50%, between 50% to 40%, between 40 to 30%, between 30% to 20%, between 20% to 15%, between 15% to 10%, between 10% to 5% or 5% to 1% less than the CD61 concentration as compared to the resting bone marrow derived platelets.

In some embodiments, concurrent with CD63 expression, agonist activated PLCs have 90%, 80%, 70%, 60% 50%, 40%, 30%, 20%, 15%, 10%, 5% or 1% less concentration of one or more of TLT1, PAC1 CD62p receptors as compared to agonist activated TLT1, PAC1, or CD62p receptors in naturally existing bone marrow derived platelets. In some embodiments, agonist activated PLCs have one or more of TLT1, PAC1 or CD62p have between 100% to 90%, between 90% to 80%, between 80% to 70% between 70% to 60%, between 60% to 50%, between 50% to 40%, between 40 to 30%, between 30% to 20%, between 20% to 15%, between 15% to 10%, between 10% to 5% or 5% to 1% less concentration of TLT1, PAC1 CD62p as compared to agonist activated TLT1, PAC1 or CD62p receptors in the naturally existing bone marrow derived platelets.

In some embodiments, concurrent with CD63 concentration, PLCs concentration of one or more of CD42b and CD36 receptors is 90%, 80%, 70%, 60% 50%, 40%, 30%, 20%, 15%, 10%, 5% or 1% or less as compared to the concentration of CD42b and CD36 in naturally existing bone marrow derived platelets. In some embodiments, concurrent with CD63 concentration, PLCs concentration of one or more of CD42b and CD36 receptors is between 100% to 90%, between 90% to 80%, between 80% to 70% between 70% to 60%, between 60% to 50%, between 50% to 40%, between 40 to 30%, between 30% to 20%, between 20% to 15%, between 15% to 10%, between 10% to 5% or 5% to 1% less as compared to CD42b and/or CD36 concentration in the naturally existing bone marrow derived platelets.

In some embodiments, concurrent with CD63 concentration, concentration of one or more of GPVI, calcein, PAC1 CD42d or CD42bad is 90%, 80%, 70%, 60% 50%, 40%, 30%, 20%, 15%, 10%, 5% or 1% less as compared to the concentration of one or more of GPVI, calcein, PAC1 CD42d or CD42bad in the naturally existing bone marrow derived platelets. In some embodiments, concurrent with CD63 concentration, concentration of one or more of GPVI, calcein, PAC1 CD42d or CD42bad is between 100% to 90%, between 90% to 80%, between 80% to 70% between 70% to 60%, between 60% to 50%, between 50% to 40%, between 40 to 30%, between 30% to 20%, between 20% to 15%, between 15% to 10%, between 10% to 5% or 5% to 1% less as compared to the concentration of one or more of GPVI, calcein, PAC1 CD42d or CD42bad in the naturally existing bone marrow derived platelets.

In some embodiments, the non-naturally occurring PLCs in their resting stage have lactadherin concentration that is 90%, 80%, 70%, 60% 50%, 40%, 30%, 20%, 15%, 10%, 5% or 1% greater as compared to the concentration of lactadherin in resting naturally existing bone marrow derived platelets. In some embodiments, the non-naturally occurring PLCs in their resting stage have a lactadherin concentration that is between 100% to 90%, between 90% to 80%, between 80% to 70% between 70% to 60%, between 60% to 50%, between 50% to 40%, between 40 to 30%, between 30% to 20%, between 20% to 15%, between 15% to 10%, between 10% to 5% or 5% to 1% greater as compared to the concentration of lactadherin in resting naturally existing bone marrow derived platelets.

In some embodiments, concurrent with the lack of or depleted CD63 agonist dependent activation, the same PLCs are also devoid of or show depleted responses to agonists directed to one or more of TLT1, PAC1, or CD62p upon activation by the corresponding TLT1, PAC1, or CD62p agonists, unlike in the naturally existing bone marrow derived platelets where the TLT1, PAC1, or CD62p are robustly activated by their corresponding agonists.

In some embodiments, the same PLCs, concurrent with higher concentration of CD63 in their resting stage, are essentially devoid of or are depleted in CD42b and/or CD36 receptor concentration, unlike in the naturally existing bone marrow derived platelets where the CD42b and CD36 concentrations are robust in the resting bone-marrow derived platelets.

In some embodiments, the same PLCs, concurrent with the higher concentration of CD63 in their resting stage, are essentially devoid of or are depleted in glycoprotein VI and CD42a receptor concentration, unlike in the naturally existing bone marrow derived platelets where the glycoprotein VI and CD42a concentrations are robust in the resting bone-marrow derived platelets.

In some embodiments, the PLCs, concurrent with the higher concentration of CD63 in their resting stage, also have higher concentration of lactadherin in resting stage, unlike in the naturally existing bone marrow derived platelets where the lactadherin concentration is depleted in the resting bone-marrow derived platelets.

In some embodiments, the same PLCs, concurrent with the higher concentration of CD63 in their resting stage, are essentially devoid of or are depleted in PAC-1 concentration, unlike in the naturally existing bone marrow derived platelets where the PAC-1 concentration is robust in the resting bone-marrow derived platelets. In some embodiments, PAC-1, in the PLCs, are devoid of agonistic activation by PAC-1 agonist, unlike in the naturally existing resting bone marrow derived platelets where PAC-1 is readily activated by PAC-1 agonists.

In some embodiments, the PLCs, concurrent with the higher concentration of CD63 in their resting stage, are essentially devoid of or are depleted in one or more of CD42d or CD42bad receptor or calcein marker concentration, unlike in the naturally existing bone marrow derived platelets where each of CD42d or CD42bad or calcein concentrations are relatively higher in the resting bone-marrow derived platelets.

In some embodiments, the structural makeup of the PLCs are comparable to the structural make-up of naturally existing bone marrow derived platelets, either in resting or in their activated stages. For example, the PLCs and the donor platelets may have m % CD36, n % CD42a, o % CD42a-b-d, p % CD61, q % CD62p, or x % CD63 receptors, where the m %, n %, o %, p %, q % x % are the same (i.e., have equal values) between the PLCs and the and bone marrow derived platelets. In other words, structurally PLCs may be identical to donor platelets, yet manifest the advantages of the PLC variants disclosed herein in this application. For example, structurally PLCs and donor platelets may be identical in term of their receptor or ligand, but differ in their size or functionalities.

In some embodiments, the PLCs of the present disclosure more robustly catalyze the activation of the blood clotting cascade as measured by the release of thrombin in plasma. The PLCs, as compared to their natural counterpart (i.e., bone marrow derived platelets) may catalyze the release of thrombin in an amount that is 1-5-fold, 5-10-fold, 10-15-fold, 15-20-fold, 20-25-fold, 25-30-fold, 35-40-fold, 40-45-fold, 45-50-fold, 50-55-fold, 55-60-fold, 60-65-fold, 65-70-fold, 70-75-fold, 75-80-fold, 80-85-fold, 85-90-fold, 90-95-fold, 95-100-fold, greater than the amount of thrombin that is released by the naturally existing bone marrow derived platelets.

In some embodiments, the PLCs may catalyze the activation of the blood clotting cascade much more rapidly as compared to their naturally existing bone marrow derived platelets. For example, PLCs, upon stimulus from an agent such as Factor VIII, release thrombin as early as between 0-5 minutes, 5-10 minutes, 10-15 minutes, 15-20 minutes 20-25 minutes or in recurring administrations of PLCs between 30-60 minutes or later, such as between 1-2 hours, 2-4 hours and 6-8 hours, 8-10 hours, 10-12 hours, 12-14 hours post stimulation, in contrast to bone marrow derived platelets. PLCs may be transfused into a patient in need of activation of the blood clotting cascade once, twice or multiple times on an hourly, daily or weekly basis per the need of the patient.

The PLCs of the present disclosure are advantageous over the naturally existing bone marrow derived platelets because PLCs, made ex-vivo/in-vitro, are readily available in greater or unlimited supply as compared to donor platelets. Therefore, in some embodiments of the present disclosure, the PLCs or derivatives thereof of the present disclosure can be used to rapidly replenish platelets in patients in need of platelet transfusion. Moreover, the PLCs, made in large volumes in PLC bioreactors, makes it easy to store and transfer PLCs or derivatives thereof to remote locations. Also, the PLCs eliminate the need of platelet donors or human volunteers to donate naturally existing bone marrow derived platelets. Furthermore, based at least on the characteristics of the PLCs to rapidly catalyze thrombin induced blood clotting cascade, PLCs or derivatives thereof can, in some embodiments, be used as a thrombin activated responder to rapidly stimulate blood clotting cascades in bleeding injuries in war zones or in bleeding injuries from natural disasters, where PLCs provide life-saving advantage of stopping bleeding early on by enhancing thrombin-induced hemostasis, which otherwise may be limited by the lack in supply of donor platelets or by the loss of platelets during bleedings. Likewise, EVs (discussed below), made ex-vivo like the PLCs, are readily available in greater or unlimited supply and being rich in thrombin or other factors (e.g., growth factors, enzymes etc.), may supplement the PLCs by providing life-saving advantage of stopping bleeding early on by enhancing thrombin-induced hemostasis.

PLCs or derivatives thereof of the present disclosure may also be used to supplement the functioning of naturally existing bone marrow derived platelets. For example, the PLCs catalyze thrombin release or generate thrombin early on, as compared to naturally existing bone marrow derived platelets, thus the PLCs may advantageously be added to the naturally existing bone marrow derived platelets whereby the thrombin activation by the PLCs stimulate the activation of the naturally existing bone marrow derived platelets to enhance hemostasis is desired, for example to stop bleeding. For example, PLCs' thrombin-induced activation produces a highly efficient catalytic surface for the generation of thrombin on the endogenous or the naturally existing bone marrow derived platelets, thereby enhancing hemostasis. Thrombin is key mediator of platelet activation, release reaction and aggregation. Thrombin's action on naturally existing bone marrow derived platelets leads to fibrin clot formation, hence wound healing. In this case, PLCs' thrombin is expected to activate the endogenous or the naturally existing bone marrow derived platelets leading the PLCs as well as the endogenous or naturally existing bone marrow derived platelets to contribute to blood coagulation process during the wound healing. EVs or derivatives thereof may complement the PLCs in this role.

### Extracellular Vesicles (EV)

In some embodiments, the present disclosure comprises microvesicles and exosomes (collectively referred to as extracellular vesicles (EV)) or derivatives thereof, which are produced admixtures of the PLCs. Given that EVs or derivatives thereof carry receptors, bioactive lipids, nucleic acids, such as mRNA and microRNA (miRNA) or siRNA, proteins, they are able to deliver important payloads to recipient cells (e.g., tumor cells).

EVs or derivatives thereof of the present disclosure can be isolated and purified, essentially separating them from an admixture comprising the PLCs of the disclosure. Isolated or purified extracellular vesicles (EV) or derivatives thereof, because of their ability to extensively travel throughout the body, can exert remarkable therapeutic effects when administered to a patient on their own. Advantageously, EVs or derivatives thereof can be internalized by recipient cells following receptor-ligand interactions and the varied assortment of bioactive molecules, derived from the cell of origin, such as proteins, bioactive lipids, and nucleic acids, can be transferred along with the proteins expressed on the EV surface.

Thus, in some embodiments, the disclosure provides a composition that includes a population of extracellular vesicles derived from induced pluripotent stem cell (iPSC), where the extracellular vesicles exhibit increased thrombin generation relative to a population of extracellular vesicles derived from donor derived platelets, and where the populations of extracellular vesicles are derived from about the same number of iPSC derived platelets and donor derived platelets.

In some embodiments, the thrombogenic activity of the extracellular vesicles (EV) or derivatives thereof derived from induced pluripotent stem iPSC derived platelets is greater than the thrombogenic activity present in a microparticle derived from a donor derived platelet or megakaryocyte. In some embodiments, the thrombogenic activity present in the microparticle results in a maximum concentration of about 400 nM thrombin.

In some embodiments, EVs or derivatives thereof may directly activate a recipient cell (e.g., donor platelets) by acting as signaling complexes. For example, EVs or derivatives thereof may bind to platelets by means of the P-selectin glycoprotein ligand-1 expressed on their surface and EVs or derivatives thereof from neutrophils expressing Mac-1 may induce donor platelet activation in a patient in need thereof.

Compositions and methods comprising the extracellular vesicles (EV) or derivatives thereof of the present disclosure can be used in several therapies, such as delivery of genes, proteins or peptides, nucleic acids for the use in cellular or gene therapies, for example using vectors, e.g., adenovirus, lentivirus, to obtain novel microvesicles or exosomal gene (e.g., for gene therapy), peptide (for growth factors) or nucleic acid (e.g., siRNA or microRNA) delivery vehicles. Packaging within extracellular vesicles (EV) provides several advantages such as shielding the molecules from adverse cellular event that may neutralize the naked gene. Engineered extracellular vesicles (EV) could be used to carry drugs to specific sites of tissue damage, including but not limited to cancer, Alzheimer and other disorders discussed elsewhere herein.

The extracellular vesicles (EV) or derivatives thereof of the present disclosure are isolated as described in the experimental section of the description. The isolated extracellular vesicles (EV) derivatives thereof may then be stored until use by freezing at very low temperature, e.g., at −80° C. in presence of cryopreserving agents, such dimethylsulphoxide (DMSO) and glycerol used at optimal concentrations.

In some embodiments, an average diameter of extracellular vesicles (EV) derived from a population of iPSC derived platelets is less than 50% the diameter of the extracellular vesicles (EV) derived from a population of donor derived platelets having about the same number of platelets as the population of iPSC derived platelets. In some embodiments, the megakaryocyte or platelet is genetically modified to comprise a nucleic acid molecule encoding a therapeutic agent.

Extracellular vesicles (EV) are subcellular sized particles consisting of a membrane lipid bilayer and cellular content. Extracellular vesicles (EV) isolated or purified from an admixture comprising PLCs may exert both anti-inflammatory or pro-inflammatory functions and have potential as vehicles for drug delivery. In some embodiments, the instant extracellular vesicles (EV) are able to produce thrombin.

In some embodiments, the diameter of the instant extracellular vesicles (EV) is 0.1 and 4 μm. In some embodiments, the diameter of the instant extracellular vesicles (EV) is 0.1 and 3 μm. In some embodiments, the diameter of the instant extracellular vesicles (EV) is 0.1 and 2.5 μm. In some embodiments, the diameter of the instant extracellular vesicles (EV) is 0.1 and 2 μm. In some embodiments, the diameter of the instant extracellular vesicles (EV) is 0.1 and 1.5 μm. In some embodiments, the diameter of the instant extracellular vesicles (EV) is 0.1 and 1.0 μm. In some embodiments, the diameter of the instant extracellular vesicles (EV) is 0.1 and 0.9 μm. In some embodiments, the diameter of the instant extracellular vesicles (EV) is 0.1 and 0.8 μm. In some embodiments, the diameter of the instant extracellular vesicles (EV) is 0.1 and 0.7 μm. In some embodiments, the diameter of the instant extracellular vesicles (EV) is 0.1 and 0.6 μm. In some embodiments, the diameter of the instant extracellular vesicles (EV) is 0.1 and 0.5 μm. In some embodiments, the diameter of the instant extracellular vesicles (EV) is 0.1 and 0.4 μm. In some embodiments, the diameter of the instant extracellular vesicles (EV) is 0.1 and 0.3 μm. In some embodiments, the diameter of the instant extracellular vesicles (EV) is 0.1 and 0.2 μm. In some embodiments, the diameter of the instant extracellular vesicles (EV) is 0.2 and 1 μm. In some embodiments, the diameter of the instant extracellular vesicles (EV) is 0.3 and 1 μm. In some embodiments, the diameter of the instant extracellular vesicles (EV) is 0.4 and 1 μm. In some embodiments, the diameter of the instant extracellular vesicles (EV) is 0.5 and 1 μm. In some embodiments, the diameter of the instant extracellular vesicles (EV) is 0.6 and 1 μm. In some embodiments, the diameter of the instant extracellular vesicles (EV) is 0.7 and 1 μm. In some embodiments, the diameter of the instant extracellular vesicles (EV) is 0.8 and 1 μm. In some embodiments, the diameter of the instant extracellular vesicles (EV) is 0.9 and 1 μm. In some embodiments, the diameter of the instant extracellular vesicles (EV) is 0.2 and 2 μm. In some embodiments, the diameter of the instant extracellular vesicles (EV) is 0.3 and 2 μm. In some embodiments, the diameter of the instant extracellular vesicles (EV) is 0.4 and 2 μm. In some embodiments, the diameter of the instant extracellular vesicles (EV) is 0.5 and 2 μm. In some embodiments, the diameter of the instant extracellular vesicles (EV) is 0.6 and 2 μm. In some embodiments, the diameter of the instant extracellular vesicles (EV) is 0.7 and 2 μm. In some embodiments, the diameter of the instant extracellular vesicles (EV) is 0.8 and 2 μm. In some embodiments, the diameter of the instant extracellular vesicles (EV) is 0.9 and 2 μm. In some embodiments, the diameter of the instant extracellular vesicles (EV) is 1.0 and 2 μm. In some embodiments, the diameter of the instant extracellular vesicles (EV) is 1.5 and 2 μm. In some embodiments, the diameter of the instant extracellular vesicles (EV) is 1.5 and 2.5 μm. In some embodiments, the diameter of the instant extracellular vesicles (EV) is 2.0 and 2.5 μm.

In some embodiments, thrombogenic compositions of extracellular vesicles (EV) are provided such that the composition has a peak size of less than approximately 2 μm. In some embodiments, the thrombogenic extracellular vesicles (EV) range in size between 40 nm and 100 nm in diameter. In some embodiments, the thrombogenic extracellular vesicles (EV) form greater than 50% of the composition. In some embodiments, the thrombogenic extracellular vesicles (EV) form greater than 60%, 70%, 80%, 90% or 100% of the composition.

Extracellular vesicles (EV) derivatives thereof may be conjugated to one or more cytotoxic agents by mechanisms disclosed in the foregoing. One or more cytotoxic agents may be imbibed into the extracellular vesicles (EV) derivatives thereof by mechanisms also disclosed in the foregoing. Cytotoxic agents are also disclosed in the foregoing. Diseases and disorders that can be cured or mitigated by the use of EVs derivatives thereof alone or in combination with the PLCs or derivatives thereof of the present disclosure are also disclosed below.

In some embodiments, EVs, whether modified or not (e.g., bioengineered or conjugated) may be developed for therapeutic use independent of the PLCs or derivatives therefrom. For example, a patient in need of a treatment predominantly involving microvesicles or derivatives thereof will be administered microvesicle-based treatment or exosome-based treatment or a combination of both. For example, MVs or exosomes incorporated with exogenous siRNAs can be used for efficient silencing of a target MAPK gene in monocytes and lymphocytes or deliver VEGF-siRNA across the blood-brain barrier or siRNAs targeting Huntingtin disease, or siRNA into the liver, among others. advantageously, MVs could be used as more efficient delivery vehicles to direct specific targeting of novel therapeutics without immunogenicity and adverse effects. In some embodiments, EVs, for example exosomes, may be pulsed with tumor peptides to make cell-free cancer vaccines.

In some embodiments, the disclosure contemplates a method of treating a patient comprising the steps of: a) inducing iPSC cells to produce megakaryocytes (MKs); b) culturing said MKs in a bioreactor for a sufficient time period, under conditions permissible for an admixture of PLCs and exosome production; c) collecting PLCs and exosomes produced by said MKs; d) concentrating said collected PLCs and exosomes; and e) administering said concentrated PLCs and exosomes to said patient, wherein said patient has a disorder or a disease that benefits from the treatment with such PLCs and exosomes.

In some embodiments, the disclosure contemplates a method of treating a patient comprising the steps of: a) inducing exosome producing progenitor cells (e.g., iPSC cells) to produce megakaryocytes (MKs); b) culturing said MKs in a bioreactor for a sufficient time period, under conditions permissible for PLCs and exosome production; c) isolating exosomes from the PLCs produced by the MKs; d) concentrating said isolated exosomes substantially pure from PLCs; and e) administering said concentrated exosomes to said patient, wherein said patient has a disorder or a disease that benefits from the treatment with such exosomes.

In some embodiments, the EV-based treatment may be administered prior to PLC-based treatment. In some embodiments, PLC-based treatment may be administered prior to EV-based treatment. In some embodiments, PLCs and EVs are administered as admixtures. Also contemplated are treatments in which admixtures comprising PLCs and EVs are administered followed by treatment regiments comprising essentially of EV or derivatives thereof or comprising essentially of PLCs or derivatives thereof-based treatment depending on a patient's need.

PLC and/or EV-Based Disease Targeting

This novel strategy takes advantage of several of the PLC and/or EV properties, such as, but not limited to, flexible morphology, cellular signaling, abundant of growth factors, ease of engineering receptors, ligand or antigens into the PLCs/EVs, abundant of endogenous receptors/ligands, and relocating to liver upon circulation for at least inducing the liver tolerance effect or clearance of the diseased molecules or toxins by the liver, to offer a unique opportunity to maximize therapeutic outcomes as well as minimizing side effects.

Some embodiments take advantage of the PLCs' and/or EVs' (or derivatives thereof) ability to communicate with a cell surface, cell protein, cell receptor or a cell ligand in a cell at a diseased location. In some embodiments, the PLCs' and/or EVs' (including any derivatives thereof) receptors or ligands may carry to a diseased location drug payload that upon PLC-cell or EV-cell interaction at the diseased location delivers the drug payloads at the diseased location, for example, through PLC and/or EV-based receptor-ligand interaction with a diseased cell (e.g., PLC-receptor binding to a ligand on a diseased cell and vice a versa). Thus, the PLC ligand-receptor or EV-ligand-receptor interaction properties can be manipulated to advantageously deliver drug payloads of the present disclosure because the targeting strategies are selective and specific. Considering that PLCs and EVs are relatively rich in receptors on their surface, relatively have a large surface area, a variety of drugs and in greater payloads may be engineered into, attached to or imbibed into the PLCs and/or EVs for delivery to a diseased location or to target diseased molecules.

In some embodiments, the PLCs and/or the EVs or derivatives thereof of the present disclosure can be generated in a device or a system that supports a biologically active environment e.g., bioreactors or fluidic devices. Bioreactors or fluidic devices could include, but is not limited to, shear stress, mechanical strain and pulsed electromagnetic field bioreactors, large-scale stirred tank bioreactors, automated bioreactors, rotating wall bioreactors (RWBs), and rocking motions as seen with wave bioreactors, organ-on-chip bioreactors. Other bioreactor configurations that enable continuous, perfusion operation such as packed bed bioreactors (PBBs), fluidized bed bioreactors (FBBs), or PBBs or FBBs including the use of microcarriers, CultiBag bioreactors, and membrane bioreactors such as hollow fiber bioreactors (HFBs) are also contemplated for generating the PLCs/EVs or derivatives thereof of the present disclosure. Operation of the bioreactors may require coupling with an internal or external cell retention device on a recycle line, by centrifugation, sedimentation, ultrasonic separation or microfiltration with spin-filters, alternating tangential flow (ATF) filtration or tangential flow filtration (TFF) or in vivo bioreactors, which are a pocket within the body into which biomaterials (e.g., PLCs or their derivatives or the progenitor cells form which they are derived from) are implanted at a site in need thereof and incubated for an extended period of time. Within these pockets (for example, bone tissue or muscle flap etc.), the grafts harness the regenerative capacity of the body to recover from a disease or an injury. Non-limiting examples of bioreactors are described, for example, in the co-filed application titled: Simultaneous Welding of Three Components To Form a Bioreactor or Filter Structure (U.S. Application No. 62/981,373) or elsewhere, for example tools and technologies (e.g., bioreactors or fluidic devices) disclosed in U.S. Pat. Nos. 9,795,965; 10,343,163; 9,763,984; 9,993,503; and 10,426,799; U.S. Patent Publication No. 20180334652; PCT Patent Application Nos. PCT/US2018/021354; PCT/US2019/012437; PCT/US2019/040021, and U.S. patent application Ser. No. 16/730,603, each of which is incorporated herein in their entirely by reference. Bioreactors or microfluidic devices known or unknown that can routinely generate the PLCs or derivatives are also contemplated for use in the present disclosure.

### Genetic Engineering

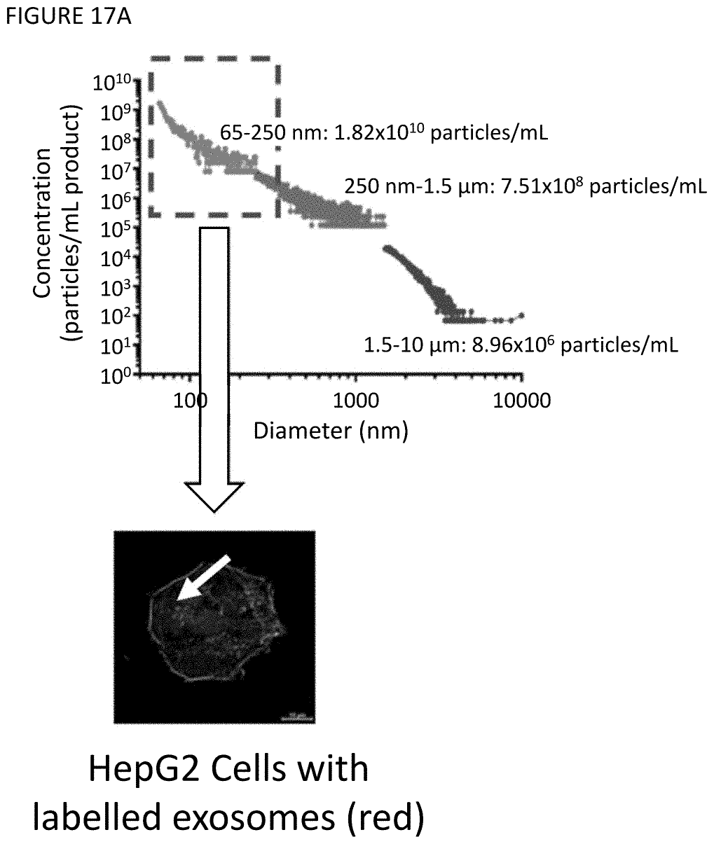

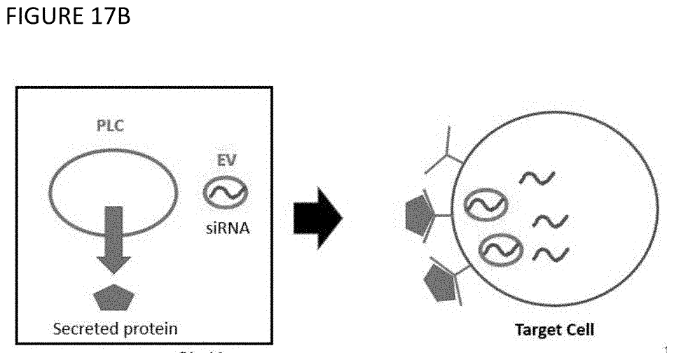

In some embodiments, the PLC and EV producing cells (e.g., iPSCs or Megakaryocytes) are genetically engineered to exogenously express a ligand, receptor or an antigen or to produce PLCs and/or EVs that deliver biologic or cytotoxic agents, such as but not limited to, antibodies (e.g., human or humanized antibodies), RNAs (e.g., siRNA, piRNAs, miRNAs and the like), cytokines (e.g., interferon, interleukin and the like), hormones (e.g., thyroid hormones, peptide hormones, amino acid derived hormones and the like) or growth factors (e.g., HGF, factor VIIa, FGF, EGF and the likes), discussed below in greater detail. In some embodiments, PLC and EV producing cells (e.g., iPSCs or Megakaryocytes) are genetically engineered to produce PLCs and/or EVs expressing two or more biologic or cytotoxic agents, such as RNAs (e.g., siRNA against a growth factor) and growth factors (e.g., HGF). For example, PLCs and/or EVs may express both a secreted protein growth factor and an inhibitory siRNA in the same cell. In this embodiment, iPSCs would be genetically engineered with constructs that would express both a growth factor (e.g., HGF) and an inhibitor siRNA (e.g., against TGF-beta mRNA). This iPSC clone would then be differentiated into Megakaryocytes and then put through the bioreactor. Out of the bioreactor comes a mixture of a broad range of cell sizes, from platelet size (1-5 micron) down to EVs size (50-2000 nanometers). All of the cells in the mixture contain both HGF protein and TGF-beta siRNA. When the patient is dosed, the cells go to the liver where the PLCs/EVs or derivatives thereof secrete HGF which activates hepatocyte growth and the siRNA goes inside the hepatocyte and knocks down the expression of TGF-beta, which is a pro-fibrotic cytokine. More than one growth factor and siRNA could also be engineered, FIG. 17 illustrates this concept. The PLC and the EV producing cells (e.g., iPSCs or Megakaryocytes) or the PLCs and/or EVs are genetically engineered by introducing into an isolated population of PLC and/or EV producing cells or the PLC or EV population one or more exogenous nucleic acid. In some embodiments, the nucleic acids of the present disclosure, i.e., nucleic acids encoding a protein of interest is operably linked to a regulatory element, can be stably inserted into isolated population of PLC and/or EV producing cells (e.g., iPSCs or Megakaryocytes) or the PLCs or EV as naked DNA or RNA or more commonly as part of a vector to facilitate manipulation of the nucleic acid. As used herein, the term nucleic acid refers to a nucleic acid molecule(s) (e.g., encoding one or more proteins), which is/are inserted by artifice into a cell and is stably integrated into the chromosomal genome of the cell or is stably maintained as an episome.

Nucleic acids can be introduced into the isolated population of PLC and/or EV producing cells (e.g., iPSCs or Megakaryocytes) or the PLC or EV or both by means of one or more viral vectors, such as but not limited to adenoviral vectors, adeno-associated viral vectors, herpes simplex viral vectors, vaccinia viral vectors, baculoviral vectors or retroviral vectors. There are many retroviral based vectors. For the present application, the term “retrovirus” includes: murine leukemia virus (MLV), human immunodeficiency virus (HIV), equine infectious anemia virus (EIAV), mouse mammary tumor virus (MMTV), Rous sarcoma virus (RSV), Fujinami sarcoma virus (Fussy), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), adenoviral vectors, adeno-associated virus (AAV), and Avian erythroblastosis virus (AEV) and all other retroviridiae including lentiviruses.

### General Structure of the Vectors

Lentiviral vectors are at the forefront of gene delivery systems for research and clinical applications. These vectors can efficiently transduce nondividing and dividing cells, to insert large genetic segment in the host chromatin, and to sustain stable long-term transgene expression. Like other retroviruses, lentiviruses have gag, pol and env genes flanked by two LTR (Long Terminal Repeat) sequences. Each of these genes encodes for numerous proteins which are initially expressed in the form of a single precursor polypeptide. The gag gene encodes for the internal structure proteins (capsids and nucleocapsid). The pol gene encodes for inverse transcriptase, integrase and protease. The env gene encodes for viral envelope glycoprotein. Furthermore, the lentivirus genome contains a cis-acting RRE (Rev Responsive Element) element responsible for exporting out of the nucleus of the viral genomic RNA which will be packaged. The LTR 5′ and 3′ sequences serve to promote the transcription and polyadenylation of the viral RNAs. The LTR contains all the other cis-acting sequences necessary for viral replication. Sequences necessary for the inverse transcription of the genome (linkage site of the RNAt primer) and for the encapsidation of viral RNA in particles (T site) are adjacent to the LTR 5′. If the sequences necessary for encapsidation (or for packaging retroviral RNA in the infectious virions) are absent from the viral genome, genomic RNA will not be actively packaged. Furthermore, the lentiviral genome comprises accessory genes such as vif, vpr, vpu, nef, TAT, REV etc. The construction of lentiviral vectors for gene transfer applications has been described, for example, in U.S. Pat. Nos. 5,665,577; 5,981,276; and 6,013,516 or in Patent Application Nos. EP 386 882; WO99/58701; and WO02/097104, incorporated herein by reference in their entireties. These vectors include a defective lentiviral genome, i.e., in which at least one of the gags, pol and env genes has been inactivated or deleted.

Lentivirus experiments can be performed using lentivirus vectors known to one of skill in the art. As a non-limiting example, lentivirus vectors such as but not limited to GCMV-MCS-IRES-eGFP and GCMV-MCS-IRES-dsRed can be used to deliver a transgene of interest. Both vectors are HIV1 strains that lack the structural viral genes gag, pol, env, rev, tat, vpr, vif, vpu, and nef. In addition, there is a partial deletion of the promoter/enhancer sequences within the 3′ LTR that renders the 5′ LTR/promoter self-inactivating following integration. The genes provided in trans for both vectors are the structural viral proteins Gag, Pol, Rev, and Tat (via plasmid Delta8.9) and the envelope protein VSV-G. These plasmids are introduced into the PLCs by co-transfection, and transiently express the different viral proteins required to generate viral particles. The potential for generating wild type or pathogenic lentivirus is extremely low because it would require multiple recombination events amongst three plasmids. In addition, the virulence factors (vpr, vif, vpu, and nef) have been completely deleted from both vectors. Non-limiting examples of lentiviral vectors are described in the example section of the application.

In some embodiments, the isolated population of PLC and EV producing cells (e.g., iPSCs or Megakaryocytes) or the PLCs and/or EVs are genetically engineered by introducing into an isolated population of PLC or EV producing cells (e.g., iPSCs or Megakaryocytes) or the PLCs and/or EVs or both a first transgene comprising an inducible promoter and nucleotide sequences encoding one or more exogenous proteins for their transcription under the control of the inducible promoter. Alternatively, a second transgene is introduced into the same population of PLCs and/or EVs or their progenitor cells, the second transgene comprising a constitutive promoter and a nucleotide sequence encoding a transcription factor for the constitutive expression of the transcription factor under the control of the constitutive promoter, the transcription factor specific for binding to the inducible promoter in the first transgene thereby inducing transcription of the proteins from the coding sequences in the first transgene.

In the case of an antibody or a fragment thereof that is encoded by a transgene or an antibody or a fragment thereof is conjugated to the PLCs and/or EVs, the antibody or the fragment thereof is preferably a cell binding agent, i.e., the antibody or the fragment thereof binds to one or more receptors or ligands or to any other binding element on a cell for which the antibody is specific for as commonly known to one of skill in the art. For example, the antibody anti-CTLA4 is a hIgG1 antibody that binds specifically to the CTLA4 receptors and can be used if the target T cells expressing CTLA4.

The cell-binding agent may be any compound that can bind a cell, either in a specific or non-specific manner. Generally, these can be antibodies (especially monoclonal antibodies and antibody fragments), interferons, lymphokines, hormones, growth factors (e.g., HGF), vitamins, nutrient-transport molecules (such as transferrin), blood-coagulation factor VIIa, or any other cell-binding molecule or substance.

In some embodiments, the exogenous genetic material may be selected from, but not limited to, siRNA, shRNA, ceDNA, DNA, in one or in separate vectors with independent inducible (e.g., Tetracycline (Tet) Inducible Expression) or constitutive promoters or a combination thereof. The transgene of the present disclosure, i.e., a nucleic acid encoding a protein of interest is operably linked to a constitutive or an inducible regulatory element that can be stably inserted into the PLCs and/or EVs as naked DNA or more commonly as part of a vector to facilitate manipulation of the transgene(s). Viral vectors are well known to one skilled of the art and deposits of such vectors are commercially available at https://www.addgene.org/.

### Bioconjugates

In some embodiments, PLCs or the EVs or derivatives thereof of the present disclosure may also be used as a bioconjugate. PLC or EV bioconjugates can be (i) a linker-based bioconjugate, where one or more of a PLC or EV receptor protein or ligand or a molecule on the PLC or EV cell surface is linked to a cytotoxic agent via a linker; (ii) the PLC and/or EV bioconjugate can be a linker free bioconjugate, where one or more of cytotoxic agents are directly conjugated to a receptor protein or ligand or a molecule on the PLC and/or EV cell surface without the aid of a linker; or (iii) PLCs and/or EV may imbibe the cytotoxic agents.

### Linker Based Bioconjugates

This novel strategy takes advantage of PLC and/or the EV properties, such as, abundant conjugatable surface receptors or ligands, flexible cellular morphology, cellular signaling, and metabolism, to offer a unique opportunity to maximize therapeutic outcomes as well as minimizing side effects.

In some embodiments, the non-naturally occurring PLCs or derivatives thereof or the EVs may be bioconjugated to a cytotoxic agent with the aid of a linker. When a linker is used to link a cytotoxic agent to the PLCs and/or EVs the conjugates have the configuration (A)-(L)-(C); wherein, (A) is non-naturally occurring PLCs and/or EVs described herein; (L) is a linker; and (C) is a cytotoxic agent; and wherein the linker (L) links (A) to (C).

As an example, one or more of PLC or EV receptor proteins (transmembrane or surface) can be modified by reacting a bifunctional crosslinking reagent with the one or more of PLC or EV receptor proteins (for example PLC-based receptor CD63 or EV based receptor CD9), thereby resulting in the covalent attachment of a linker molecule to the PLCs and/or EVs. As used herein, a “bifunctional crosslinking reagent” is any chemical moiety that covalently links a cell-binding agent to a drug, such as the drugs described herein. In some embodiments, a portion of the linking moiety is provided by the drug. In this respect, the drug comprises a linking moiety that is part of a larger linker molecule that is used to join the cell-binding agent to the drug. For example, to form the maytansinoid based conjugate, the side chain at the C-3 hydroxyl group of maytansine is modified to have a free sulfhydryl group (SH). This thiolated form of maytansine can react with a modified cell-binding agent to form a conjugate. Therefore, the final linker is assembled from two components, one of which is provided by the crosslinking reagent, while the other is provided by the side chain from maytansine.

In some embodiments, the PLC or EV receptor protein is linked to the drug via a non-cleavable bond through the intermediacy of a PEG spacer. Suitable crosslinking reagents comprising hydrophilic PEG chains that form linkers between a drug and the PLC or EV receptor protein are also well known in the art or are commercially available (for example from Quanta Biodesign, Powell, Ohio). Suitable PEG-containing crosslinkers can also be synthesized from commercially available PEGs themselves using standard synthetic chemistry techniques known to one skilled in the art. The drugs can be reacted with bifunctional PEG-containing cross linkers to give compounds of the following formula, Z—X₁—(—CH₂—CH₂—O—)ₙ—Yₚ-D, by methods described in detail in U.S. Patent Publication No. 20090274713 and in WO2009/0134976, which can then react with the cell binding agent to provide a conjugate. Alternatively, the cell binding can be modified with the bifunctional PEG crosslinker to introduce a thiol-reactive group (such as a maleimide or haloacetamide) which can then be treated with a thiol-containing maytansinoid to provide a conjugate. In some embodiments, the cell binding can be modified with the bifunctional PEG crosslinker to introduce a thiol moiety which can then be treated with a thiol-reactive maytansinoid (such as a maytansinoid bearing a maleimide or haloacetamide), to provide a conjugate.

### Infusion of Cytotoxic Agents

This novel strategy takes advantage of PLC's and EV properties, such as, flexible morphology, large surface area, to offer a unique opportunity to maximize therapeutic outcomes as well as minimizing side effects. This novel strategy also takes advantage of PLC's and/or EV's unique property to store secretory granules, which remain stored in the PLCs and/or EVs until PLCs and/or EVs trigger their release.

Thus, some embodiments take the advantage of the cell membrane permeability of the PLCs or MVs or exosomes (collectively referred throughout as EVs) in hypotonic solution, which enables the entrapment of drugs, biomacromolecules, and nanoparticles into PLCs' or MVs' or exosomes' cavities generally reserved for storing PLC or EV-derived secretory granules. The principle for infusion of cytotoxic agents into PLCs or derivatives thereof or EVs is because the absence of a superfluous membrane on the PLCs and/or EVs allows PLCs and/or EVs to accommodate additional volume by changing the shape, for example, from biconcave to spherical. Several hypotonic hemolysis techniques have been generated, such as hypotonic dialysis, hypotonic dilution, and hypotonic pre-swelling. Hypotonic dialysis is predominantly applied in encapsulating enzymes, proteins, and contrast agents due to its relative ease of use, ability to preserving characteristics of the PLCs or their derivatives or EVs and high encapsulation rate. In the process, PLCs or their derivatives or EVs may be prepared in a dialysis tube and immersed in a hypotonic buffer for a few hours under gentle stirring. Nucleic acids (e.g., RNAs or DNAs) or proteins (e.g., antibodies or fragments thereof or growth factors), as therapeutic agents, may be loaded into PLCs and/or EVs via hypotonic dialysis and achieve between 20% to 90% encapsulation after 2-28 hours incubation. Nucleic acid PLCs or derivatives thereof or EVs may undergo opsonization with ZnCl₂ and bis-sulfosuccynimidil-suberate treatment, and then specifically could be used to target a cell or a tissue, such as a tumor cell or T-cells or macrophages, at a second location. In this manner, nucleic acids or proteins may effectively be delivered and result in production of enzymatic activities or physiological reactions to inhibit protein expression, for example PLCs and/or EVs may induce nitric oxide synthesis thereby blocking recruitment of bone marrow derived platelets at tumor sites in a tumor microenvironment thereby preventing tumor metastasis. It is well known that upon tumor cell arrival in the blood, tumor cells immediately activate platelets to form a permissive microenvironment. Platelets protect tumor cells from shear forces and assault of NK cells, recruit myeloid cells by secretion of chemokines, and mediate an arrest of the tumor cell platelet embolus at the vascular wall. Subsequently, platelet-derived growth factors confer a mesenchymal-like phenotype to tumor cells and open the capillary endothelium to expedite extravasation in distant organs. Finally, platelet-secreted growth factors stimulate tumor cell proliferation to micro metastatic foci. Thus, in some embodiments the PLCs or derivatives or EVs of the present disclosure could act as a decoy to fool metastasizing tumor cells into communicating with payload bearing PLCs and/or EVs rather than endogenous platelets thereby limiting the tumor metastasizing role played by endogenous platelets.

### Receptors

PLC or EV receptors, whether cell surface or transmembrane or exogenously engineered into the PLCs/EVs or derivatives thereof that can be used in some embodiments are inclusive of, but not limited to, cell-surface receptors or transmembrane receptors, ion channel-linked receptors, G-protein-coupled receptors, enzyme-linked receptors or internal receptors or a combination thereof. Non-limiting examples of receptors are P2Y1, P2Y12, PAR1, PAR4, Tpa, PAF receptors, PGE2 receptor (EP3), Lysophosphatidic acid receptor, Chemokine receptors, V1a vasopressin receptor, A2a adenosine receptor, b2 adrenergic receptor, Serotonin receptor, Dopamine receptor, P2X1, c-Mp1, Insulin receptor, PDGF receptor, Leptin receptor, GPVI, CD148, CLEC-2, Eph receptor, Axl/Tyro3/Mer, P-selectin, TSSC6, CD151 CD36, TLT-1, PEAR1, VPAC1, PECAM-1, G6B-b, PGI2 receptor (IP), PGD2 receptor, PGE2 receptor (EP4), GPIb-IX-V complex, Alix, Tsg101, Hsc70, CD63 CD81 CD9, flotillin 1, HSP70 or a modified version thereof.

### Receptor Families

In some embodiments, of one skilled in the art may easily replace or supplement one receptor with another belonging to the same or different families of PLC or EV receptors, which could be engineered into PLCs and/or EVs if desired. A skilled artisan can pick one or more receptors from the Leucine-rich repeat family, Ig superfamily, Integrins, Tyrosine phosphatase receptor, C-type lectin receptor, G protein-coupled receptors, Ion channel, Tyrosine kinase receptor, Cytokine, C-type lectin receptor family, tetraspanins, Class B scavenger receptor, Multiple EGF-like domain protein, transmembrane 4 superfamily, as these families are generally inclusive of receptors on the PLCs and/or EVs.

### Ligands

Several ligands specifically bind to receptors on PLCs and/or EVs. In the alternative, ligands can be genetically engineered into the PLCs and/or EVs to bind to receptors or antigens on a diseases cell. The ligands that can be used in the present disclosure are mostly proteins but are also inclusive of hydrophobic molecules like steroids, or gases (e.g., nitric oxide). For example, Willebrand factor (VWF) interacts with the PLC receptors, glycoprotein (GP) Ib-IX-V and αIIBβ3 integrin, to promote primary platelet adhesion and aggregation following vessel injury. The ability of VWF to bind to PLC receptor GPIb-IX-V provides a target for the treatment of diseases related to arterial and venous pathological thrombosis. Likewise, CD36 receptors on the PLCs recognize at least three classes of ligand: modified phospholipids, a subset of proteins containing a structural domain termed the thrombospondin type I repeat (TSR), and free fatty acids. Studies have shown that loss of CD36 confers substantial protection against atherosclerosis. In contrast, CD36-mediated anti-angiogenesis is caused by its ability to activate a specific signaling cascade that results in diversion of a proangiogenic response to an apoptotic response. Thus, the CD36 receptors in the PLCs can be genetically manipulated or chemically modified to influence the CD36 receptor's interaction with the ligands (e.g., TSP1, oxLDL, VLDL, oxPL). Here a patient suffering from a CD36 related disorder can benefit by manipulating the CD36 receptor or the ligands that bind to it to provide a therapeutic relief (e.g., protection from atherosclerosis).

Ligands that can be genetically engineered, imbibed or bioconjugated to a cytotoxic agent, such as, proteins, polypeptides, nucleic acid molecules or small molecule drugs, include, but are not limited to, vWf, thrombin, FXI, FXII, P-selectin, HK, Mac-1, TSP-1, Collagen, laminin, Fibronectin, Vitronectin, fibrinogen, vWf, osteopontin, fibrin, vWf, TSP-1, Podoplanin, ADP, Thrombin, Thromboxane, 1-O-alkyl-2-acetyl-sn-glycero-3-phosphocholine, PGE2, Lysophosphatidic acid, Chemokines, Vasopressin, Adenosine, Epinephrine, Serotonin (5-hydroxytryptamin), Dopamine, ATP, TPO, Insulin, PDGF, Leptin, Ephrin, Gas-6, PSGL-1, GPIb, TF, TSP1, oxLDL, VLDL, oxPL, collagen type V, Fibrinogen, PACAP, PECAM-1, collagen, glycosaminoglycans, PGI2, PGD2.

### Linkers

Linkers are well characterized in the literature and linker binding technologies are well known to one of skill in the art. To advance some embodiments disclosed herein, linkers may be selected from a cleavable linker, a non-cleavable linker, a hydrophilic linker, and a dicarboxylic acid-based linker. For example, bifunctional crosslinking agents that enable linkage via a thioether bond include N-succinimidyl-4-(N-maleimidomethyl)-cyclohexane-1-carboxylate (SMCC) to introduce maleimido groups, or with N-succinimidyl-4-(iodoacetyl)-aminobenzoate (SIAB) to introduce iodoacetyl groups. Other bifunctional crosslinking agents that introduce maleimido groups or haloacetyl groups onto a cell binding agent are well known in the art (see U.S. Patent Application Publication Nos. 2008/0050310 and 2005/0169933, available from Pierce Biotechnology Inc. P.O. Box 117, Rockland, Ill. 61105, USA) and include, but are not limited to, bis-maleimidopolyethyleneglycol (BMPEO), BM(PEO)₂, BM(PEO)₃, N-(3-maleimidopropyloxy)succinimide ester (BMPS), gamma-maleimidobutyric acid N-succinimidyl ester (GMBS), epsilon-maleimidocaproic acid N-hydroxysuccinimide ester (EMCS), 5-maleimidovaleric acid NHS, HBVS, N-succinimidyl-4-(N-maleimidomethyl)-cyclohexane-1-carboxy-(6-amidocaproa-te), which is a “long chain” analog of SMCC (LC-SMCC), m-maleimidobenzoyl-N-hydroxysuccinimide ester (MBS), 4-(4-N-maleimidophenyl)-butyric acid hydrazide or HCl salt (MPBH), N-succinimidyl 3-(bromoacetamido)propionate (SBAP), N-succinimidyl iodoacetate (SIA), kappa-maleimidoundecanoic acid N-succinimidyl ester (KMUA), N-succinimidyl 4-(p-maleimidophenyl)-butyrate (SMPB), succinimidyl-6-(3-maleimidopropionamido)hexanoate (SMPH), succinimidyl-(4-vinyl sulfonyl)benzoate (SVSB), dithiobis-maleimidoethane (DTME), 1,4-bis-maleimidobutane (BMB), 1,4 bismaleimidyl-2,3-dihydroxybutane (BMDB), bis-maleimidohexane (BMH), bis-maleimidoethane (BMOE), sulfosuccinimidyl 4-(N-maleimido-methyl)cyclohexane-1-carboxylate (sulfo-SMCC), sulfosuccinimidyl(4-iodo-acetyl)aminobenzoate (sulfo-SIAB), m-maleimidobenzoyl-N-hydroxysulfosuccinimide ester (sulfo-MBS), N-(gamma-maleimidobutryloxy)sulfosuccinimde ester (sulfo-GMBS), N-(.epsilon.-maleimidocaproyloxy)sulfosuccimido ester (sulfo-EMCS), N-(.kappa.-maleimidoundecanoyloxy)sulfosuccinimide ester (sulfo-KMUS), and sulfosuccinimidyl 4-(p-maleimidophenyl)butyrate (sulfo-SMPB).

Heterobifunctional crosslinking agents are bifunctional crosslinking agents having two different reactive groups. Heterobifunctional crosslinking agents containing both an amine-reactive N-hydroxysuccinimide group (NHS group) and a carbonyl-reactive hydrazine group can also be used to link the cytotoxic compounds described herein with a cell-binding agent (e.g., PLC). Examples of such commercially available heterobifunctional crosslinking agents include succinimidyl 6-hydrazinonicotinamide acetone hydrazone (SANH), succinimidyl 4-hydrazidoterephthalate hydrochloride (SHTH) and succinimidyl hydrazinium nicotinate hydrochloride (SHNH). Conjugates bearing an acid-labile linkage can also be prepared using a hydrazine-bearing benzodiazepine derivative of the present disclosure. Examples of bifunctional crosslinking agents that can be used include succinimidyl-p-formyl benzoate (SFB) and succinimidyl-p-formylphenoxyacetate (SFPA).

Bifunctional crosslinking agents that enable the linkage of cell binding agents with cytotoxic compounds via disulfide bonds are known in the art and include N-succinimidyl-3-(2-pyridyldithio)propionate (SPDP), N-succinimidyl-4-(2-pyridyldithio)pentanoate (SPP), N-succinimidyl-4-(2-pyridyldithio)butanoate (SPDB), N-succinimidyl-4-(2-pyridyldithio)2-sulfo butanoate (sulfo-SPDB) to introduce dithiopyridyl groups. Other bifunctional crosslinking agents that can be used to introduce disulfide groups are known in the art and are disclosed in U.S. Pat. Nos. 6,913,748, 6,716,821, 8,236,319 and 9,150,649, all of which are incorporated herein by reference. Alternatively, crosslinking agents such as 2-iminothiolane, homocysteine thiolactone or S-acetylsuccinic anhydride that introduce thiol groups can also be used. Any of the linkers commonly known to one of the skill in the art could be used to conjugate the PLCs/EVs of the present disclosure.

### Cytotoxic Agents-Biologics

Cytotoxic agents, genetically engineered, imbibed or bioconjugated into the PLCs/EVs or derivatives thereof can be selected from one or more of proteins, antigens, antibodies or fragments thereof, growth factors, cytokines, hormones or nucleic acid, such as DNA or RNA. Antigens and antibodies or fragments thereof, being proteinaceous in nature, provide several advantages for antibody-based therapeutic payload delivery. For example, PLCs and/or EVs or derivatives thereof or the PLC or EV-producing progenitor cells can be genetically engineered to produce the antibodies or fragments thereof, growth factors, or RNAs as disclosed herein. For conjugation of an antibody or fragments thereof to the PLCs or derivative thereof or to the EVs, one or more amino acids of an antibody or a fragment thereof can be directly bioconjugated to PLC or EV cell surface or transmembrane receptor protein without the need of a chemical linking agent. Further, antibodies or fragments thereof are capable of binding to antigens on PLC or EV receptors. Lastly, antibodies can be attached to PLCs and/or EVs via linkers, as discussed in the foregoing. In a non-limiting example, for example, PLCs and/or EVs or PLC or EV producing progenitor cells can be genetically engineered in a lentivirus-based vector to produce ipilimumab, a monoclonal antibody that works to activate the immune system by targeting CTLA-4, a protein receptor that downregulates the immune system. In some embodiments, ipilimumab may be bioconjugated to PLC or EV receptor proteins through protein-protein conjugation or it may be conjugated via maleimide cross-linking reaction to free thiols which allows for stable conjugation to the PLC or EV surface protein having a modified thiol group or via a PLC or EV cell surface or a modified PLC or EV cell surface or a transmembrane protein.

Exemplary cytotoxic agents such as antigens, antibodies, hormones, cytokines, growth factors or nucleic acids or a combination thereof (e.g., an antibody and a growth factor and/or siRNA against a growth factor) that may be genetically engineered into PLCs and/or EVs or PLC or EV progenitor cells in, for example, a lentivirus based vector, or bioconjugated to the PLCs and/or EVs, include molecules such as renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor vmc, factor IX, tissue factor (TF), and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin, such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors; protein A or D; rheumatoid factors; a neurotrophic factor such as bone-derived neurotrophic factor (BDNF), neurotrophin-3, -4, -5, or -6 (NT-3, NT4, NT-5, or NT-6), or a nerve growth factor such as NGF-beta; platelet-derived growth factor (PDGF); fibroblast growth factor such as aFGF and bFGF; fibroblast growth factor receptor 2 (FGFR2), epidermal growth factor (EGF); transforming growth factor (TGF) such as TGF-alpha and TGF-beta, including TGF-betal, TGF-beta2, TGF-beta3, TGF-beta4, or TGF-beta5; bone morphogenetic protein (BMP), including BMP1, BMP6, BMP7, and BMP-receptor 2; insulin-like growth factor-I and -II (IGF-I and IGF-II); des(1-3)-IGF-I (brain IGF-I), insulin-like growth factor binding proteins, hepatocyte growth factor (HGF), EpCAM, GD3, FLT3, PSMA, PSCA, MUC1, MUC16, STEAP, CEA, TENB2, EphA receptors, EphB receptors, folate receptor, FOLR1, mesothelin, cripto, alphavbeta6, integrins, VEGF, VEGFR, EGFR, tarnsferrin receptor, IRTA1, IRTA2, IRTA3, IRTA4, IRTA5; CD proteins such as CD2 CD3 CD4 CD5 CD6 CD8, CD11 CD14 CD19 CD20 CD21 CD22 CD25 CD26 CD28 CD30 CD33 CD36 CD37 CD38, CD40 CD44 CD52 CD55 CD56 CD59 CD70 CD79 CD80. CD81 CD103 CD105 CD134 CD137, CD138 CD152, IFN gamma TNF alpha, IFN alpha, GM-CSF, IL-3 or an antibody which binds to one or more tumor-associated antigens or cell-surface receptors; erythropoietin; osteoinductive factors; immunotoxins; a bone morphogenetic protein (BMP); an interferon, such as interferon-alpha, -beta, and -gamma; colony stimulating factors (CSFs), e.g., M-CSF, GM-CSF, and G-CSF; interleukins (ILs), e.g., IL-2, IL-6, IL-12, IL-23, IL-12/23 p40, IL-17, IL-15, IL-21, IL-1a, IL-1b, IL-18, IL-8, IL-4, IL-3, and IL-5; superoxide dismutase; T-cell receptors; surface membrane proteins; decay accelerating factor; viral antigen such as, for example, a portion of the HIV envelope; transport proteins; homing receptors; addressins; regulatory proteins; integrins, such as CD11a, CD11b, CD11c, CD18, an ICAM, VLA-4 and VCAM; a tumor associated antigen such as HER2, HER3 or HER4 receptor; endoglin, c-Met, c-kit, 1GF1R, PSGR, NGEP, PSMA, PSCA, LGRS, B7H4, TAG72 (tumor-associated glycoprotein 72) and fragments of any of the above-listed polypeptides.

Examples of antibodies or fragments thereof that can be expressed as a transgene in PLCs and/or EVs or in PLC or EV-producing progenitor cells in a vector (e.g., a lentivirus based vector) or that can be linked to the PLCs and/or EVs include, but are not limited to, anti-PD-L1 antibodies, abciximab (Reopro), adalimumab (Humira, Amjevita), alefacept (Amevive), alemtuzumab (Campath), basiliximab (Simulect), belimumab (Benlysta), bezlotoxumab (Zinplava), canakinumab (Ilaris), certolizumab pegol (Cimzia), cetuximab (Erbitux), daclizumab (Zenapax, Zinbryta), denosumab (Prolia, Xgeva), efalizumab (Raptiva), golimumab (Simponi, Simponi Aria), inflectra (Remicade), ipilimumab (Yervoy), ixekizumab (Taltz), natalizumab (Tysabri), nivolumab (Opdivo), olaratumab (Lartruvo), omalizumab (Xolair), palivizumab (Synagis), panitumumab (Vectibix), pembrolizumab (Keytruda), rituximab (Rituxan), tocilizumab (Actemra), trastuzumab (Herceptin), secukinumab (Cosentyx), ranibizumab, abciximab, raxibacumab, caplacizumab, infliximab, bevacizumab, dabigatran, Idarucizumab, or ustekinumab (Stelara) or a combination thereof. Furthermore, the antibodies may be selected from anti-estrogen receptor antibody, anti-progesterone receptor antibody, anti-p53 antibody, anti-EGFR antibody, anti-cathepsin D antibody, anti-Bcl-2 antibody, anti-E-cadherin antibody, anti-CA125 antibody, anti-CA15-3 antibody, anti-CA19-9 antibody, anti-c-erbB-2 antibody, anti-P-glycoprotein antibody, anti-CEA antibody, anti-retinoblastoma protein antibody, anti-ras oncoprotein antibody, anti-Lewis X antibody, anti-Ki-67 antibody, anti-PCNA antibody, anti-CD3 antibody, anti-CD4 antibody, anti-CD5 antibody, anti-CD7 antibody, anti-CD8 antibody, anti-CD9/p24 antibody, anti-CD1-antibody, anti-CD11c antibody, anti-CD13 antibody, anti-CD14 antibody, anti-CD15 antibody, anti-CD19 antibody, anti-CD20 antibody, anti-CD22 antibody, anti-CD23 antibody, anti-CD30 antibody, anti-CD31 antibody, anti-CD33 antibody, anti-CD34 antibody, anti-CD35 antibody, anti-CD38 antibody, anti-CD39 antibody, anti-CD41 antibody, anti-LCA/CD45 antibody, anti-CD45RO antibody, anti-CD45RA antibody, anti-CD71 antibody, anti-CD95/F as antibody, anti-CD99 antibody, anti-CD100 antibody, anti-S-100 antibody, anti-CD106 antibody, anti-ubiquitin antibody, anti-c-myc antibody, anti-cytokeratin antibody, anti-lambda light chains antibody, anti-melanosomes antibody, anti-prostate specific antigen antibody, anti-tau antigen antibody, anti-fibrin antibody, anti-keratins antibody, and anti-Tn-antigen antibody.

Monoclonal antibody techniques allow to produce extremely specific cytotoxic agent in the form of specific monoclonal antibodies. Particularly well known in the art are techniques for creating monoclonal antibodies produced by immunizing mice, rats, hamsters or any other mammal with the antigen of interest such as the intact target cell, antigens isolated from the target cell, whole virus, attenuated whole virus, and viral proteins such as viral coat proteins. Sensitized human cells can also be used. Another method of creating monoclonal antibodies is the use of phage libraries of scFv (single chain variable region), specifically human scFv (see e.g., Griffiths et al., U.S. Pat. Nos. 5,885,793 and 5,969,108; McCafferty et al., WO 92/01047; Liming et al., WO 99/06587). In addition, resurfaced antibodies disclosed in U.S. Pat. No. 5,639,641 may also be used, as may chimeric antibodies and humanized antibodies. Selection of the appropriate cytotoxic agent is a matter of choice that depends upon the cell population that is to be targeted, but in general human monoclonal antibodies or fragments thereof are preferred if an appropriate one is available.

### Cytotoxic Agents-Drugs

The cytotoxic agents for the use in the embodiments of the present disclosure is a matter of choice for one of skill in the art depending on a disease which needs to be treated taking advantage of property of the PLCs or the EVs or derivatives thereof to reach that target through the blood circulatory system. These cytotoxic agents genetically engineered, imbibed or bioconjugated into the PLCs/EVs or derivative thereof may be selected from, but not limited to, an immunoinflammatory drug, a metabolic drug, neoplastic drug, a drug for curing autoimmune disease (e.g., immune thrombocytopenia (ITP), Myasthenia gravis, acquired thrombotic thrombocytopenic purpura (aTTP), Membranous Nephropathy, Neuromyelitis Optica Spectrum Disorder, N-methyl D-aspartate (NMDA) receptor (NMDAR) Encephalitis) or any drug which the PLCs and/or EVs or derivatives thereof of the present disclosure can deliver to a diseased target in need of that drug in human body. Once a symptom or a disease is identified appropriate drugs can be used as cytotoxic agents to cure that disorder. Drugs for delivery through the PLCs and/or EVs or derivatives thereof can be selected from references such as Merck manual or by referring to Index to Drug-Specific Information on US Food & Drug Administration website: https://www.fda.gov/drugs/postmarket-drug-safety-information-patients-and-providers/index-drug-specific-information, incorporated herein by reference.

In some embodiments, the present disclosure provides a method for treating a cell proliferative disorder in a patient comprising administering to said patient a therapeutically effective amount of a pharmaceutical composition comprising non-naturally occurring PLCs, or EVs or derivatives thereof (e.g., PLCs and/or EVs exogenously expressing Factor VIIa or IL-2 or any other cytokine or growth factor or inhibitors thereof). The cell proliferative disorder can be selected from the group consisting of adrenal cortex hyperplasia (Cushing's disease), congenital adrenal hyperplasia, endometrial hyperplasia, benign prostatic hyperplasia, breast hyperplasia, intimal hyperplasia, focal epithelial hyperplasia (Heck's disease), sebaceous hyperplasia, compensatory liver hyperplasia, and any other cell proliferation disease, besides neoplasia.

In some embodiments, the present disclosure also provides administering to an individual an effective amount of one or more therapeutic agents, such as a chemotherapeutic agent or an immunomodulatory drug, which could be same or different from a first therapeutic agent. The first and/or the second therapeutic agents (or a repeat uses thereof) could be selected from one or more of imanitib, gefitinib, erlotinib, sunitinib, lapatinib, nilotinib, sorafenib, temsirolimus, sverolimus, pazopanib, crizotinib, ruxolitinib, axitinib, bosutinib, cabozantinib, ponatinib, regorafenib, ibrutinib, trametinib, perifosine, bortezomib, carfilzomib, batimastat, ganetespib, NVP-AUY922, obatoclax or navitoclax, thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL™); beta-lapachone; lapachol; colchicines; betulinic acid; a camptothecin (including the synthetic analogue topotecan (HYCAMTIN™), CPT-11 (irinotecan, CAMPTOSAR™), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gammal I and calicheamicin omegall; CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN™, morpholino-doxorubicin, cyanomorpholino-doxorubicin, 2-pyrrolino-doxorubicin, doxorubicin HCl liposome injection (DOXIL™), liposomal doxorubicin TLC D-99 (MYOCET™), peglylated liposomal doxorubicin (CAELYX™), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR™), tegafur (UFTORAL™), capecitabine (XELODA™), an epothilone, and 5-fluorouracil (5-FU); combretastatin; folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK™ polysaccharide complex (JHS Natural Products, Eugene, Oreg.); razoxane; rhizoxin; sizofuran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE™, FILDESIN™); dacarbazine; mannomustine; mitobronitol; mitolactol; pipobroman; gacytosine; arabinoside (“Ara-C”); thiotepa; taxoid, e.g., paclitaxel (TAXOL™), Bristol-Myers Squibb Oncology, Princeton, N.J.), paclitaxel formulated in albumin-engineered nanoparticle (ABRAXANE™), and docetaxel (TAXOTERE™), Rhome-Poulene Rorer, Antony, France); chloranbucil; 6-thioguanine; mercaptopurine; methotrexate; platinum agents such as cisplatin, oxaliplatin (e.g., ELOXATIN™), and carboplatin; vincas, which prevent tubulin polymerization from forming microtubules, including vinblastine (VELBAN™), vincristine (ONCOVIN™), vindesine (ELDISINE™, FILDESIN™), and vinorelbine (NAVELBINE™); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as retinoic acid, including bexarotene (TARGRETIN™); bisphosphonates such as clodronate (for example, BONEFOS™ or OSTAC™), etidronate (DIDROCAL™), NE-58095, zoledronic acid/zoledronate (ZOMETA™), alendronate (FOSAMAX™), pamidronate (AREDIA™), tiludronate (SKELID™), or risedronate (ACTONEL™); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R) (e.g., erlotinib (Tarceva™)); and VEGF-A that reduce cell proliferation; vaccines such as THERATOPE™ vaccine and gene therapy vaccines, for example, ALLOVECTIN™ vaccine, LEUVECTIN™ vaccine, and VAXID™ vaccine; topoisomerase 1 inhibitor (e.g., LURTOTECAN™); rmRH (e.g., ABARELIX™); BAY439006 (sorafenib; Bayer); SU-11248 (sunitinib, SUTENT™, Pfizer); perifosine, COX-2 inhibitor (e.g., celecoxib or etoricoxib), proteosome inhibitor (e.g., PS341); bortezomib (VELCADE™); CCI-779; tipifarnib (R11577); orafenib, ABT510; Bcl-2 inhibitor such as oblimersen sodium (GENASENSE™); pixantrone; EGFR inhibitors; tyrosine kinase inhibitors; serine-threonine kinase inhibitors such as rapamycin (sirolimus, RAPAMUNE™); farnesyltransferase inhibitors such as lonafarnib (SCH 6636, SARASAR); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin, and pharmaceutically acceptable salts, acids or derivatives of any of the above; as well as combinations of two or more of the above.

Chemotherapeutic agents as defined herein include “anti-hormonal agents” or “endocrine therapeutics” which act to regulate, reduce, block, or inhibit the effects of hormones that can promote the growth of cancer. They may be hormones themselves, including, but not limited to: anti-estrogens and selective estrogen receptor modulators (SERMs), including, for example, tamoxifen (including NOLVADEX™ tamoxifen), raloxifene, droloxifene, 4-hydroxytamoxifen, trioxifene, keoxifene, LY117018, onapristone, toremifene; aromatase inhibitors that inhibit the enzyme aromatase, which regulates estrogen production in the adrenal glands, such as, for example, 4(5)-imidazoles, aminoglutethimide, MEGASE™ megestrol acetate, AROMASIN™ exemestane, formestanie, fadrozole, RIVISOR™ vorozole, FEMARA™ letrozole, and ARIMIDEX™ anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in aberrant cell proliferation, such as, for example, PKC-alpha, Raf and H-Ras; ribozymes such as a VEGF expression inhibitor (e.g., ANGIOZYME™ ribozyme) and a HER2 expression inhibitor; vaccines such as gene therapy vaccines, for example, ALLOVECTIN™ vaccine, LEUVECTIN™ vaccine, and VAXID™ vaccine; PROLEUKIN™ r1L-2; LURTOTECAN™ topoisomerase 1 inhibitor; ABARELIX™ rmRH; Vinorelbine and Esperamicins, and pharmaceutically acceptable salts, acids or derivatives of any of the above; as well as combinations of two or more of the above.

### Cytotoxic Agents—Immunomodulatory Drugs

In some embodiments, the present disclosure also encompasses immunomodulatory drugs for use with the PLCs or the EVs derivatives thereof of the present disclosure. The term “immunomodulatory drug” refers to a class of drugs that modifies the immune system response or the functioning of the immune system, such as by the stimulation of antibody formation and/or the inhibition of peripheral blood cell activity, and include, but are not limited to, thalidomide (a-N-phthalimido-glutarimide) and its analogues, REVLIMID™ (lenalidomide), ACTI-MID™ (pomalidomide), OTEZLA™ (apremilast), and pharmaceutically acceptable salts or acids thereof.

### Disease or Disorders

Various diseases and disorders can be treated by the PLCs or derivatives of or the EV or derivatives thereof the present disclosure because of their ability to be administered locally or to circulate through the blood system and reaching a diseased target with ease as well as their ability to carry greater amount of drug payloads than conventional drug delivery means which uses the same methodology but without the PLCs and/or EVs (e.g., ADCs). These diseases or disorders are inclusive of but not limited to one or more of an immunoinflammatory disorder, a metabolic disorder, neoplastic disorder, autoimmune disorder, liver disease, viral or bacterial-induced diseases or infections, or any disorder where the PLCs or derivatives thereof or the EVs or derivatives thereof of the present disclosure can be delivered in human body. In non-limiting examples, the disorder could be one or more of rheumatoid arthritis, multiple sclerosis, type I diabetes mellitus, idiopathic inflammatory myopathy, systemic lupus erythematosus (SLE), myasthenia gravis, Grave's disease, dermatomyositis, polymyositis, Crohn's disease, ulcerative colitis, gastritis, Hashimoto's thyroiditis, asthma, psoriasis, psoriatic arthritis, dermatitis, systemic scleroderma and sclerosis, inflammatory bowel disease (IBD), respiratory distress syndrome, meningitis, encephalitis, uveitis, glomerulonephritis, eczema, atherosclerosis, leukocyte adhesion deficiency, Raynaud's syndrome, Sjorgen's syndrome, Reiter's disease, Beheet's disease, immune complex nephritis, IgA nephropathy, IgM polyneuropathies, immune-mediated thrombocytopenias e.g., ITP), acute idiopathic thrombocytopenic purpura, chronic idiopathic thrombocytopenic purpura, hemolytic anemia, myasthenia gravis, lupus nephritis, atopic dermatitis, pemphigus vulgaris, opsoclonus-myoclonus syndrome, pure red cell aplasia, mixed cryoglobulinemia, ankylosing spondylitis, hepatitis C-associated cryoglobulinemic vasculitis, chronic focal encephalitis, bullous pemphigoid, hemophilia A, membranoproliferative glomerulonephritis, adult and juvenile dermatomyositis, adult polymyositis, chronic urticaria, primary biliary cirrhosis, neuromyelitis optica, Graves' dysthyroid disease, bullous pemphigoid, membranoproliferative glomerulonephritis, Churg-Strauss syndrome, juvenile onset diabetes, hemolytic anemia, atopic dermatitis, systemic sclerosis, Sjorgen's syndrome and glomerulonephritis, dermatomyositis, ANCA, aplastic anemia, autoimmune hemolytic anemia (AIHA), factor VIII deficiency, hemophilia A, autoimmune neutropenia, Castleman's syndrome, Goodpasture's syndrome, solid organ transplant rejection, graft versus host disease (GVHD), autoimmune hepatitis, lymphoid interstitial pneumonitis, HIV, bronchiolitis obliterans (non-transplant), Guillain-Barre Syndrome, large vessel vasculitis, giant cell (Takayasu's) arteritis, medium vessel vasculitis, Kawasaki's Disease, polyarteritis nodosa, Wegener's granulomatosis, microscopic polyangiitis (MPA), Omenn's syndrome, Alzheimer, chronic renal failure, acute infectious mononucleosis, HIV and herpes virus associated diseases.

### Methods and Compositions

The present disclosure also provides a method and compositions of treating a disease or condition with the PLC or EV population or a combination thereof of the present disclosure. In some embodiments, the PLCs and/or EVs or derivatives thereof can be administered to a patient in need thereof to augment or cure platelet-based deficiencies, as discussed in the forgoing. The method comprising administering to the patient in need there a therapeutic amount of PLCs and/or EVs or derivatives thereof or a combination thereof. In some embodiments, cytotoxic agents (e.g., a protein or a peptide, such as an antibody or a fragment thereof, a receptor or a portion thereof or a ligand or a fragment thereof, a drug or a prodrug) whether conjugated to a PLC and/or to EV via a linker (i.e., PLCs-L-C bioconjugates or EV-L-C) or directly conjugated to the PLC and/or EV or exogenously expressed in PLCs and/or EV or diffused into PLCs and/or EV (e., in PLC or EV granules) are administered in therapeutic amounts into a patient in need for treating a disease or condition with the cytotoxic agents. In these embodiments, the cytotoxic agents take advantage of the PLCs' or EVs' local or systemic or rolling, adhesion, and aggregate formation capabilities to travel (roll) from a first location, where the PLCs and/or EVs or derivatives thereof are administered, to a second location i.e., a diseased location, where the PLCs and/or EVs or their derivatives adhere to and aggregate to mitigate or eliminate a disease. As an example, the present disclosure provides a method for treating a patient having a neoplasm comprising administering to said patient a therapeutically effective amount of an the non-naturally occurring PLC and/or EV cell population or derivatives thereof or pharmaceutical composition described herein. The neoplasm is selected from, but not limited to, one or more of abdominal, bone, breast, digestive system, liver, pancreas, peritoneum, adrenal, parathyroid, pituitary, testicles, ovary, thymus, thyroid, eye, head and neck, central nervous system, peripheral nervous system, lymphatic system, pelvic, skin, soft tissue, spleen, thoracic region, and urogenital system. In some embodiments, the method comprises administering a second anti-cancer agent to the subject. In some embodiments, the second anti-cancer agent is a chemotherapeutic agent. The first and the second agent could be same or different depending upon the need of a patient. Thus, the first and/or the second agent could be selected from one or more of an anti-CD20 therapeutic, an anti-IL-6 receptor therapeutic, an anti-IL-12/23p40 therapeutic, an immunosuppressant, an anti-Interferon beta-1a therapeutic, glatiramer acetate, an anti-alpha4-integrin therapeutic, fingolimod, an anti-BLyS therapeutic, CTLA-Fc, or an anti-TNF therapeutic.

For use in a method of treating a disease or condition with a cytotoxic agent (e.g., protein, peptide antibody or a drug), which inhibits RNA polymerase, the present disclosure also provides PLCs and/or EVs or derivatives thereof in which the cytotoxic agent inhibits RNA polymerase and is prepared for administration with another therapeutic agent. The present disclosure also provides another therapeutic co-agent for use in a method of treating a disease or condition with a cytotoxic agent which inhibits RNA polymerase, wherein the other therapeutic co-agent is prepared for administration with the PLCs and/or EVs or derivatives thereof.

Typically, the PLCs and/or EVs or derivatives thereof are administered in a therapeutically effective dose. In view of the increased therapeutic efficacy, administration may occur less frequent as in treatment with conventional cell-based therapy or with bioconjugates and/or in a lower dose. Alternatively, in view of the increased tolerability, administration may occur more frequent as in treatment with conventional cell-based therapy or with bioconjugates and/or in a higher dose. Administration may be in a single dose or may e.g., occur every 3 to 4 hours, 1-4 times a day, 1-4 times a week, 1-4 times a month, possibly 1-7 times a week, or possibly administration occurs once every 3 or 4 weeks. As will be appreciated by the person skilled in the art, the dose of the PLCs and/or EVs or derivatives thereof, according to the present disclosure, may depend on many factors and optimal doses can be determined by the skilled person via routine experimentation.

The PLCs and/or EVs or derivatives thereof, are genetically engineered to produce a protein or polypeptide of interest, e.g., an Ipilimumab, secukinumab, trastuzumab antibody or a fragment thereof, or the PLC or EV bioconjugates (e.g., PLC or EV conjugated to a ligand or a receptor with or without a linker) may be used in vitro, ex vivo, or incorporated into pharmaceutical compositions and administered to individuals (e.g., human subjects) in vivo to treat, ameliorate, or prevent a disease or a disorder treatable by Ipilimumab, secukinumab, trastuzumab or with the PLCs or EV-bioconjugates of the present disclosure. A pharmaceutical composition will be formulated to be compatible with its intended route of administration (e.g., routes that are commonly followed during blood transfusion but performed with PLCs or derivatives thereof or EVs or derivatives thereof of the present disclosure or through oral compositions generally include an inert diluent or an edible carrier). Other nonlimiting examples of routes of administration include parenteral (e.g., intravenous or intravenous infusion), intradermal, subcutaneous, oral (e.g., inhalation), transdermal (topical), transmucosal, and rectal administration. The pharmaceutical compositions compatible with each intended route are well known in the art.

Treatment with the PLCs/EVs or Derivatives Thereof

The composition comprising PLCs and/or EVs or derivatives thereof will be formulated, dosed, and administered in a fashion consistent with good medical practice. Factors for consideration in this context include the particular disease or disorder being treated, the particular mammal being treated, the clinical condition of the individual patient, the cause of the disease or disorder, the site of delivery of the agent, the method of administration, the scheduling of administration, and other factors known to medical practitioners. The therapeutically effective amount of the PLCs and/or EVs or derivatives thereof to be administered will be governed by such considerations.

As a general proposition, the therapeutically effective amount of PLCs and/or EVs or derivatives thereof administered parenterally per dose will be in the range of about 0.01 to 1000 mg/kg of patient body weight per day, with the typical initial range of PLCs or derivatives thereof used being in the range of about, 0.03-300 mg/kg or 0.05-100 mg/kg, or alternatively 0.1-75 mg/kg or 0.5-50 mg/kg.

Suitable dosages for PLCs and/or EVs or derivatives thereof are, for example, in the range from about 20 mg/kg to about 1000 mg/kg. For example, one may administer to the patient one or more doses of substantially less than 375 mg/kg of the, PLCs and/or EVs or derivatives thereof e.g., where the dose is in the range from about 20 mg/kg to about 250 mg/kg, for example from about 50 m g/kg to about 200 mg/kg. For example, the initial dose may be in the range from about 0.1 mg/kg to about 100 mg/kg, or10 mg/kg to about 250 mg/kg (e.g., doses of 0.3-60 mg/kg over a 2-h infusion including 4 weekly doses of 15 or 30 mg/kg) and the subsequent dose may be in the range from about 1 mg/kg to about 10 mg/kg. Administration may be in a single dose or may e.g., occur every 3 to 4 hours, 1-4 times a day, 1-4 times a week; 1-4 times a month, possibly 1-7 times a week, or possibly administration occurs once every 3 or 4 weeks. As will be appreciated by the person skilled in the art, the dose of the PLCs and/or EVs or derivatives thereof, according to the present disclosure, may depend on many factors and optimal doses can be determined by the skilled person via routine experimentation.

These suggested amounts of PLCs and/or EVs or derivatives thereof are subject to a great deal of therapeutic discretion. The key factor in selecting an appropriate dose and scheduling is the result obtained, as indicated above. For example, relatively higher doses may be needed initially for the treatment of ongoing and acute diseases. To obtain the most efficacious results, depending on the disease or disorder, the PLCs and/or EVs or derivatives thereof is administered as close to the first sign, diagnosis, appearance, or occurrence of the disease or disorder as possible or during remissions of the disease or disorder.

In some embodiments, a diagnostic method or method of screening is provided for toxic agents such as autoimmune autoantibodies, antigens, viral or bacterial protein, or any other biological or chemical toxins, comprising: (a) obtaining a sample from a subject in which the presence of one or more of these agents is suspected; (b) admixing with the patient sample a composition comprising PLCs or EVs, platelet variants or derivatives thereof that exogenously or endogenously express one or more receptors/ligands/antigens for a counterpart ligand/receptor or an antigen to which agents such as autoimmune antibodies, or viral entry receptor proteins interact with or bind to; and (c) determining the presence or absence of the autoimmune antibody, or the bacterial or viral particles or viral peptides or viral nucleic acids in the patient's sample. Labelling techniques such as radiolabel, a fluorophore, a chromophore, an imaging agent or a metal ion may be employed to assist in such diagnosis. Labeling techniques are well known to one of skill in the art. In some embodiments, PLC-toxins or EV-toxins can be generated from the PLCs or derivatives thereof or thre EVs or the derivatives thereof. PLC/EV-toxins are made from a toxin attaching (for example, by genetic engineering or by bioconjugation or chemical conjugation) to PLC or PLC derivative target proteins or to EV or EV derivative target proteins present on a target cell (e.g., cancer cells, autoimmune antibodies or cells generating such antibodies, or viruses or bacteria or particles or proteins thereof) Here the PLC or EV portion of the molecule directs it to a specific antigenic determinant on a target cell; the molecule is then internalized, or is complexed and a cytotoxic reaction occurs.

The PLCs and/or EVs or derivatives thereof is administered by any suitable means, including parenteral, subcutaneous, intraperitoneal, intrapulmonary, and intranasal, and, if desired for local immunosuppressive treatment, intralesional administration. Parenteral infusions include intramuscular, intravenous, intraarterial, intraperitoneal, or subcutaneous administration. In addition, the PLCs or derivatives thereof may suitably be administered by pulse infusion, e.g., with declining doses of the PLCs or derivatives thereof. In some embodiments the dosing is given by injections, for example via intravenous or subcutaneous injections, depending in part on whether the administration is brief or chronic.

One may administer other compounds, such as cytotoxic agents, immunosuppressive agents and/or cytokines with the PLCs and/or EVs or derivatives thereof herein. The combined administration includes co administration, using separate formulations or a single pharmaceutical formulation, and consecutive administration in either order, wherein preferably there is a time period while both (and all) active agents simultaneously exert their biological activities.

Pharmaceutical composition comprising the PLCs and/or EVs or derivatives thereof of the present disclosure may be combined with a pharmaceutically acceptable carrier. Such a composition may contain, in addition to PLCs and/or EVs or derivatives thereof, carriers, various diluents, fillers, salts, buffers, stabilizers, solubilizers, and other materials well known in the art. The characteristics of the carrier will depend on the route of administration. The pharmaceutical compositions for use in the disclosed methods may also contain additional therapeutic agents for treatment of the particular targeted disorder. For example, a pharmaceutical composition may also include other agents as disclosed herein. Such additional factors and/or agents may be included in the pharmaceutical composition to produce advantages of the therapeutic approaches disclosed herein, i.e., provide improved drug efficacy with reduced systemic toxicity.

Therapeutic formulations of the PLCs and/or EVs or derivatives thereof used in accordance with the present disclosure are prepared for storage by mixing PLCs and/or EVs or derivatives thereof having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS' or polyethylene glycol(PEG), for example, a PEG chain having a molecular weight between 1,000-15,000 daltons, or between 2,000 and 10,000 daltons, or between 2,000 and 5,000 daltons. Other hydrophilic polymers which may be suitable include polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses, such as hydroxymethylcellulose or hydroxyethylcellulose.

Lyophilized formulations adapted for subcutaneous administration are also contemplated by the disclosure. Such lyophilized formulations may be reconstituted with a suitable diluent to an optimal concentration and the reconstituted formulation may be administered subcutaneously to the mammal to be treated herein.

The formulation herein may also contain more than one active compound as necessary for the particular indication being treated, preferably those with complementary activities that do not adversely affect each other. For example, it may be desirable to further provide a cytotoxic agent, cytokine or immunosuppressive agent. The effective amount of such other agents depends on the amount of PLCs and/or EVs or derivatives thereof present in the formulation, the type of disease or disorder or treatment, and other factors discussed above. These are generally used in the same dosages and with administration routes as used hereinbefore or about from 1 to 99% of the heretofore employed dosages.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semi permeable matrices of solid hydrophobic polymers containing the PLCs or derivatives thereof, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma ethyl-L-glutamate, non-degradable ethylene-vinyl acetate, degradable lactic acid-glycolic acid copolymers such as the LUPRON DEPOT′ (injectable microspheres composed of lactic acid-glycolic acid copolymer and leuprolide acetate), and poly-D-(−)-3-hydroxybutyricacid.

The formulations to be used for in vivo administration must be sterile. This is readily accomplished by filtration through sterile filtration membranes and other techniques known to one of skill in the art.

In various embodiments, the pharmaceutical composition may be formulated to suit any desired mode of administration. For example, the pharmaceutical composition can take the form of solutions, suspensions, emulsion, drops, tablets, pills, pellets, capsules, capsules containing liquids, gelatin capsules, powders, sustained-release formulations, suppositories, emulsions, aerosols, sprays, suspensions, lyophilized powder, frozen suspension, desiccated powder, or any other form suitable for use. General considerations in the formulation and manufacture of pharmaceutical agents may be found, for example, in Remington's Pharmaceutical Sciences, 19th ed., Mack Publishing Co., Easton, Pa., 1995; incorporated herein by reference.

The present pharmaceutical composition may be administered in any dose appropriate to achieve a desired outcome. In some embodiments, the desired outcome is the induction of a long-lasting adaptive immune response against a pathogen, such as the source of a non-ferritin polypeptide present in an antigenic ferritin polypeptide present in the composition. In some embodiments, the desired outcome is a reduction in the intensity, severity, frequency, and/or delay of onset of one or more symptoms of infection. In some embodiments, the desired outcome is the inhibition or prevention of infection. The dose required will vary from subject to subject depending on the species, age, weight, and general condition of the subject, the severity of the infection being prevented or treated, the particular composition being used, and its mode of administration.

In some embodiments, pharmaceutical compositions in accordance with the present disclosure are administered in single or multiple doses. In some embodiments, the pharmaceutical compositions are administered in multiple doses administered on different days.

In various embodiments, the pharmaceutical composition is co-administered with one or more additional therapeutic agents. Co-administration does not require the therapeutic agents to be administered simultaneously, if the timing of their administration is such that the pharmacological activities of the additional therapeutic agent and the active ingredient(s) in the pharmaceutical composition overlap in time, thereby exerting a combined therapeutic effect. In general, each agent will be administered at a dose and on a time, schedule determined for that agent.

In some embodiments, the administration of the composition comprising the PLCs and/or EVs or their derivatives, according to the present disclosure, is at a dose that is lower than the toxic dose (TD₅₀) of the donor platelets or bioconjugates thereof but not comprising the PLCs and/or EVs or their derivatives or bioconjugates thereof of the present disclosure. For example, the dose is at most 99-90%, 89-80%, 79-70% 69-60%, 59-50%, 49-40%, 39-30%, 29-20%, 19-10%, 9-1% or 0.9-0.01% lower than the toxic dose (TD₅₀) of the donor platelets or bioconjugates thereof but not comprising the PLCs and/or EVs or their derivatives or bioconjugates thereof of the present disclosure. Alternatively, the administration of the donor platelets or bioconjugates thereof according to the present disclosure is at a dose that is higher than the TD₅₀ of the donor platelets or bioconjugates thereof but not comprising PLCs and/or EVs or their derivatives or bioconjugates thereof. For example, the dose is at most 0.9 to 0.01%, 9-1%, 19-10%, 29-20%, 39-30%, 49-40%, 59-50%, 69-60%, 79-70%, 89-80%, 99-90% higher than the TD₅₀ of the donor platelet or bioconjugate thereof but not comprising the PLCs and/or EVs or their derivatives or bioconjugates thereof of the present disclosure.

In some embodiments, the administration of the PLCs and/or EVs or derivatives thereof, according to the present disclosure, is at a dose that is lower than the ED₅₀ of the donor platelets or bioconjugate thereof but not comprising the PLCs and/or EVs or their derivatives or bioconjugates thereof of the present disclosure. For example, 99-90%, 89-80%, 79-70% 69-60%, 59-50%, 49-40%, 39-30%, 29-20%, 19-10%, 9-1% lower than the effective dose (ED₅₀) of the donor platelet or bioconjugate thereof but not comprising the PLCs and/or EVs or their derivatives or bioconjugates thereof of the present disclosure. Alternatively, the administration of the PLCs and/or EVs or their derivatives or bioconjugates thereof, according to the present disclosure, is at a dose that is higher than the ED₅₀ of the same cell-based therapy or bioconjugate but not comprising the PLCs and/or EVs or their derivatives or bioconjugates thereof of the present disclosure. For example, the dose is at most 0.9 to 0.01%, 9-1%, 19-10%, 29-20%, 39-30%, 49-40%, 59-50%, 69-60%, 79-70%, 89-80%, 99-90% higher than the ED₅₀ of the donor platelet or bioconjugate thereof but not comprising the PLCs and/or EVs or their derivatives or bioconjugates thereof of the present disclosure.

Herein, the term “therapeutic index” (TI) has the conventional meaning well known to a person skilled in the art and refers to the ratio of the dose of drug that is toxic (i.e., causes adverse effects at an incidence or severity not compatible with the targeted indication) for 50% of the population (TD₅₀) divided by the dose that leads to the desired pharmacological effect in 50% of the population (effective dose or ED₅₀). Hence, TI=TD₅₀/ED₅₀. The therapeutic index may be determined by clinical trials or for example by plasma exposure tests. See also Muller, et al. Nature Reviews Drug Discovery 2012, 11, 751-761. At an early development stage, the clinical TI of a drug candidate is often not yet known. However, understanding the preliminary TI of a drug candidate is of utmost importance as early as possible since TI is an important indicator of the probability of the successful development of a drug. Recognizing drug candidates with potentially suboptimal TI at earliest possible stage helps to initiate mitigation or potentially re-deploy resources. At this early stage, TI is typically defined as the quantitative ratio between safety (maximum tolerated dose in mouse or rat) and efficacy (minimal effective dose in a mouse xenograft).

Herein, the term “therapeutic efficacy” denotes the capacity of a substance to achieve a certain therapeutic effect, e.g., reduction in tumor volume. Therapeutic effects can be measured determining the extent in which a substance can achieve the desired effect, typically in comparison with another substance under the same circumstances. A suitable measure for the therapeutic efficacy is the ED₅₀ value, which may for example be determined during clinical trials or by plasma exposure tests. In case of preclinical therapeutic efficacy determination, the therapeutic effect of a bioconjugate (e.g., PLCs and/or EVs or derivatives thereof), can be validated by patient-derived tumor xenografts in mice in which case the efficacy refers to the ability of the PLCs or derivative thereof to provide a beneficial effect. Alternatively, the tolerability of said PLCs and/or EVs or derivatives thereof in a rodent safety study can also be a measure of the therapeutic effect.

Herein, the term “tolerability” refers to the maximum dose of a specific substance that does not cause adverse effects at an incidence or severity not compatible with the targeted indication. A suitable measure for the tolerability for a specific substance is the TD₅₀ value, which may for example be determined during clinical trials or by other tests known to one of skill in the art.

Further details of the disclosure are illustrated by the following non-limiting Examples. These examples are put forth so as to provide those of ordinary skill in the art with a complete disclosure and description of how to make and use the compositions and methods of the disclosure, and are not intended to limit the scope of what the inventors regard as their disclosure.

### EXAMPLES

### Example 1: Structural Characterization of Platelet Like Cells (PLCs)

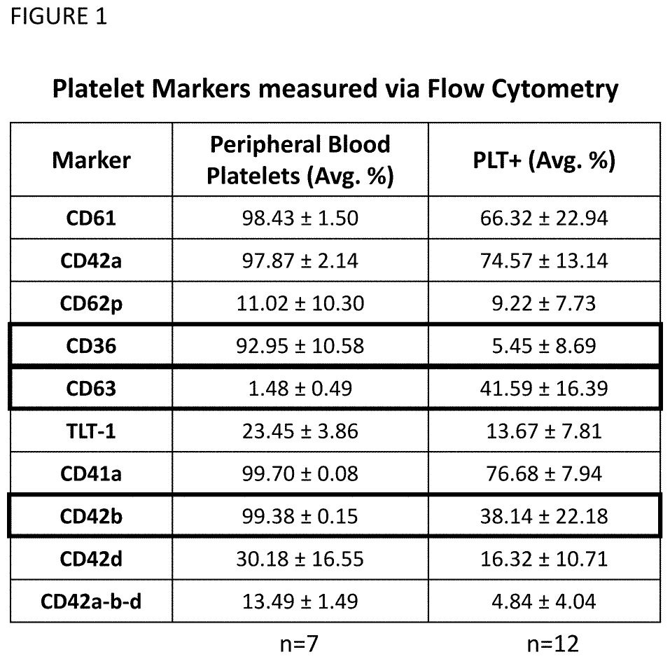

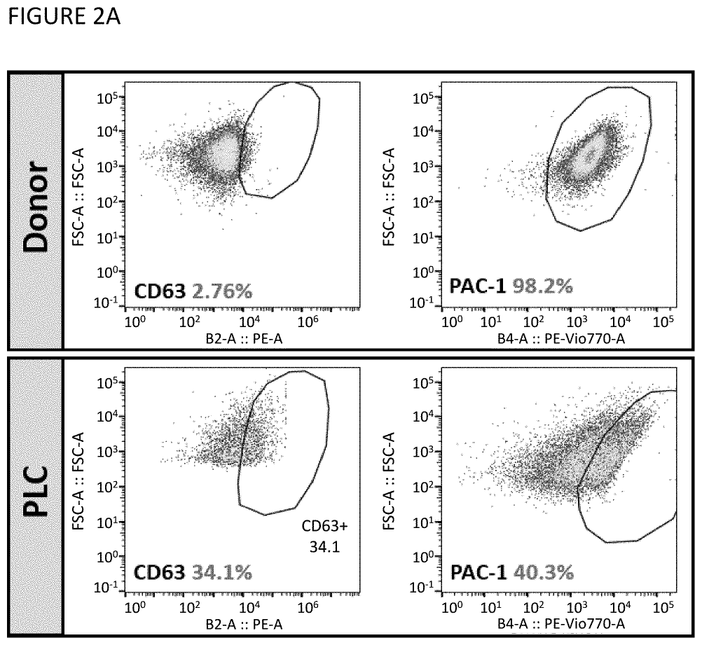

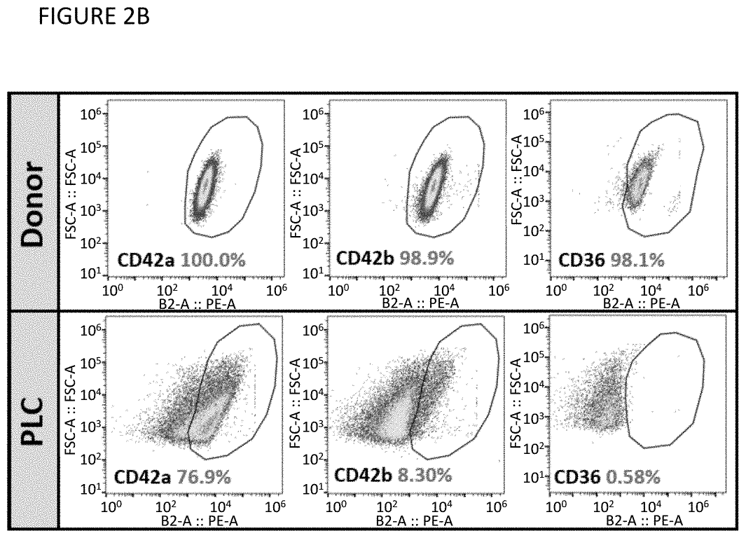

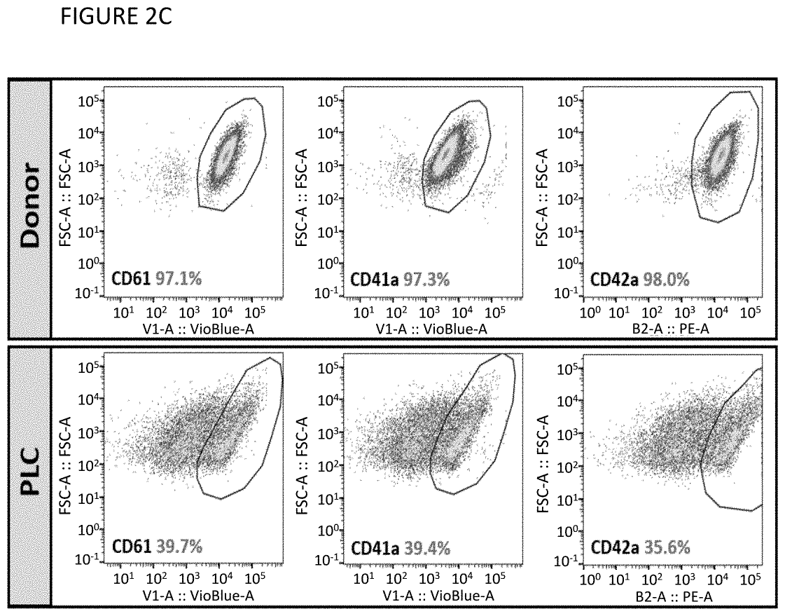

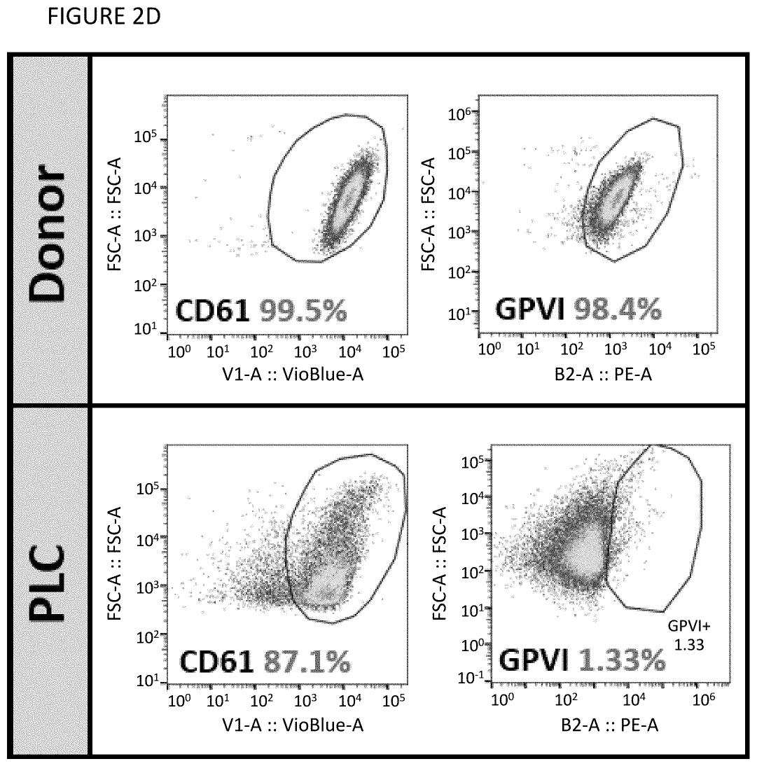

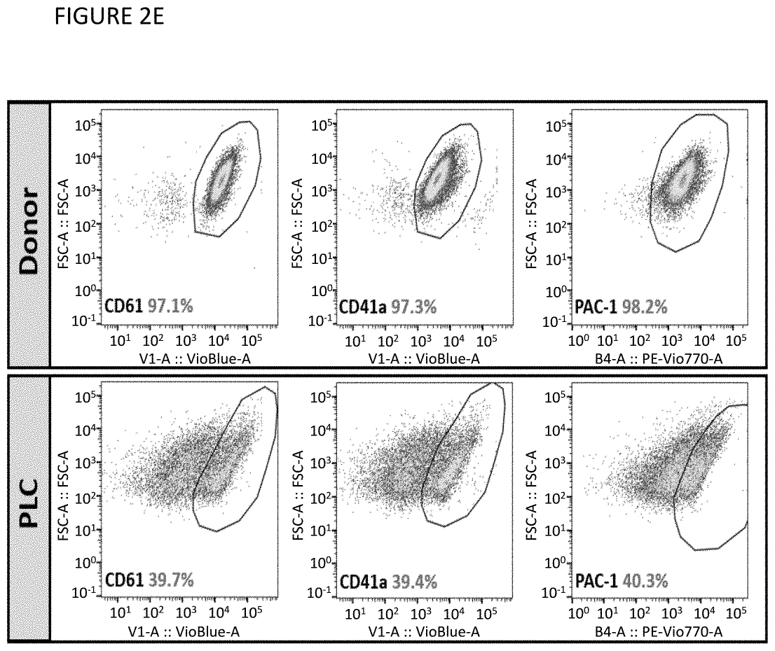

FIGS. 1 and 2A-2E show the structural makeup of the PLCs based on distributions of receptors or ligands on PLCs cell surface or in their transmembrane domain (FIGS. 1 & 2A-E). Structural makeup of PLCs was compared to donor platelets via flow cytometry. FIGS. 2A-E demonstrate that the PLCs structurally differ in the distribution of CD63 and PAC-1 (FIG. 2A) CD36 CD42b and CD42a (FIG. 2B); CD61 CD41a and CD42a (FIG. 2C); CD61 and GPVI (FIG. 2D) and CD61 CD41a and PAC-1 (FIG. 2E).

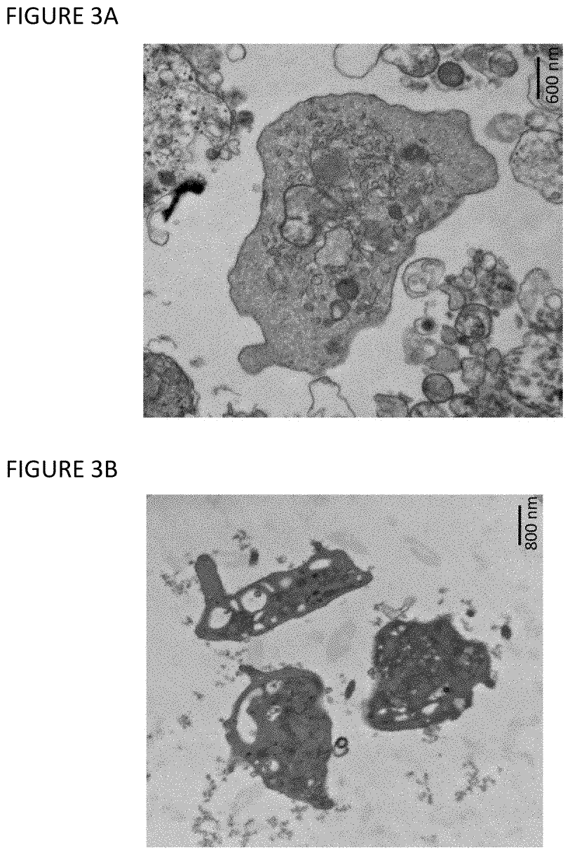

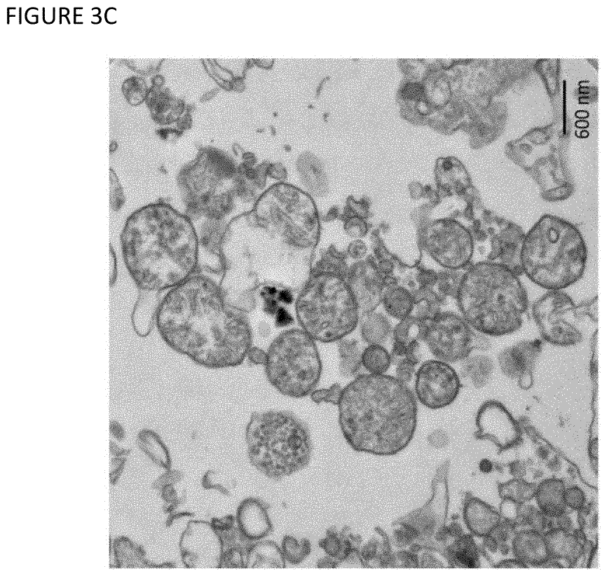

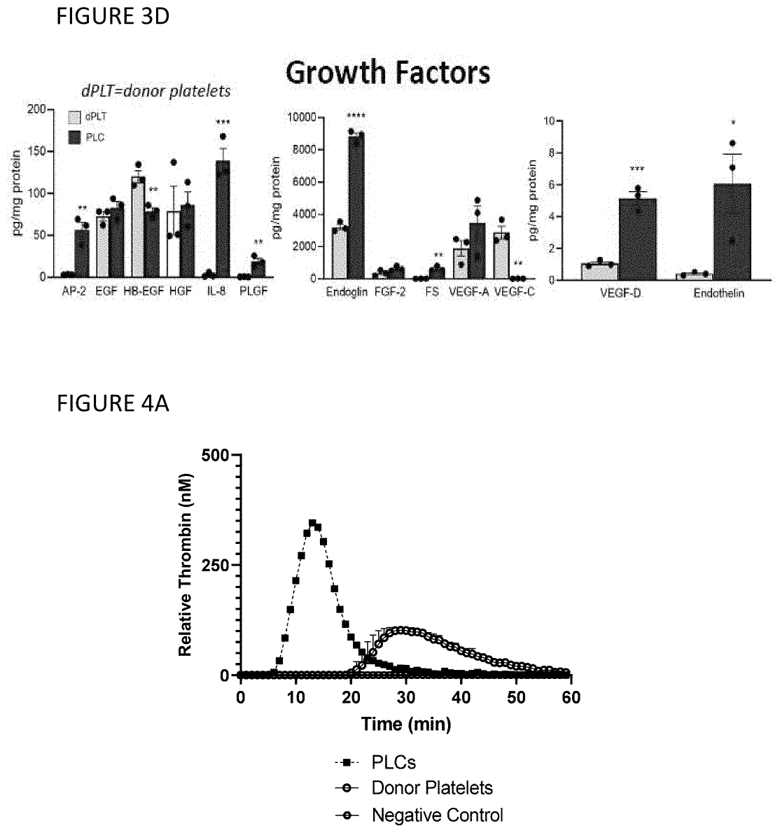

PLCs have a size distribution averaging between 65 nm to 10 μm (FIG. 3A), i.e., relatively larger than donor platelets, which are 2-3 μm (FIG. 3B) in greatest diameter (FIGS. 3A and 3B). In addition to the PLCs, the bioreactor generated platelets also produce microsomes and exosomes (FIG. 3C) as admixtures with the PLCs. FIG. 3D illustrates that PLCs express several growth factors, which are in greater quantity or are mostly in comparable quantity to that found in the donor platelets (dPLT). In some instances, concentration of certain growth or angiogenic factors in the PLCs may be less than that is found in the PLCs (e.g., PDGF-BB), which can advantageously be manipulated for therapeutic purposes depending on a patient's need.

### Example 2: Functional Characterization of PLCs

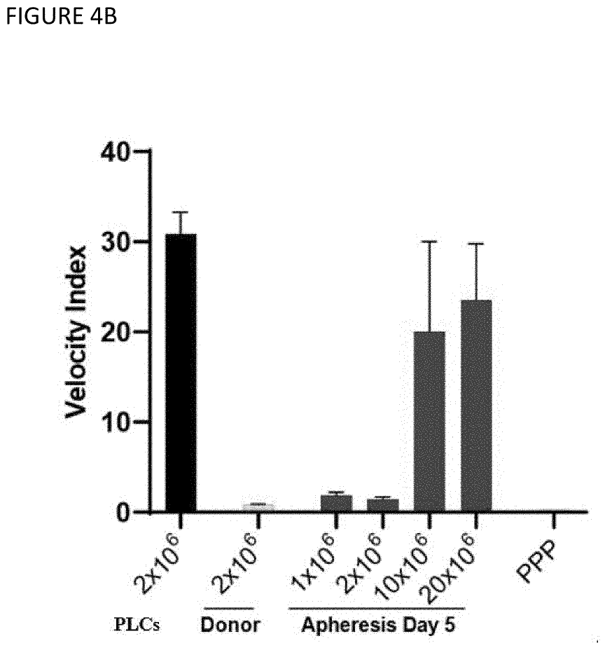

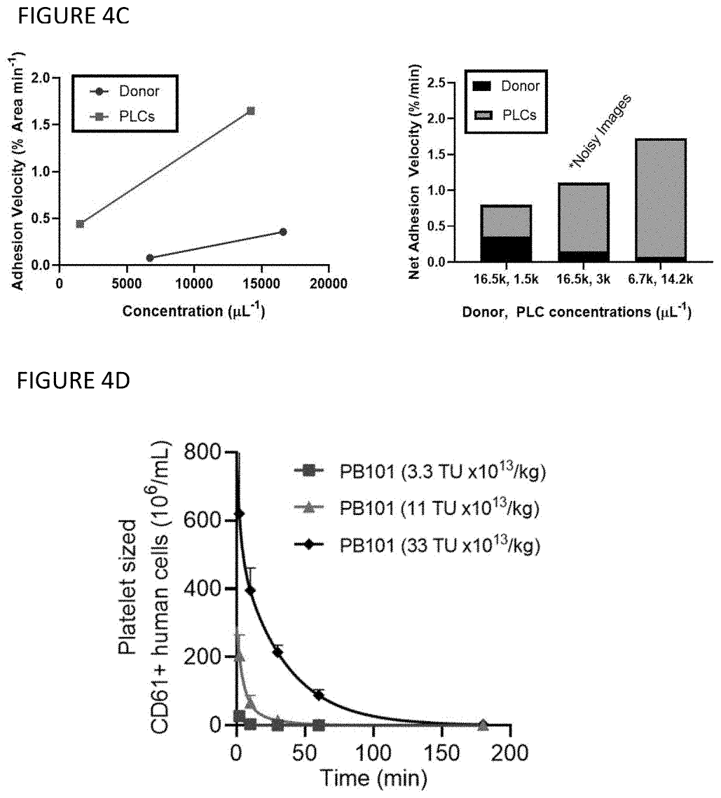

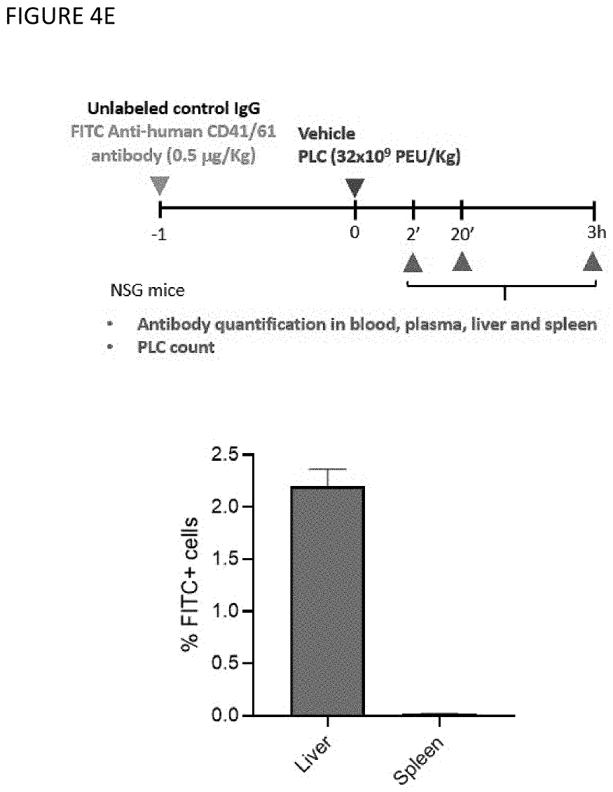

FIGS. 4A-4E are functional characterizations which distinguish PLCs from the donor platelets. PLCs generate a peak thrombin in greater abundance than donor platelets in plasma and do so in a shorter timeframe as indicated by the velocity index after being exposed to recombinant human tissue factor. To establish this, experiments were performed as follows. The Technothrombin Thrombin Generation Assay Kit (Diapharma #5006010) was used to evaluate the thrombin generation potential of PLCs. The thrombin generation assay measures the formation of thrombin and monitors the kinetics in real-time over the course of 60 minutes. The kinetic traces shown in FIG. 4A outlines the time for thrombin generation to initiate, time to peak production of thrombin, peak thrombin generation, and total thrombin generation (AUC). In this study, PLCs was dosed at 2×10\hat{ }6 CD61+ cells based on flow cytometry to matched donor platelet dose of 2×10\hat{ }6 cells. The assay indicates that PLCs with tissue factor generated a thrombin response that was greater than washed donor platelets (FIG. 4A). FIG. 4B demonstrates the velocity index which represents effective rate of thrombin generation between the lag time and time to peak thrombin generation. The velocity rate of PLCs exceeds fresh washed donor platelets and platelets stored within an apheresis for five days. The velocity index of platelet pore plasma was utilized as the background control.

FIG. 4C is another functional characterization which distinguishes PLCs from donor platelets. PLCs are more adhesive to collagen than the donor platelets. In order to demonstrate this, PLCs were added to the reconstituted blood (volume depending on PLC count, usually less than 1 and the solution was pipetted into the well. The sample was perfused over the collagen surface at 9.7 μL·min⁻¹, corresponding to a surface shear rate of 100 s⁻¹ for 5 minutes. Images were captured every 5 seconds with 470 nm and 640 nm excitation respectively for the DiOC₆ and Cell Tracker Deep Red stains, with a 20× objective on an inverted microscope (Leica Thunder). Images were analyzed using ImageJ to determine the surface coverage of both donor platelets and PLC at each time frame. The adhesion velocity was determined as the slope of the best fit line of any two (2) minute period using a custom Python routine. Values were plotted using Prism. For the figures, the brightness and contrast were adjusted for clarity.

FIG. 4D shows rapid clearance of the PLCs. Immunocompromised NOD scid gamma (NSG) mice (Jackson laboratory stock #005557) were dosed i.v. with PLC (3, 11, 33 10¹³/kg). Mouse blood was collected in EDTA tubes by tail vein transection 2, 20, 30 minutes, 1 hour and 3 hours following injection of PLC. PLC were counted by flow cytometry: Mouse blood was pre-diluted (1:30) in PBS. 5 μl of diluted blood was transferred into 45 μl of an antibody master mix containing PE-conjugated anti-mouse CD61 (1:100) and VB-conjugated anti-human CD61 (1:50) or isotypic control antibodies. Samples were incubated 20 minutes in the dark. 200 μl of PBS was added, and samples were analyzed using a MacsQuant flow cytometer.

Gating strategy: platelet-sized particles were analyzed for fluorescence. Events that were negative for PE-conjugated anti-mouse CD61 and positive for VB-conjugated anti-human CD61 were counted as PLC. The count/ml was calculated based on the volume acquired by the flow cytometer and multiplied by the dilution factor. As shown in FIG. 4D, PLCs have a relatively short circulation time in mice.

FIG. 4E demonstrates that the PLCs are liver bound, which may lead to clearance of toxic molecules (e.g., autoantibodies, bacterial or viral-induced toxins or chemical toxins, and other harmful molecules). In an exemplary study, to determine if PLCs can clear anti-CD41 antibody (96-2C1) and anti CD41/61 antibody (PAB-1) in vivo in liver, NSG immunodeficient mice were dosed with a fluorescently labeled mouse anti-human-CD41 (96-2C1) or CD41/61 FITC antibody (PAB-1) at 0.5 mg/kg. These antibodies do not recognize or cause clearance of mouse platelets. The fluorescence was quantified in homogenized livers and spleens. Results in FIG. 4E demonstrate that PLC-dependent anti-CD41 antibody (96-2C1) and anti CD41/61 antibody (PAB1) clearance occurs in the liver.

### Example 3: Covalent Conjugation of Drug Moiety to the PLCs

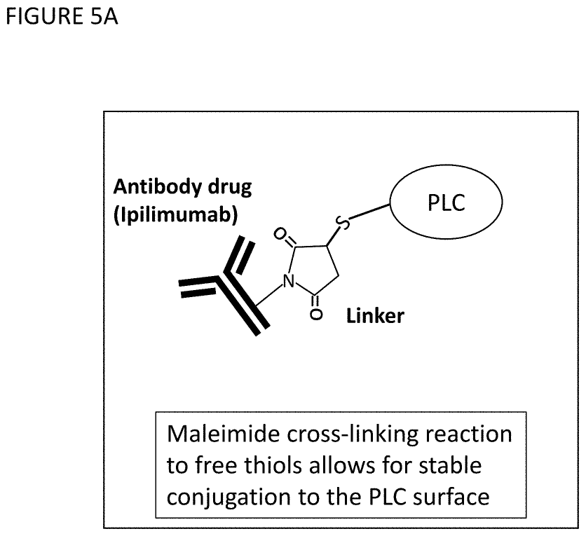

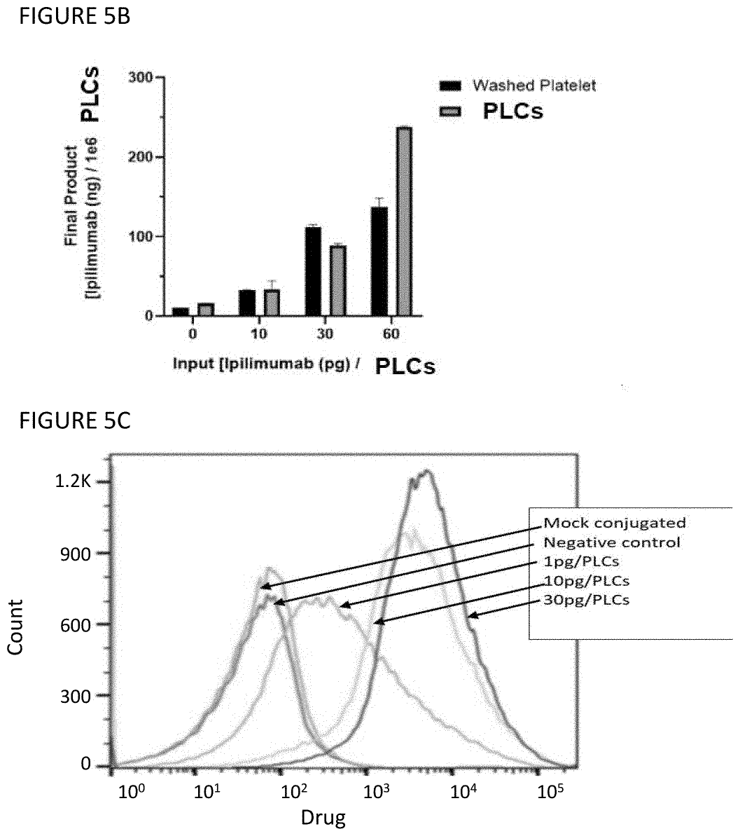

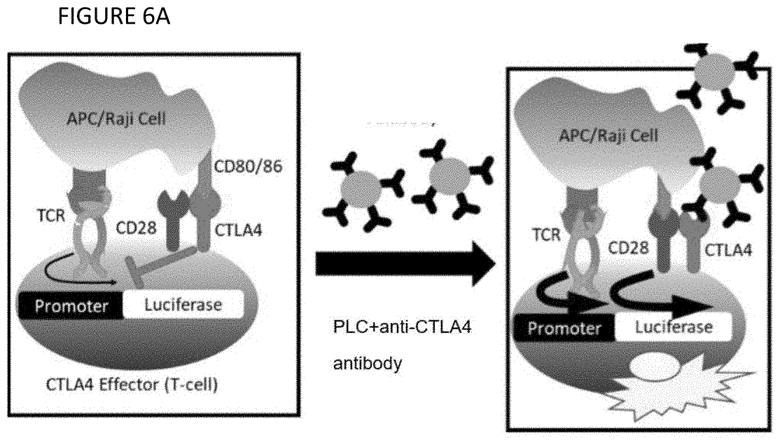

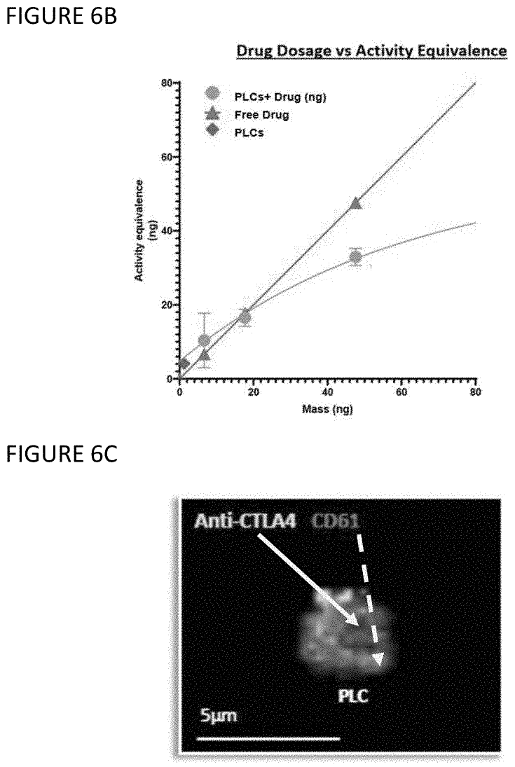

The ability to conjugate recombinant drug biologics to the cellular membrane of PLCs is demonstrated herein. PLCs were treated with Traut's Reagent at 0.4 mg/ml with 1e6 cells in 500 ul of buffer at 37° C. for 1 hour to convert primary amines to sulfhydryls. Concomitantly, SMCC was incubated with Ipilimumab for 2 hours at 4° C. (FIG. 5A). The SMCC linked Ipilimumab was reacted with the traut treated PLCs for 1 hour at 37° C. A secondary antibody against human IgG and conjugated to alexafluor 647 was used to detect the conjugated Ipilimumab (FIG. 5A). For drug treated sample, all observable PLCs had detectable Ipilimumab (FIGS. 5B-5C). This data demonstrates covalent conjugation of recombinant protein biologic drugs to PLCs. In a likewise manner, FIGS. 6A and 6B illustrate that in a functional assay, PLCs conjugated to anti-CTLA4 antibody regulate immune checkpoint inhibition. FIG. 6C is an immunostaining showing a bioconjugate comprising anti-CTLA4 mAb that was chemically conjugated to the surface of the PLC. Broken arrow on the surface of PLCs in FIG. 6C shows conjugation of CD61 to CTLA4 in the PLCs (shown by a regular arrow).

PLC conjugates can be administered to a patient in need of a treatment of a disease or disorder (e.g., melanoma, non-small cell lung carcinoma (NSCLC), small cell lung cancer (SCLC), bladder cancer and metastatic hormone-refractory prostate cancer), the effect of which can be mitigated by a cytotoxic agent attached to the PLCs. For example, a patient suffering from CTLA4-mediated cancer can be dosed with PLC-ipilimumab bioconjugate in an amount effective to stimulate the immune system by targeting CTLA4, a protein receptor that downregulates the immune system. Treatment regiments can be determined based on currently FDA approved regiments or a regimen deemed fit by a provider treating such patients.

### Example 4: Small Molecule Loading of PLCs by Passive Diffusion

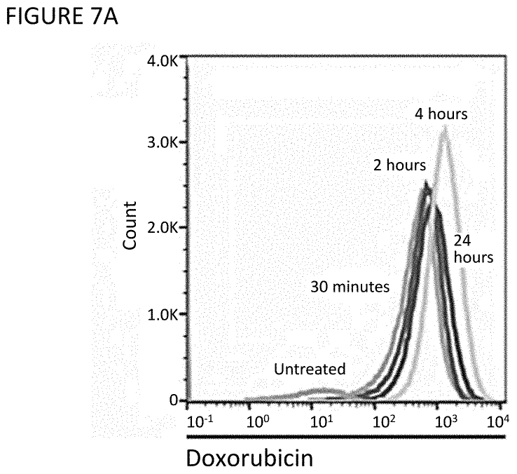

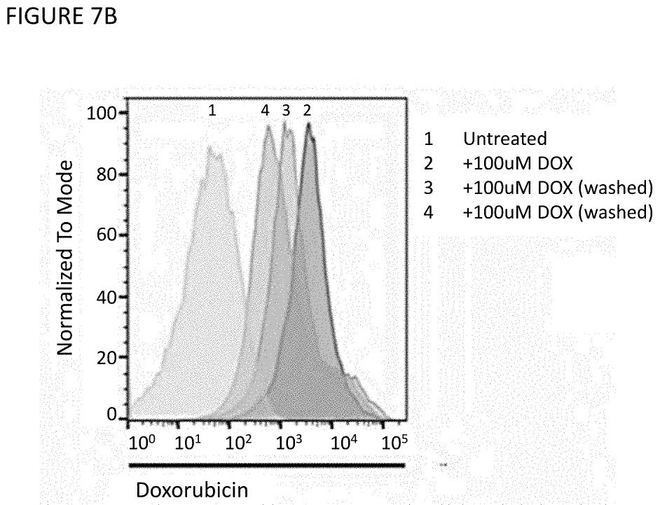

To demonstrate small molecule loading and retention in PLCs, PLCs were co-incubated with the DNA intercalating chemotherapeutic, Doxorubicin hydrochloride (Sigma #D1515). As anucleate PLCs do not contain genomic material that would typically sequester this drug within the PLCs because of co-incubation with a PLC preparation and entry into the cell by passive diffusion. 100 μM of Doxorubicin was used with a preparation of 1e7 PLCs in 1 ml of buffer and incubated at ambient temperature for 30, 120, 240, and 1440 minutes under constant agitation on an orbital shaker in a dialysis cassette (Thermofisher #88400). Doxorubicin has intrinsic fluorescent properties that can be detected by flow cytometry (Ex 427 nm/Em 585 nm), and it was found that multiple wash steps could be performed on the PLC preparation and the drug cargo would still be retained after loss of non-specifically bound molecules (FIG. 7A). A kinetic study was employed to understand the minimum and maximum amount of time necessary for doxorubicin encapsulation in the washed PLCs. It was observed that 30 minutes was sufficient to see detectable doxorubicin expression in the PLCs imbibed with Doxorubicin, with the signal retained after 1440 minutes (FIG. 7B). This data suggests that small-molecule drugs can be efficiently captured in PLCs.

### Example 5: Extracellular Vehicles (EVs), their Isolation and Characterization

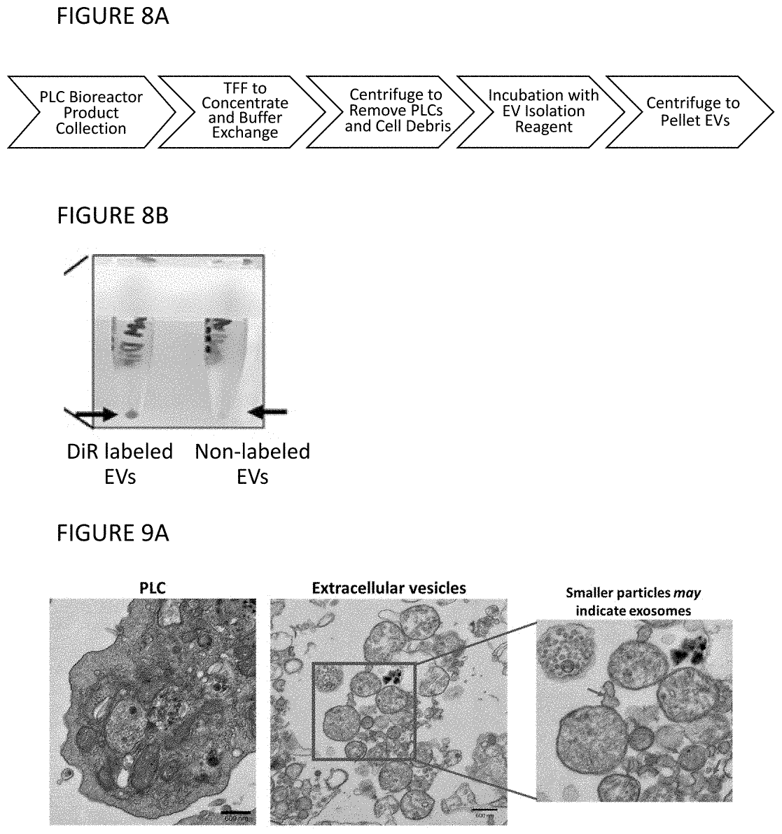

With respect to FIGS. 8A and 8B, a schematic illustration for the isolation of bioreactor-derived extracellular vesicles is shown.

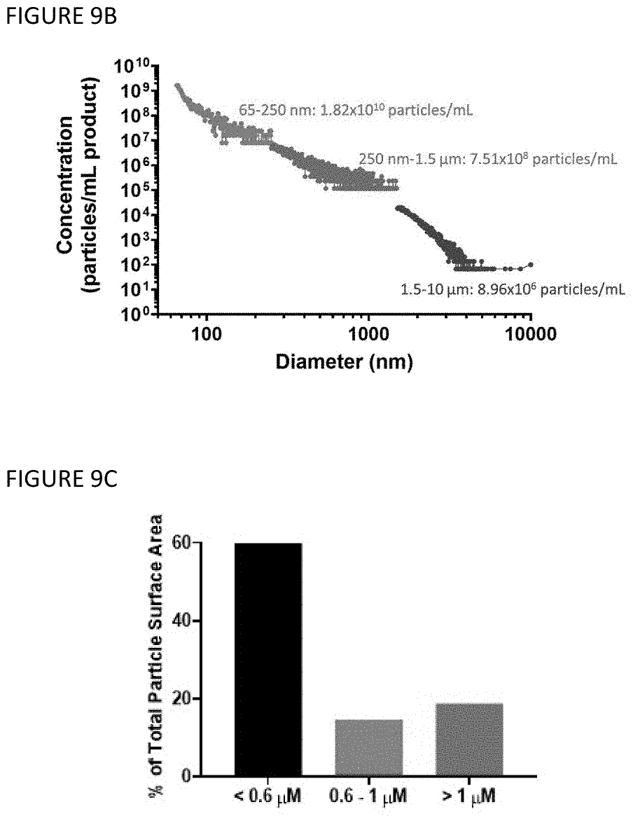

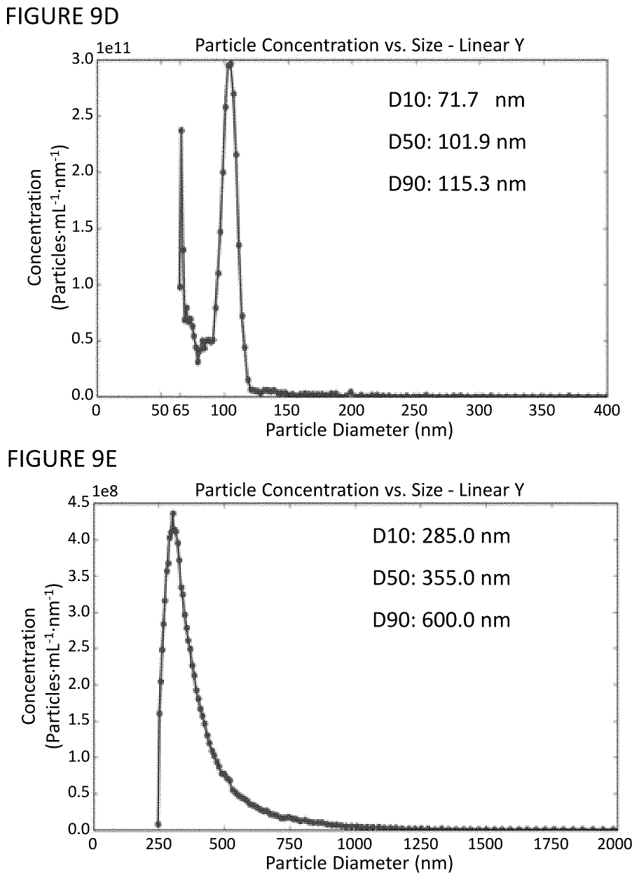

With respect to FIG. 9A, electron microscopy confirms membrane-bound structures are present in the EVs and are capable of carrying cargos. With respect to FIG. 9B, the bioreactor product contains a range of particle sizes ranging between from about 65 nm to about 10 μm. With respect to FIG. 9C, smaller particles represent much of the PLC surface area. With respect to FIG. 9D, the average diameter of isolated exosomes is approximately about 102 nm. With respect to FIG. 9E, the average diameter of isolated microparticles is approximately about 355 nm.

### Example 6: Characterization of Bioreactor-Derived EVs, Their Uptake into Cells

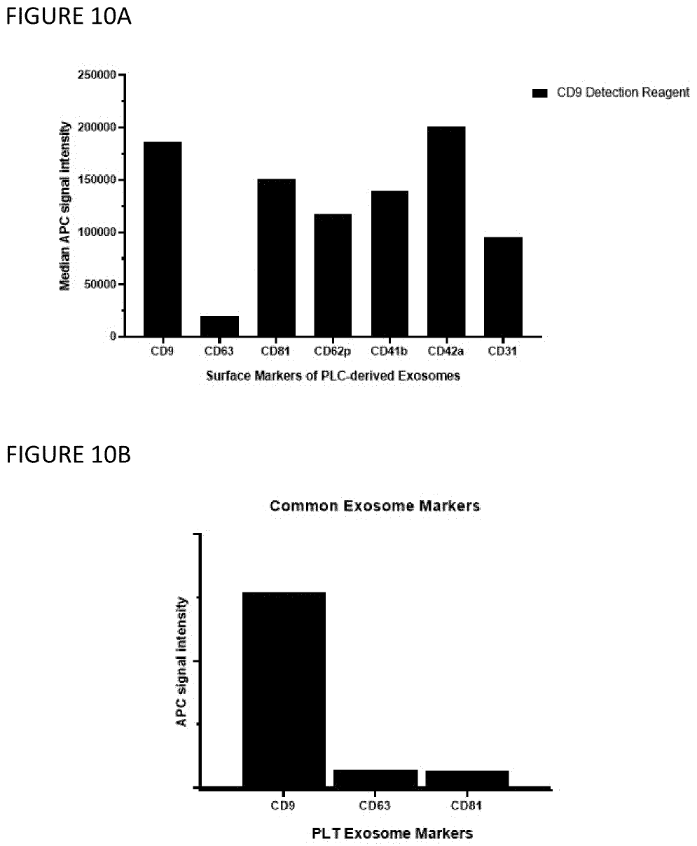

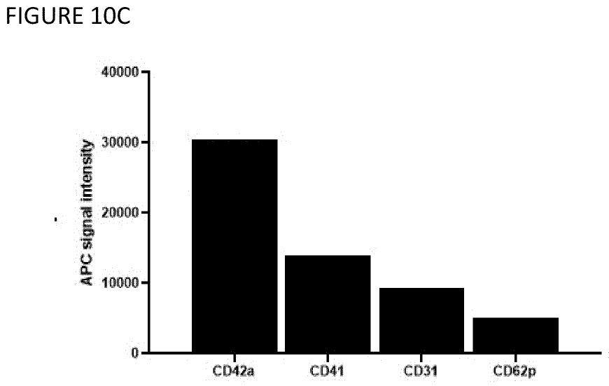

With respect to FIGS. 10A-10C, the bioreactor-derived EVs were characterized with surface markers using MACSPlex Assay. The bioreactor-derived EVs are positive of common exosome markers (CD9 CD63 and CD81) while CD9 has the highest expression; The bioreactor-derived EVs are also positive of platelet-related markers (CD62p, CD41b, CD42a and CD31).

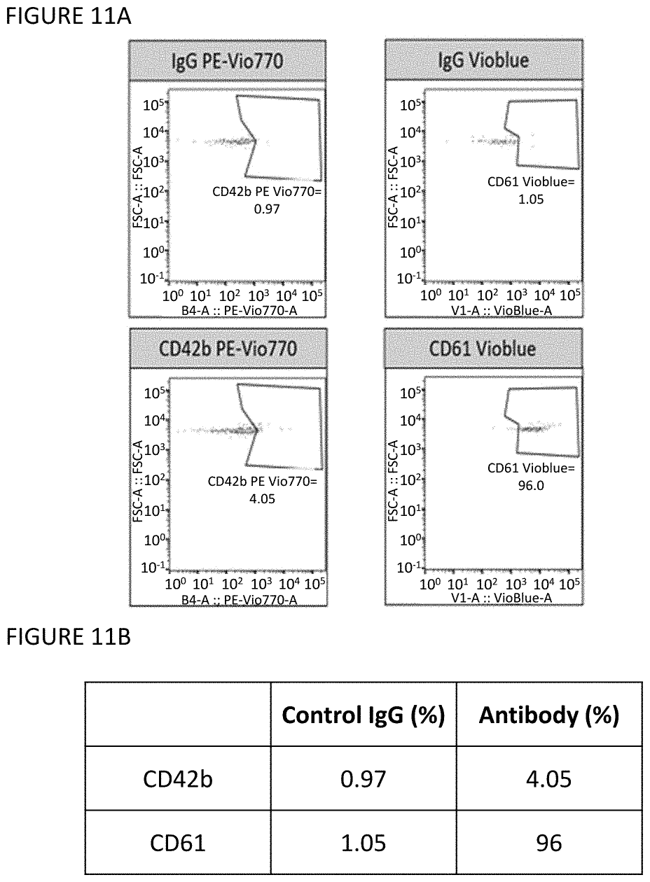

With respect to FIGS. 11A-11B, the bioreactor-derived EVs were further characterized with platelet-related surface markers using flow cytometry. They have slightly increased expression of CD42b (left panel), and significantly increased expression of CD61 compared to control IgG (right panel), summarized in FIG. 11B.

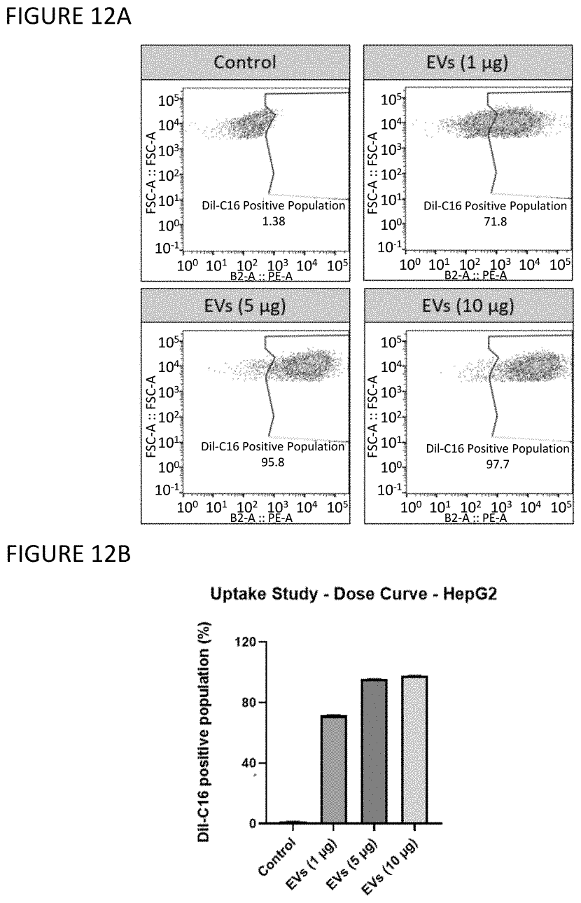

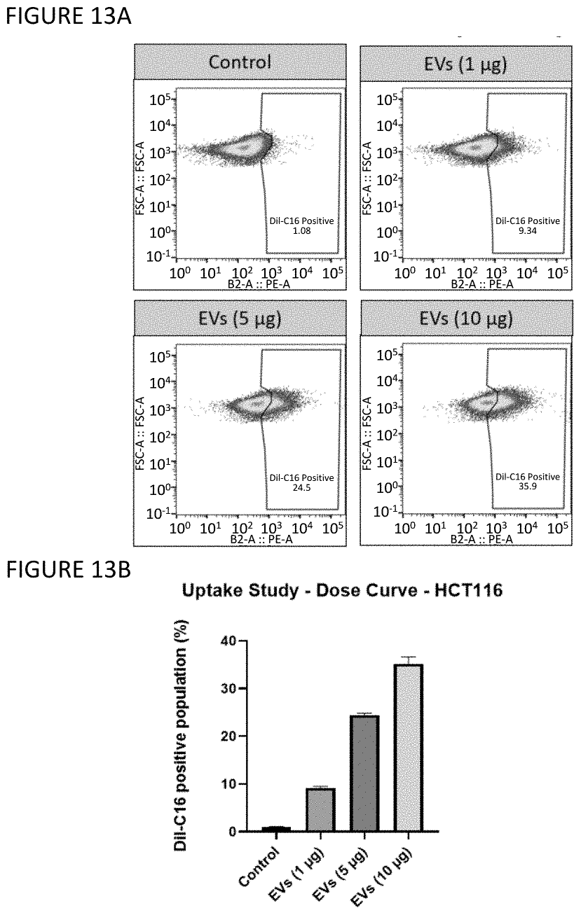

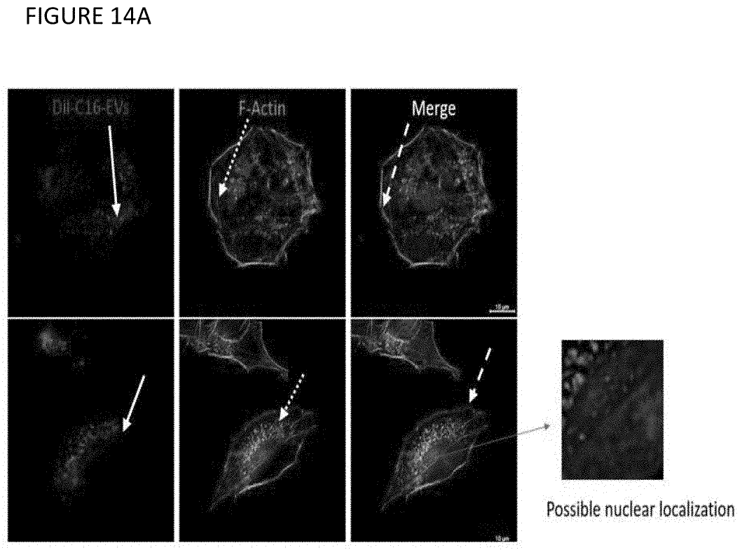

With respect to FIGS. 12A-12B and 13A-13B, the D6+4 pre-MLC were labeled by DiR dye at the concentration of 3 μM and incubated for 72 hours. The total EV production was characterized by nCS1 particle counter and the expression of common exosome markers and platelet-related markers was check using MACSPlex assay (FIG. 12A). In FIG. 13A, the bioreactor-derived EVs were labeled by DiI-C16 and DiR dyes at the concentration of 3 μM; Due to the small size of exosomes, they were captured by CD9 dynabeads (2.7 μm) to visualize the exosomes by flow cytometry. The results confirmed that the EVs were successfully labeled by DiI-C16 and DiR.

FIG. 12B show results of uptake studies, HepG2 cells (human hepatocellular carcinoma cell line) were co-incubated with DiI-C16 labeled EVs (1, 5 and 10 μg) for 3 hours, followed by flow cytometry to check the DiI-C16 intensity. Nearly 100% of HepG2 cells had detectable EVs at 5 μg, suggesting the efficient uptake of bioreactor derived EVs by HepG2 cells. Therefore, the bioreactor-derived EVs have the potential to serve as advanced drug delivery platform to treat a variety of cancers.

FIG. 13B, for uptake studies, HCT116 cells (human colon carcinoma cell line) were co-incubated with DiI-C16 labeled EVs (1, 5 and 10 μg) for 3 hours, followed by flow cytometry to check the DiI-C16 intensity. The HCT116 uptake bioreactor-derived EVs in a dose-dependent manner. The uptake of bioreactor-derived EVs by HCT116 cells is comparable to HepG2 cells, albeit with slightly less efficiency at the same dose, however, the amount of bioreactor-derived EVs can be further increased to achieve higher uptake due to the non-toxic and non-immunogenic properties of EVs.

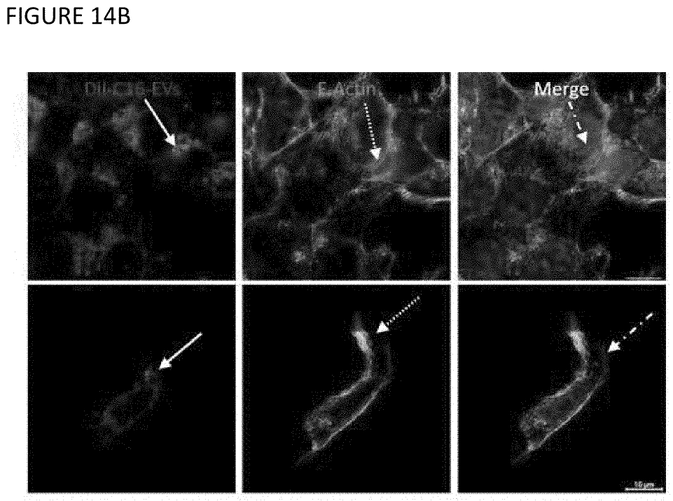

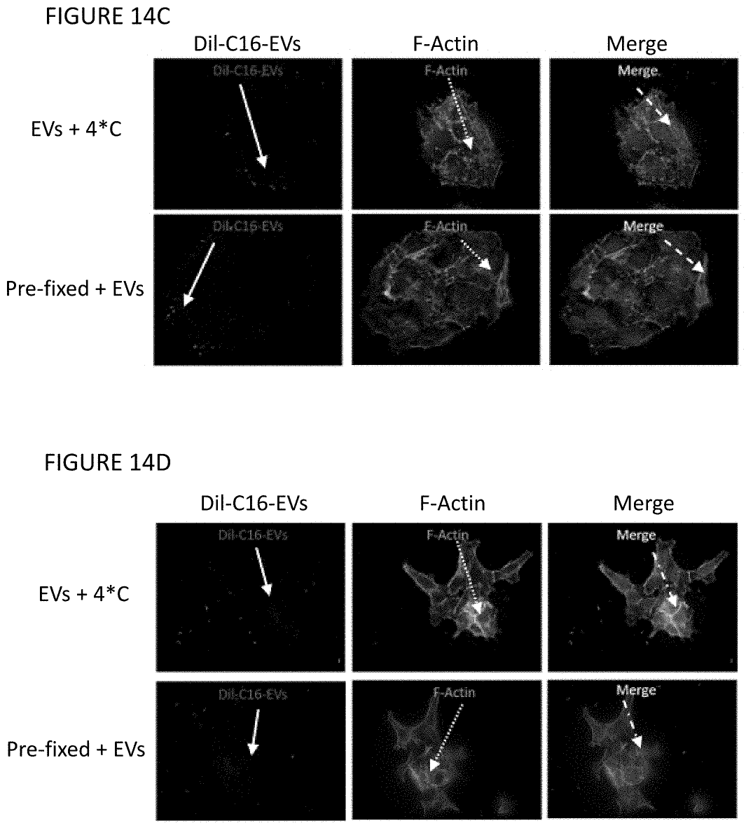

With respect to FIGS. 14A-14D, the bioreactor-derived EVs were labeled with Dil-C16 (lipophilic membrane stain) and incubated for 3 hours for cellular uptake. The red color indicates DiI-C16 labeled EVs, the green color indicates Phalloidin-Fluor 488 stained F-actin, and the blue color indicates DAPI stained nuclei. These images confirmed the efficient uptake of bioreactor derived EVs by HepG2 cells.

The bioreactor-derived EVs were labeled with Dil-C16 (lipophilic membrane stain) and incubated for 3 hours for cellular uptake. The red color indicates DiI-C16 labeled EVs, the green color indicates Phalloidin-Fluor 488 stained F-actin, and the blue color indicates DAPI stained nuclei. These images confirmed the efficient uptake of bioreactor-derived EVs by HCT116 cells.

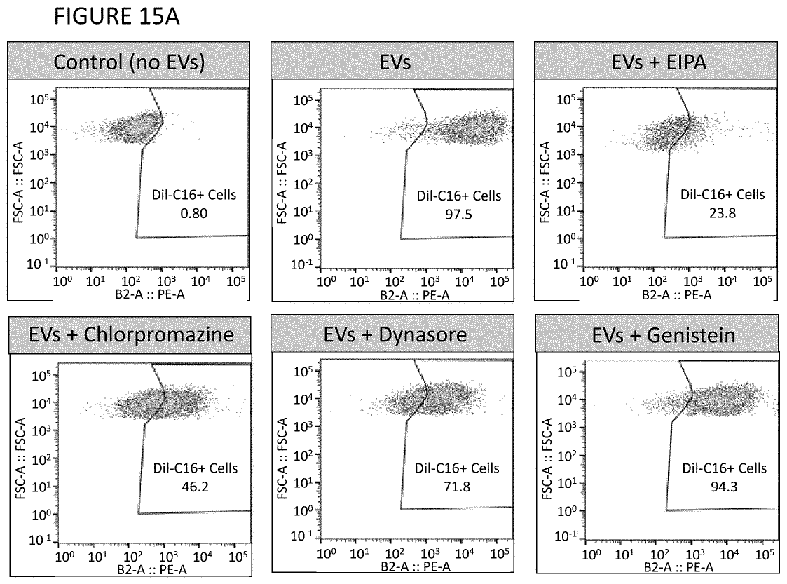

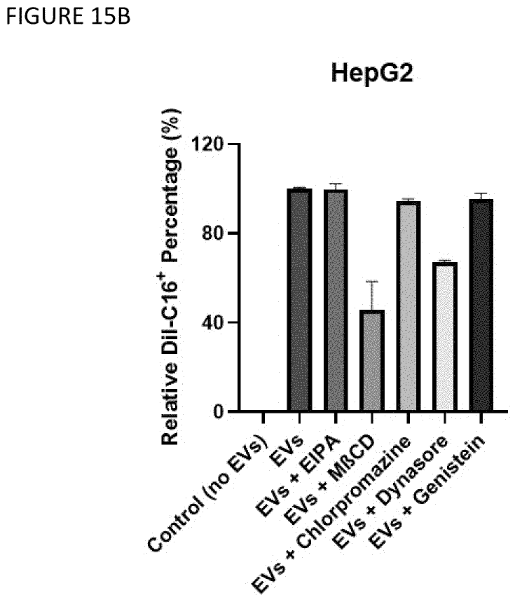

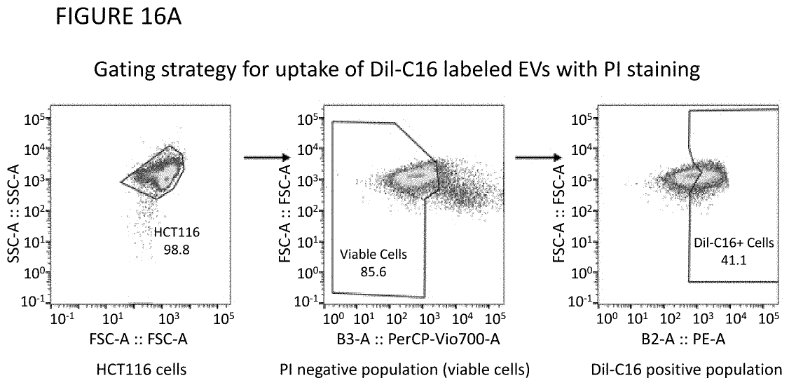

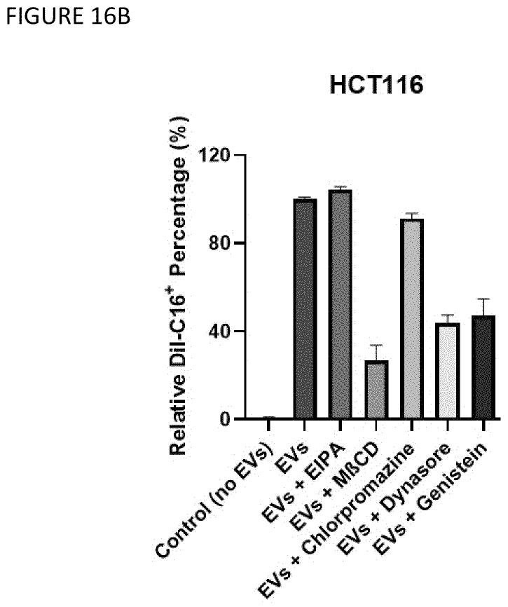

With respect to FIGS. 15A-15B and FIGS. 16A-16B, inhibitors of different internalization pathways were used to investigate the mechanism of bioreactor-derived EVs uptake by cancer cells. With respect to FIGS. 15A-15B, the HepG2 cells were pre-treated with different inhibitors for 1 hour, followed by co-incubating with DiI-C16 labeled EVs for 3 hours. The EV uptake was determined by the intensity of DiI-C16 using flow cytometry. The result suggested that HepG2 uptake bioreactor-derived EV through macropinocytosis, clathrin-dependent endocytosis, dynamin-dependent or independent endocytosis, but not through caveolae-dependent endocytosis.

In FIGS. 16A-16B, the HCT116 cells were pre-treated with different inhibitors for 1 hour, followed by co-incubating with DiI-C16 labeled EVs for 3 hours. The EV uptake was determined by the intensity of DiI-C16 using flow cytometry. To check the viability of HCT116 cells, the cells were stained by Propidium Iodide (PI) at the same time. FIG. 16A shows the gating of viable cells with PI staining (PI negative population). FIG. 16B shows the gating of DiI-C16 positive population. The result suggested that HCT116 uptake bioreactor-derived EV through lipid raft-mediated endocytosis, clathrin-dependent endocytosis, dynamin-dependent or independent endocytosis, and caveolae-dependent endocytosis.

FIG. 17A provides an example of a product that contains large numbers of microvesicles. Exosome activity is demonstrated by active uptake in vitro in HepD2 cells, which provides a potential for mixed engineered product that can provide 1) a delivery of a therapeutic protein secreted from PLCs and 2) an intracellular deliver of a siRNA as illustrated in FIG. 17B.

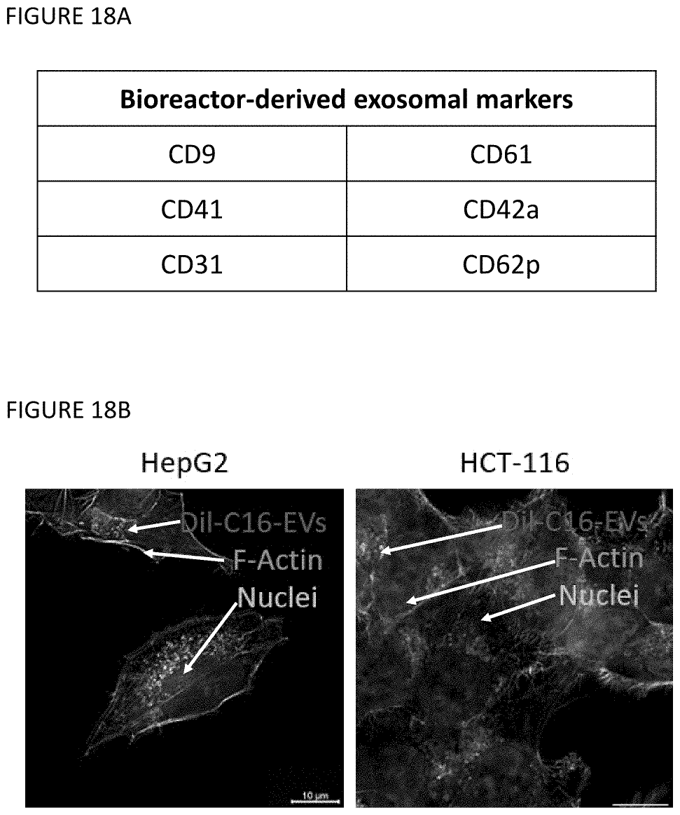

Results in FIGS. 18A and 18B demonstrate cancer cells take up the bioreactor derived exosomes. FIG. 18A illustrates some of the bioreactor derived exosome markers. Labelling and uptake of exosomes by cancer cells (HepG2 and HCT-116) is shown in FIG. 18B.

### Example 7:IL-12 Protein Expression is Upregulated in Engineered EVs Derived from PLC-Producing Progenitor Cells Exogenously Expressing IL-12

To determine whether protein of interest can be loaded into the PLC-EVs through the approach of molecular engineering, IL-12 is selected as a proof-of-concept protein. Engineered iPSCs (eiPSCs) expressing IL-12 were developed, followed by differentiation, and underwent Bioreactor run to generate IL-12 expressing engineered PLCs (IL-12 ePLCs). The EVs were isolated from the spent media during differentiation and the supernatant of the bioreactor run. Proteins were extracted from these EVs using RIPA (radioimmunoprecipitation assay buffer) buffer supplemented with protease inhibitor cocktails, then subjected to BCA assay for concentration determination. The protein amount was normalized to 20 μg for each sample, and the IL-12 protein concentration was measured using human IL-12 p70 ELISA kit (R&D Systems) as per the instructions.

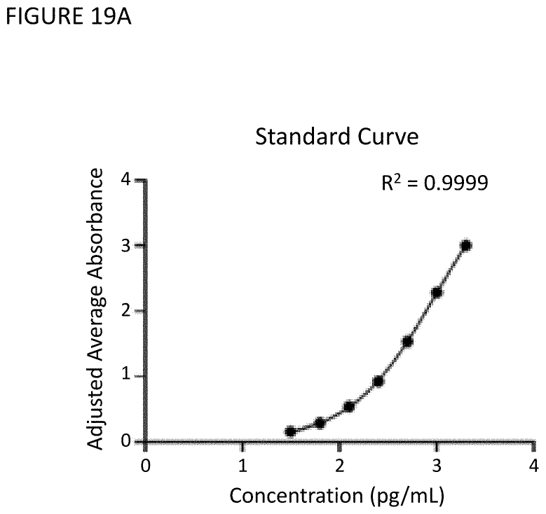

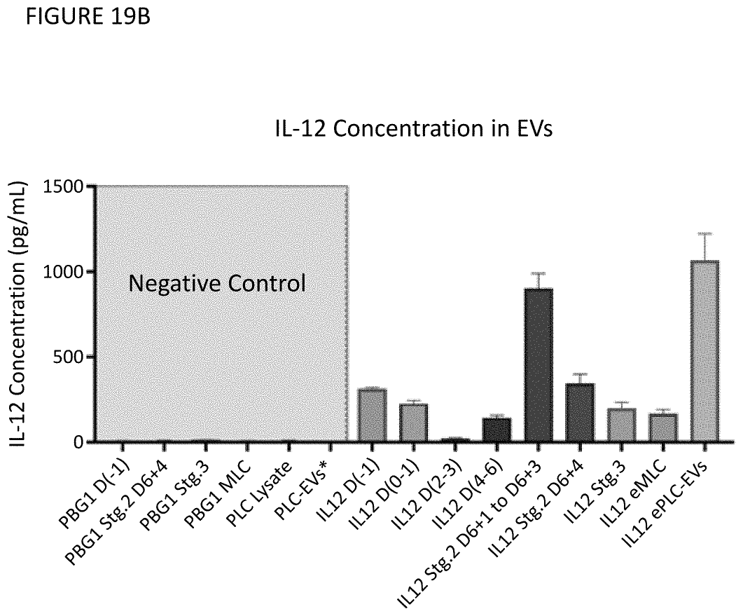

The results shown in FIGS. 19A and 19B indicate that the IL-12 protein expression was low or below detection limit in the EVs that were derived from PBG1 control cells (iPSCs, MLCs, and PLCs). On the other hand, the IL-12 expression was significantly elevated in the EVs (engineered EVs) that were isolated during the IL-12 eiPSC differentiation (FIG. 19B). Furthermore, the IL-12 expression in the IL-12 eMLC and IL-12 ePLC-EVs were also elevated, and the concentration was 169 and 1066 pg/mL, respectively (an example of measurement of IL-12 concentration is shown in FIG. 19A). This study demonstrated that by molecular engineering of iPSCs followed by differentiation and bioreactor run, the protein of interest (i.e., IL-12) can be efficiently loaded into the ePLC-EVs.

### Example 8: Exogenous Loading of siRNAs into PLC-EVs (EV-siRNA Uptake by HepG2 Cells)

To check whether siRNA can be encapsulated into PLC-EVs through exogenous loading approach, and whether PLC-EVs can be exploited as the delivery vehicle for siRNA to cancer cells, the siRNA conjugated with TX-Red dye was used for demonstration. 20 pmol Tx-red-siRNA was encapsulated into 50, 100, 200, and 300 μg PLC-EVs in the presence of Exo-Fect Exosome Transfection Reagent (System Biosciences, LLC) to determine the ratio of siRNA and PLC-EVs for optimized encapsulation efficiency. After loading of Tx-red-siRNA, the PLC-EVs were incubated with ExoQuick-TC reagent at 4° C. for 30 minutes, followed by spun down at top speed for 10 minutes, and resuspended in 200 μL PBS.

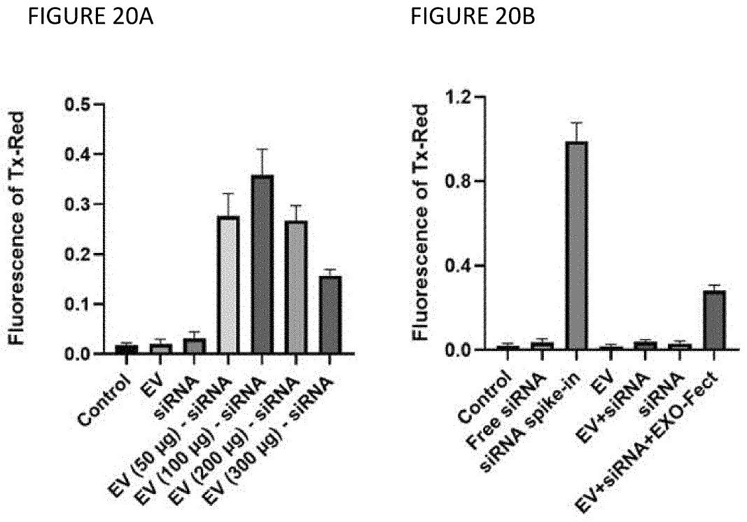

The PLC-EVs loaded with Tx-red-siRNA were then co-incubated with HepG2 cells for 3 hours. The amount of siRNA that was taken up by HepG2 cells was quantified by measuring the intracellular signal intensity of Tx-red. Briefly, HepG2 cells were seeded in 96-well plate, and 20 μL of the resuspension (equal to 2 pmol of siRNA) was added to each well. At the end of the study, the media was removed, the cells were washed twice with PBS, lysed by RIPA buffer at RT for 15 minutes, and the fluorescence intensity was measured by plate reader with the wavelength of 590/615 nm. The result showed that the ratio of siRNA/PLC-EVs at 1:5, which means 20 pmol of siRNA and 100 μg of PLC-EVs, rendered better intracellular delivery of siRNA (FIG. 20A).

To further check the delivery potential of PLC-EVs, a comparison between siRNA alone, EV+siRNA with or without the treatment of EXO-Fect transfect reagent was completed using the same approach. After 3 h incubation with HepG2 cells, the Tx-red intensity was measured by fluorescent plate reader. The result indicated that the intracellular Tx-red intensity was minimal for the groups incubated with siRNA alone or EV+siRNA without EXO-Fect transfection reagent, meanwhile, when the siRNA was loaded into PLC-EVs by EXO-Fect, the group receiving the PLC-EV loaded siRNA exhibited significantly increased intracellular Tx-red intensity (FIG. 20B). It demonstrated that free siRNA and siRNA without encapsulation into PLC-EVs cannot be internalized into HepG2 cells. Overall, we proved that siRNA can be exogenously loaded into PLC-EVs with high encapsulation efficiency and the PLC-EVs can be used as the delivery vehicle for siRNA to cancer cells.

### Example 9: EVs are Capable of Delivering Cargos to Target Cells: Imaging of Co-Localization of EVs and siRNAs in the HepG2 Cells

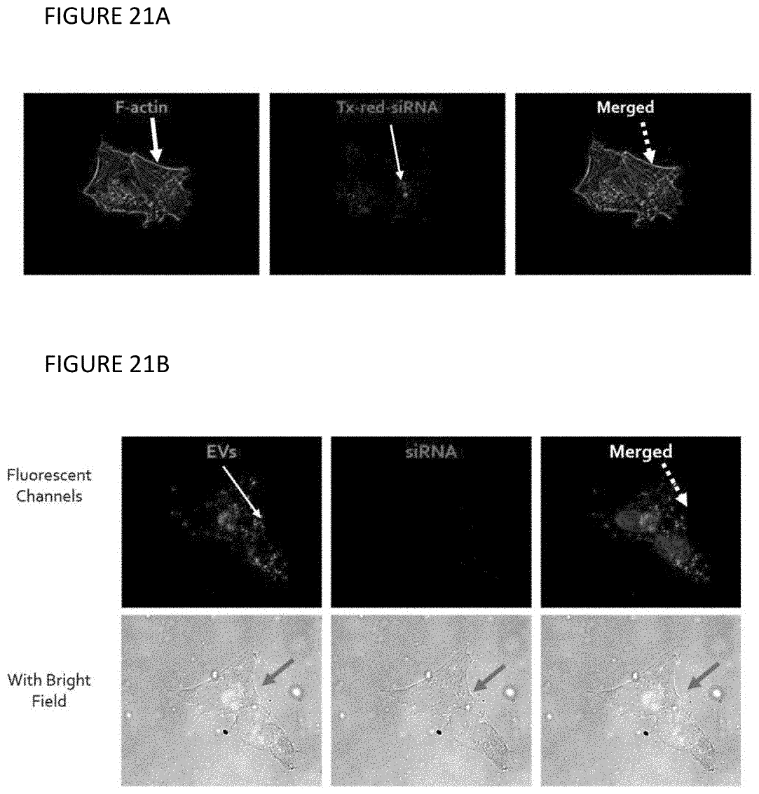

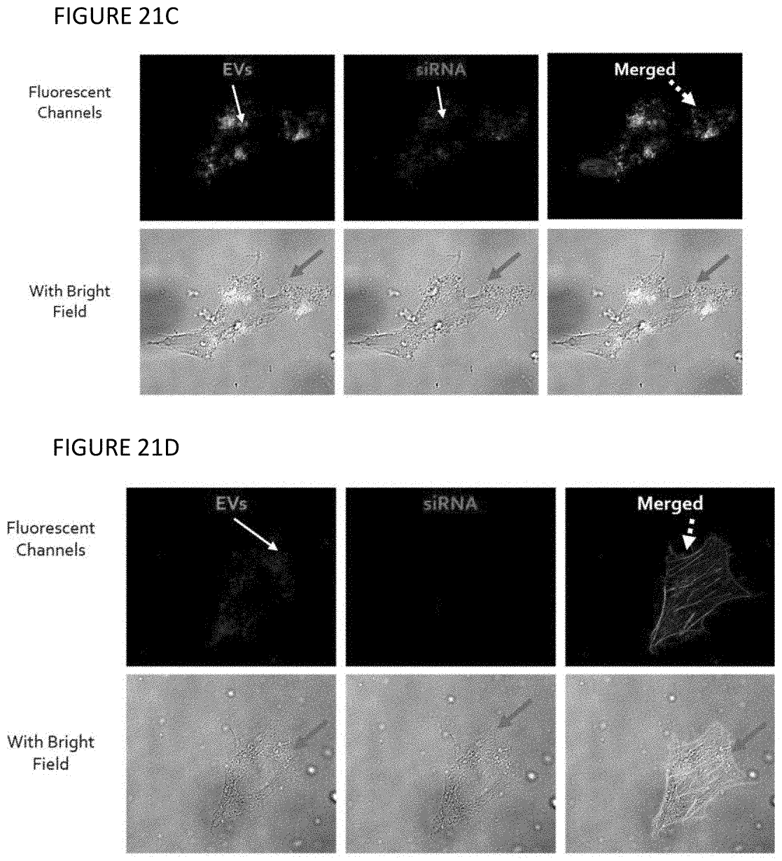

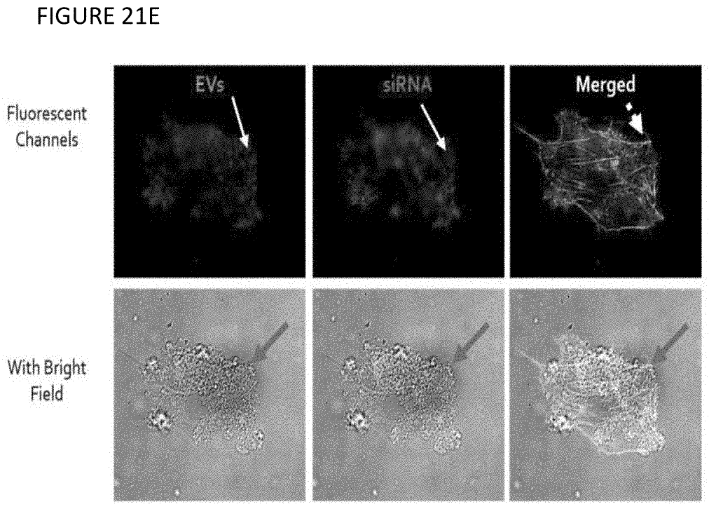

The internalization of siRNA into HepG2 cells was visualized by Thunder Microscope (Leica Microsystems) to further demonstrate the delivery capability of PLC-EVs. Using the above-mentioned encapsulation procedures to load Tx-red-siRNA into PLC-EVs, followed by co-incubation with HepG2 cells for 3 hours, the F-actin was further stained by Phalloidin-488 (green) to show the cell structures, and the cell nuclei were stained by DAPI (blue). The images showed the presence of a large amount of Tx-red-siRNA (red) in the cytoplasm of HepG2 cells (FIG. 21A).

To prove that PLC-EVs were able to load siRNA, and only siRNA that was encapsulated into PLC-EVs can be internalized into HepG2 cells, the intracellular co-localization of siRNA and PLC-EVs was also visualized. Two approaches were utilized: 1) green (PLC-EVs), red (siRNA), blue (nuclei); and 2) blue (PLC-EVs), red (siRNA), green (F-actin). The bright field was utilized to visualize whole cells for both approaches. The images showed that without EXO-Fect mediated encapsulation, there were only green dots (FIG. 21B) or blue dots (FIG. 21D) without red color indicating intracellular presence of PLC-EVs and absence of siRNA. On the other hand, after EXO-Fect mediated encapsulation, in FIG. 21C, both green and red dots presented in the cytoplasm of HepG2 cells indicating the internalization of PLC-EVs and siRNA, and the yellow color in the merged images indicated the co-localization of PLC-EVs and siRNA. Similarly, in FIG. 21E, the blue and red dots represented PLC-EVs and siRNA, and the purple color indicated the co-localization. These images proved that PLC-EV loaded siRNA can be efficiently uptake by HepG2 cells, and the siRNA that was not encapsulated cannot be internalized during the uptake of PLC-EVs.

### Example 10: siRNAs are Biologically Functional After Delivering to HepG2 Cells

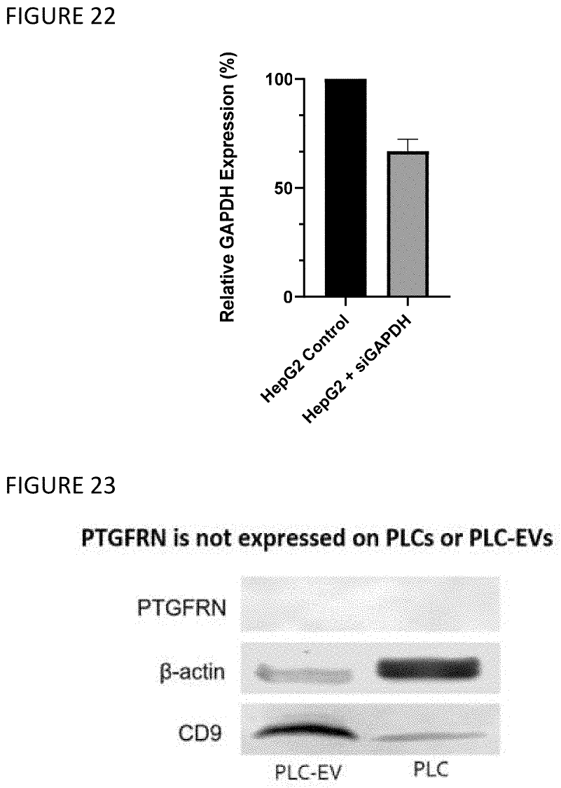

After demonstrating that siRNA can be encapsulated into PLC-EVs by exogenous loading approach, and PLC-EVs were able to deliver siRNA into cancer cells, whether the delivered siRNA was still biologically functional was checked using siRNA against GAPDH (siGAPDH). Briefly, siGAPDH was encapsulated into PLC-EVs which was mediated by EXO-Fect transfection reagent. 40 pmol of siRNA and 200 μg of PLC-EVs per well were added to HepG2 cells in 6-well plate for 24 h incubation time. At the end of the study, the cells were washed with PBS twice, followed by total RNA extraction. The total RNA concentration was measure by Nanodrop instrument to normalize the RNA amount per reaction to 100 ng. The expression of the target gene GAPDH and β-actin which was used as the housekeeping gene was determined by RT-qPCR. The results showed that the expression of GAPDH in HepG2 cells receiving 40 pmol siGAPDH after 24 hours was downregulated to around 70% compared to the control HepG2 cells (FIG. 22). It was therefore concluded that siRNA was still biologically functional after PLC-EV mediated delivery to cancer cells.

### Example 11: Assessment of Expression of PTGFRN Expression in PLCs and PLC Derived EVs

To check the expression of PTGFRN (prostaglandin F2 receptor inhibitor) protein on both PLCs and PLC derived EVs (PLC-EVs), Western Blot was performed before molecular engineering of iPSCs. PLCs and PLC-EVs were collected and lysed using RIPA buffer supplemented with protease inhibitor cocktails on ice for 30 minutes with occasional vortexing. The samples were centrifuged at the speed of 13,000 g for 20 min at 4° C. to collect the supernatant. The protein concentration was determined by bicinchoninic (BCA) assay and all the samples were normalized to same concentration. The PLC and PLC-EV lysate samples were reduced and denatured at 70° C. for 10 minutes with the addition of NuPAGE LDS Sample Buffer (ThermoFisher) (4×).

20 μg of total protein of each lysate sample was loaded and separated by SDS-PAGE (sodium dodecyl sulphate-polyacrylamide gel electrophoresis) gel, along with protein molecular weight ladder, for 70 minutes, followed by transferring the protein from the gel to a PVDF membrane and blocked with 5% milk in TBS buffer at 4° C. overnight. The primary antibodies including rabbit anti-PTGFRN (1:1000), rabbit anti-β-actin (1:1000), and mouse anti-CD9 (1:500) antibodies were incubated with the membrane at room temperature (RT) for 1 hour. After washing the membrane with TBST (tris buffered saline, 0.1% TWEEN 20) buffer for 4 times, the secondary antibodies against rabbit and mouse were then incubated at RT for another 1 hour, followed by washing with TBST buffer again. The membrane was scanned using Licor imaging system. The result indicated that the PTGFRN protein was not expressed on both PLCs and PLC-EVs, while the expression of β-actin control protein was higher on PLC cells than on PLC-EVs, and on the other hand, the expression of exosome marker CD9 was higher on PLC-EVs than on PLCs (FIG. 23). This result was also consistent with our RNA-Seq data regarding PTGFRN. Therefore, we concluded that our PLC-EVs were lack of PTGFRN expression.

### Example 12: Generating PLCs from Genetically Modified Premegakaryoctyes

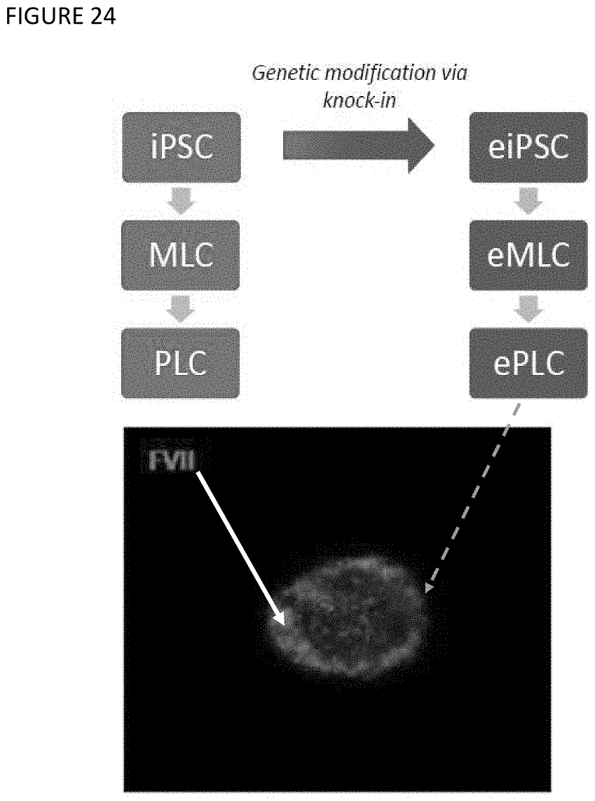

FIG. 24 shows PLC can be engineered (ePLC) to express specific antigens on its surface. The upper panel in FIG. 24 is a schematic representation of how ePLCs (hence EVs, as they are made as admixture with PLCs) are generated from iPSCs. The lower panel of FIG. 24 gives examples of such expression thereby proving the ability to express specific proteins, cytokines or monoclonal antibodies expressed alone or in combination from the same cell.

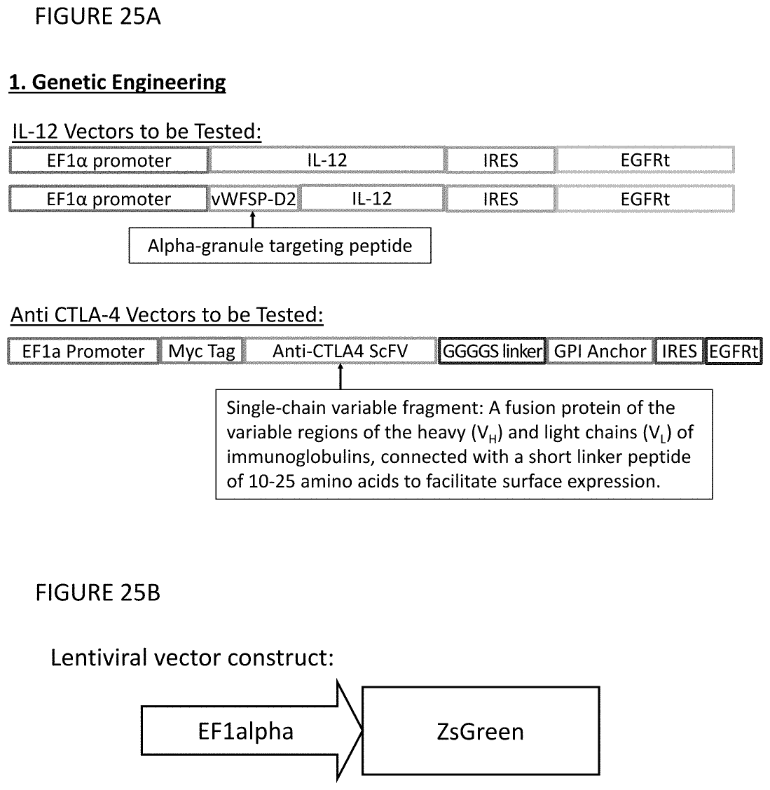

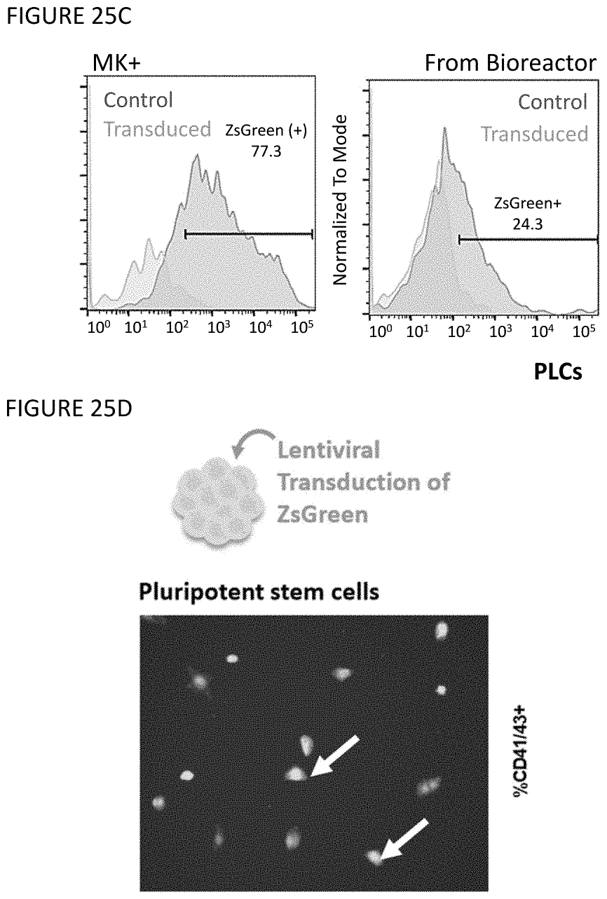

FIG. 25A shows DNA constructs designed to be incorporated into a vector that can be used for the generation of ePLCs to express IL-12 and an anti-CTLA4 ScFV. In some embodiments, PLCs expressing an exogenous DNA were generated from premegakaryocytes, which were transduced with a lentiviral vector comprising nucleic acid cassette encoding a reporter protein. Specifically, the cassette encoded an EF1alpha promoter and a ZsGreen fluorescent protein (FIG. 25B). 42 hours post infection with the lentiviral vector, fluorescence was detected in premegakaryocytes transduced but not in the untransduced indicating that the premegakaryocytes were successfully transduced (FIG. 25D). The premegakaryoctyes carrying the transgene were cultured according to the methods described herein to produce PLCs in a bioreactor. To validate that the fluorescent signal was produced by PLCs, the PLCs derived from the mock and the lentivirally transduced megakaryoctyes were sorted using a CD61 gating strategy. The fluorescent histogram shown in FIG. 25C demonstrates that the fluorescent signal was detected in CD61+ platelet like cells. In a manner discussed in this example, any transgene can be transduced into PLC producing progenitor cells, such as MKs, for the genes to be expressed in the PLCs. Two, non-limiting, examples of exogenous constructs are shown in FIG. 25A (genetically engineered IL-12 and genetically engineered anti-CTLA4ScFV). IL-12 or anti-CTLA4ScFV can be readily replaced with a gene of interest. Genetically engineered PLCs can be administered to a patient in an amount effective for the treatment of a disease or disorder that can be treated by a protein of interest produced by the transgenic PLCs.

### Example 13: ePLCs Expressing HGF: HGF Protein as Measured by ELISA is Increased in HGF

(A) Expressing Single Cell Clone G8 Generated from Transduced iPSC Populations.

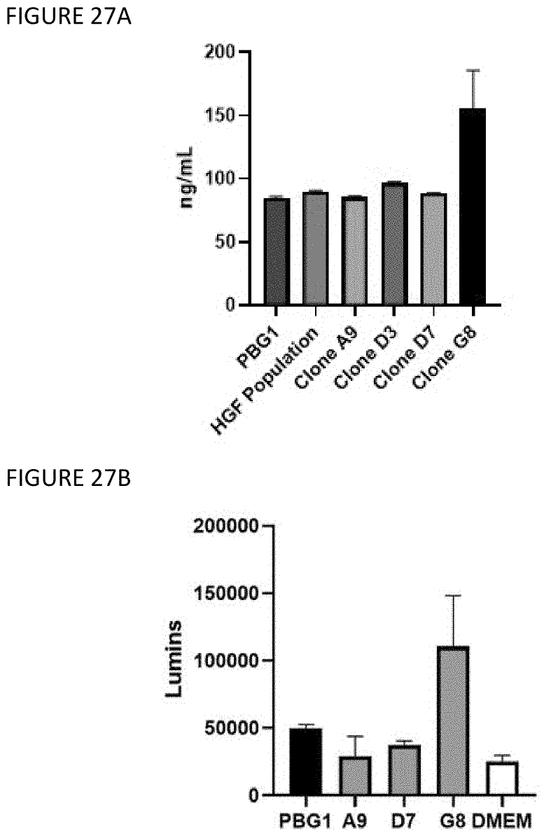

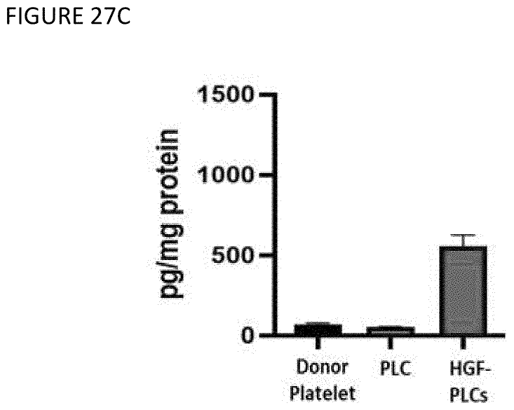

HGF protein quantitation as measured by ELISA (R&D Systems) was performed on cell culture supernatants derived from untransduced PBG1 cells, the antibiotic selected HGF expressing iPSC cell population and individual HGF expressing single cell iPSC clones (A9, D3, D7, G8). ePLC clone G8 showed the highest level of secreted HGF protein and was selected for further development (FIG. 27A).

(B) A Cellular Activity Assay Confirms Active HGF Protein Expressed from Clone G8.

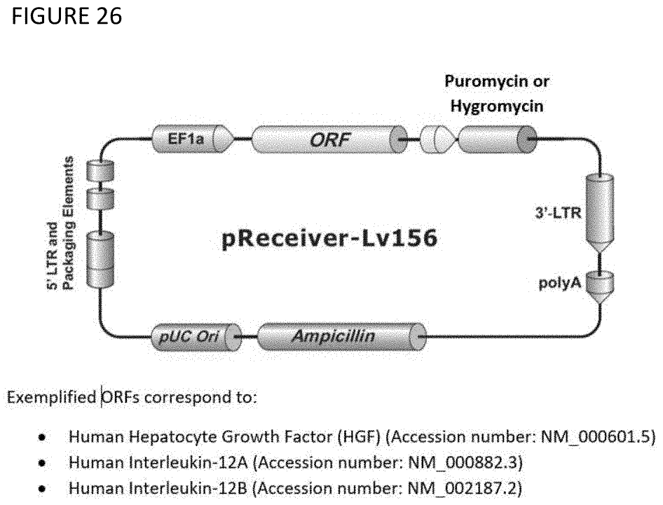

FIG. 26 is another illustration of a vector (pReceiver Lv156; Genecopoeia) that can be used to generate the ePLCs/eEVs. While the use of this vector is exemplified with exogenously expressing HGF, IL-12A and IL-12B, these genes can be readily replaced with other-genes of interest (for example a gene encoding a receptor, a ligand, a growth factor, an antibody or fragments thereof, a bacterial or viral protein, a biologically active toxin and any other biologically active protein or polypeptide). Quantification for the gene of interest can be performed as described here with the examples of HGF and IL-12. As an example, quantitation of HGF activity was performed using a cellular activity assay wherein STAT3 activation is monitored with luciferase. Media alone (DMEM) or cell culture supernatants from untransduced PBG1 iPSC or HGF transduced single cell iPSC clones (A9, D7 and G8) were analyzed in the activity assay (FIG. 27B). Clone G8 was observed to have significant activity as measured by luciferase signal compared to media alone or cell culture supernatants derived from PBG1 or clones A9 and D7 (FIG. 27B).

(C) HGF Protein is Increased in iPSC from Clone G8 and Throughout Differentiation.

PBG1 control (untransduced) and HGF expressing iPSC (Clone G8) were sampled throughout the differentiation process from iPSC through MLCs. Cell lysates in RIPA buffer were prepared from these cells and subjected to ELISA-based HGF protein quantitation. Results show elevated HGF protein levels in the transduced cells compared to PBG1 control at the iPSC stage, Stage 2 and 3 of differentiation and finally at the MLC stage. An approximately 5-fold increase in HGF protein is observed in MLCs compared to the control PBG1 cells (FIG. 27C).

(D) Expression of HGF in ePLC (HGF-PLCs) in Comparison to Donor Platelets and Untransduced PLC.

HGF protein was quantified by ELISA in cell lysates prepared in RIPA buffer from donor platelets, PLCs (untransduced) and HGF expressing PLCs. HGF protein amounts in the HGF-PLC lysate were significantly elevated compared to both donor platelet and PLC samples (FIG. 27C).

### Example 14: ePLC Expressing IL-12

(A) IL-12 Protein is Elevated in the IL-12 Transduced Cell Population Compared to PBG1 Control (Untransduced).

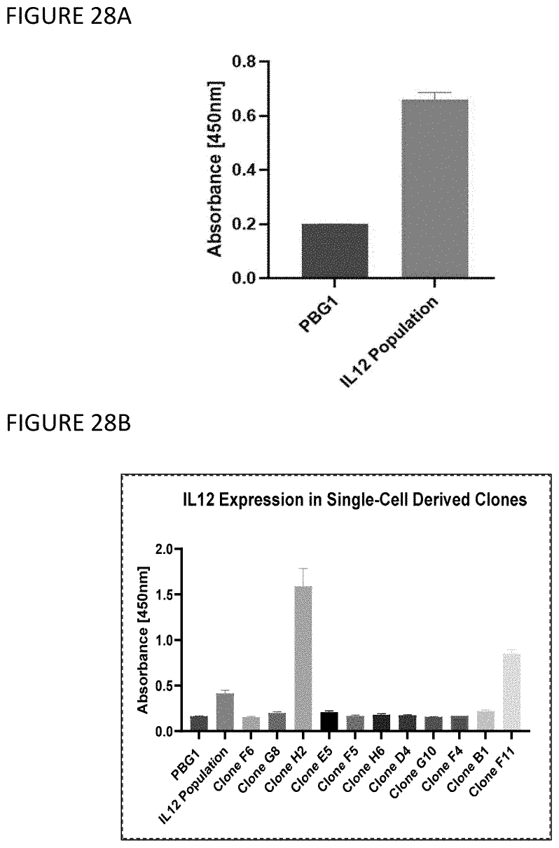

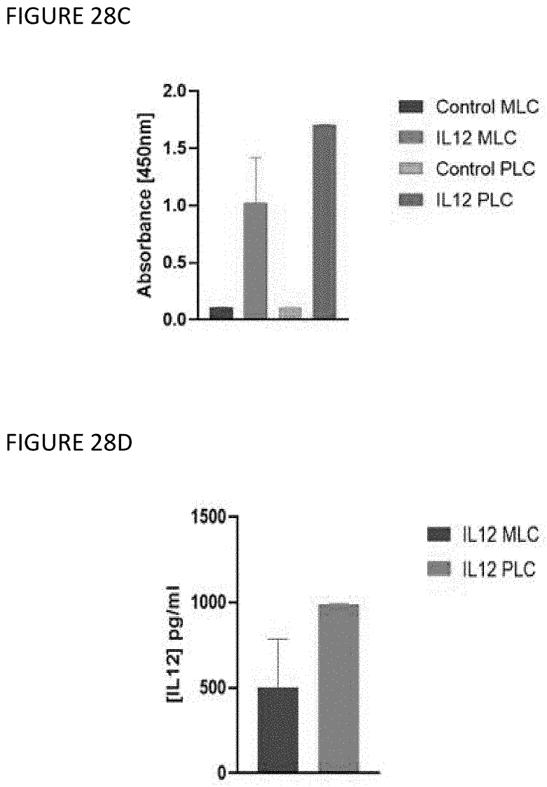

IL-12 heterodimer (p70) was quantified by ELISA from cell lysates prepared from PBG1 untransduced control cells or the antibiotic-selected cell population transduced with IL-12A and IL-12B lentivirus. A significant increase in IL-12 protein (p70) is observed in the transduced cell population compared to the PBG1 control (FIG. 28A).

(B) IL-12 Protein Levels in Single Cell Derived Clones Shows High IL-12 Expression from Clone H2.

IL-12 protein levels were quantified by ELISA in PBG1 untransduced control cells, the antibiotic-selected IL-12 population cells and individual single cell iPSC clones grown from the IL-12 transduced population. Increased IL-12 protein was observed in 2 clones, H2 and F11 (FIG. 28B). Clone H2 was selected for further development.

(C) IL-12 Protein Levels in the H2 Clone Differentiated to MLC and PLC.

Quantitation of IL-12 protein in control (untransduced) PBG1 differentiated cells and cells differentiated from the IL-12 expressing line (H2) was performed by ELISA on cell lysates. Increased IL-12 protein in the H2 line is observed in both MLC and PLC compared to control cells (FIG. 28C). Quantitation of the H2 protein based on the assay standard curve indicates protein quantities in the picogram range for MLC and near nanogram range for PLC (FIG. 28D).

### Example 15: PLCs can be Genetically Engineered to Express Multiple Proteins

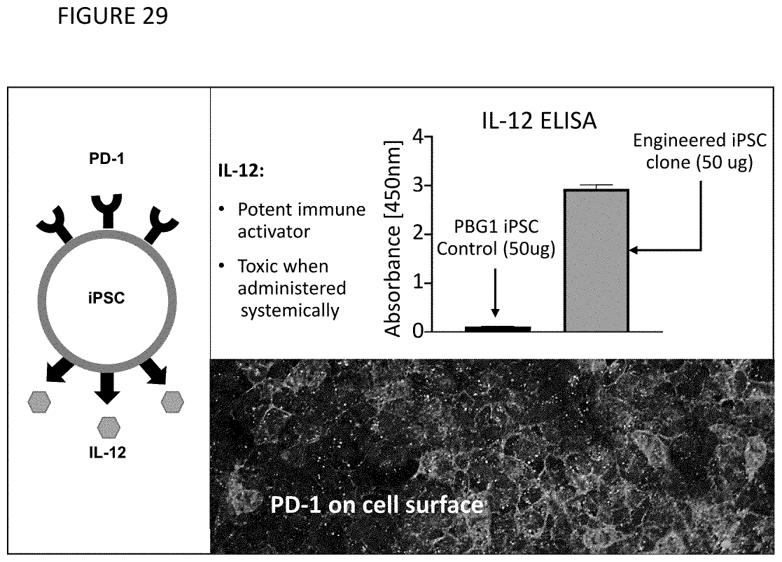

The left panel of FIG. 29 depicts a diagram of the genetically engineered iPSC clonal cell line that expresses two proteins of interest, PD-1 and IL-12. For PD-1 expression, PD-1 (accession number NM 005018.2) was expressed in LPP-B0169-Lv156 vector (Genecopoeia).

Right Top of FIG. 29: In order to quantitate IL-12 protein levels in the PD-1/IL-12 iPSC clonal cell line, lysates of the engineered cells were subjected to an IL-12 ELISA assay recognizing the functional p70 IL-12 heterodimer. The iPSC line expressing PD-1/IL-12 (right column) shows a high level of IL-12 protein compared to the non-transduced PBG1 control iPSC cell line (left column).

In order to confirm the surface expression of PD-1 protein on the genetically engineered iPSC clonal cell line, immunofluorescence analysis was performed using an anti-PD-1 antibody (green) with DAPI staining (blue) to confirm nuclear staining. A strong PD-1 signal is confirmed in the cell line that appears to be distinct from the nucleus (Right Bottom of FIG. 29).

### Example 16: PLCs Reduce Liver Fibrosis in a Mouse Disease Model

In order to determine if PLCs have a beneficial effect in treating liver fibrosis, fibrosis was initiated with 3× weekly application of 0.1 mL/kg injection of carbon tetrachloride diluted in corn oil for 2 weeks; control mice received corn oil itself for those 2 weeks. Carbon tetrachloride applications continued throughout the next step. Mice that received carbon tetrachloride were separated into 3 cohorts at the two-week mark:
1. Plasmalyte application on D15, 22, 29 (Carbon Tet mice)
2. rHGF diluted to 30 ug/mL in plasmalyte and dosed daily at 0.3 mg/kg (Peprotech HGF 100-39H-1 mg)
3. PLC applications of ˜3e10 Platelet Equivalency Units (PEUs)/kg per mouse on D15, 22, 29. D15 was 5e10, D22 was 3e10, D29 was 5e10.

All mice were sacrificed on D30, livers were excised, and fresh frozen for ˜2-3 hours. Hydroxyproline analysis was done using a hydroxyproline kit (Abcam #ab222941).

Briefly, livers were trimmed, weighed, and the homogenized in DI water at ˜10 uL water/mg of liver with a Dounce homogenizer. The equivalent of 10 mg (˜100 uL) were removed and digested in equal volume 10N NaOH for 2 hours at 120° C. Samples were cooled and quenched with 100 uL 10N HCl, vortexed, and then spun in a centrifuge for 5 mins at 10,000×g. 10 uL and 100 uL samples were transferred to a 96-well plate and evaporated. Samples were then processed as the manufacturer directed and analyzed with a plate reader. Concentrations were based off of standards provided in the kit.

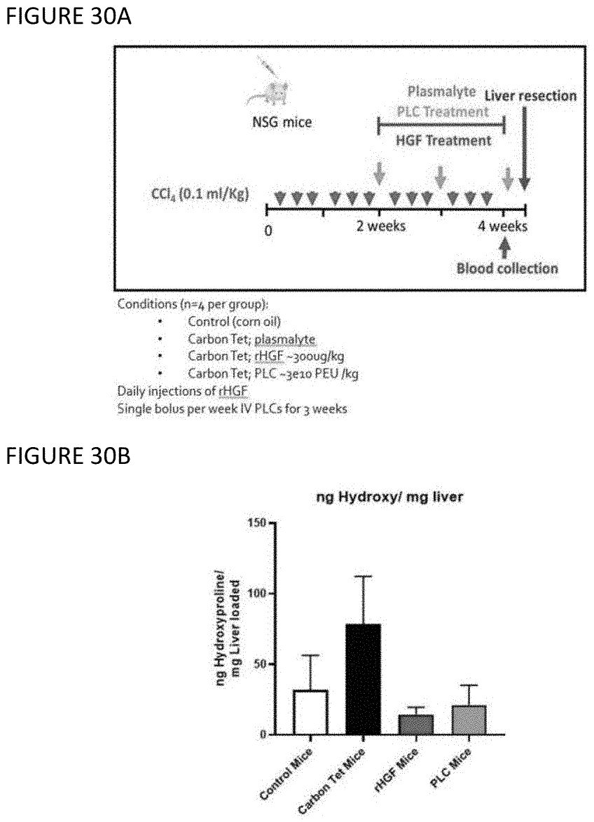

As shown in FIGS. 30A and 30B, there is a carbon tetrachloride-dependent increase of collagen in livers of these mice and an apparent reduction of collagen per weight of liver of those mice treated with either HGF or PLCs as compared to those that received no intervention (Carbon tet mice). Therefore, PLCs can effectively reduce liver fibrosis or may impact other diseases where HGF has a role to play.

### Example 17: High Levels of HGF are Expressed in ePLC

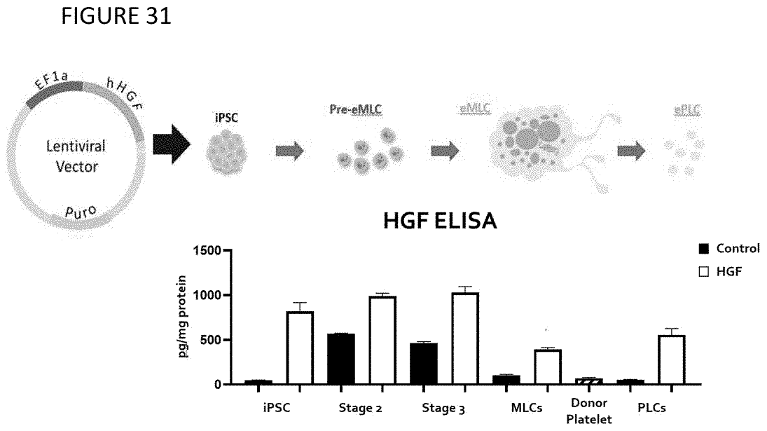

PB101 iPSC were transduced with an EF1α-promoter-driven HGF-expressing plasmid shown in FIG. 31, selected with antibiotics, single-clone isolated, and expanded. PB101 iPSC and HGF-expressing clones (HGF; also referred to as engineered, or eCells) were differentiated and isolated through the previously described stages: iPSC, Stage 2, Stage 3 (pre-freeze), MLC (post-thaw), and PLC production. Cells were lysed in 1× RIPA buffer (EMD Millipore Cat #20-188), protein content was quantified with Pierce 660 assay (Thermo Scientific Cat #22660), loaded into an ELISA assay (Both R&D Cat #DHGOOB and Abcam Cat #ab100534 were used), and data was analyzed and standardized to protein input. HGF-expressing clones consistently were found with an average of −5-fold increases in HGF when compared to PB101 cells (FIG. 31).

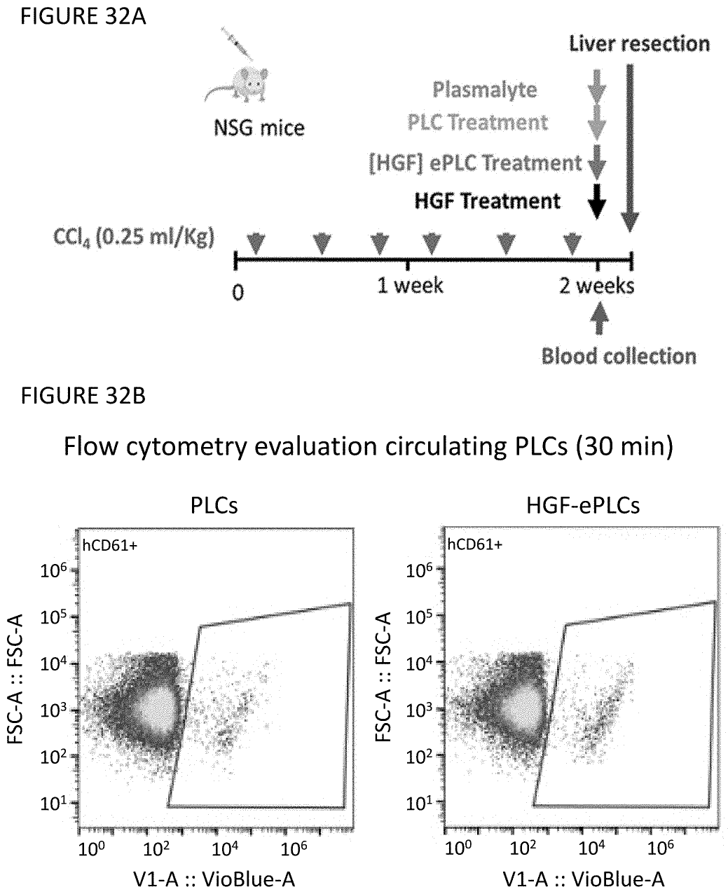

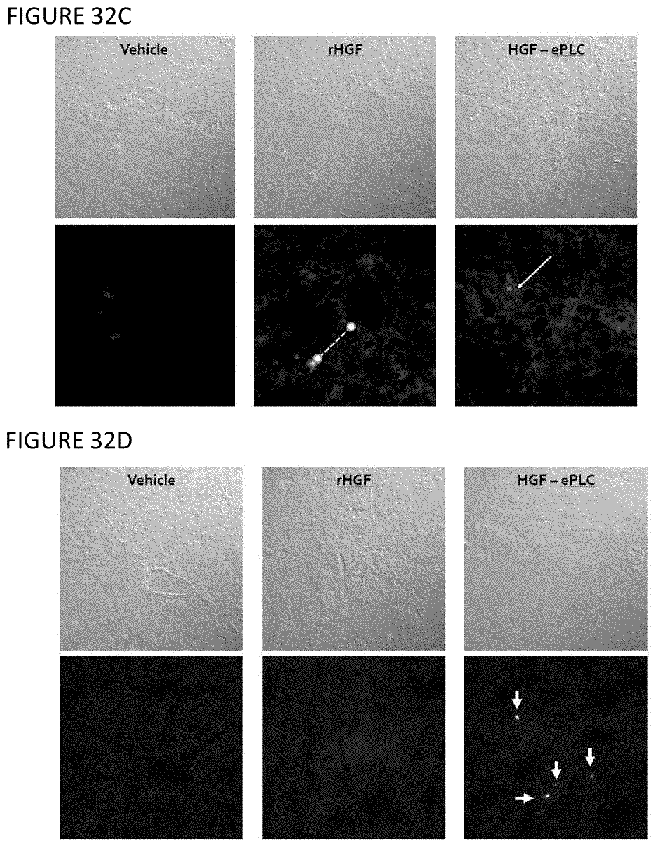

FIG. 32A shows experimental plan for in vivo localization. Immune-deficient mice (NSG) were dosed with 0.25 mL/kg of carbon tetrachloride 3 times per week for 2 weeks to initiate liver fibrosis. Control mice were treated with similar volumes of corn oil. On D15, fibrotic mice were treated with the following: Plasmalyte, recombinant HGF (Peprotech Cat #100-39H-1 mg), PLCs that had been generated the previous day and diluted in plasmalyte, or HGF-ePLCs that had been generated the previous day and diluted in plasmalyte. Treatments were allowed to circulate for 30 mins and then mice were sacrificed, exsanguinated, and livers were prepared and frozen for analysis.

FIG. 32B examines circulating PLCs in blood. Blood from PLC or ePLC treatment mice was analyzed via flow cytometry with a human-specific CD61 antibody (Miltenyi Cat #130-110-754) as an indicator of circulating PLCs/ePLCs. Approximately equal numbers of circulating PLCs and ePLCs were observed in the blood of mice. In FIG. 32C, fluorescence staining of livers removed from treated fibrotic mice for HGF were performed. Livers were embedded in OCT, sectioned, and mounted on slides. Sections were then fixed in ice-cold Acetone for 10 mins and dried for an additional 20 mins. Sections were blocked in 10% goat serum (Life Technologies Cat #50062Z), incubated with primary antibody directed towards human HGF (R&D Cat #MAB294), washed with PBS 3 times, and then incubated with an anti-mouse antibody conjugated to alexafluor-647 (Invitrogen Cat #A-21235). Images were obtained using a Leica THUNDER imaging system. Both recombinant HGF and ePLC treatments appear to increase HGF signal in the livers of mice. The results shown in FIG. 32D show fluorescence staining of livers removed from treated fibrotic mice for CD61. Slides from (FIG. 32C) were treated in the same way up to blocking step. Slides were incubated with an anti-CD61 antibody (BioLegend Cat #336402), washed 3 times with PBS, and then incubated with an anti-mouse antibody conjugated with alexafluor-488 (Invitrogen Cat #A32723). Images were obtained using a Leica THUNDER imaging system. Specific human CD61 puncta can be observed in the ePLC treated livers, indicating at least a subset of ePLCs localized to the liver.

### Example 18: For Generation and Characterization of Engineered Platelet-Like Cells (ePLC) Expressing FVII

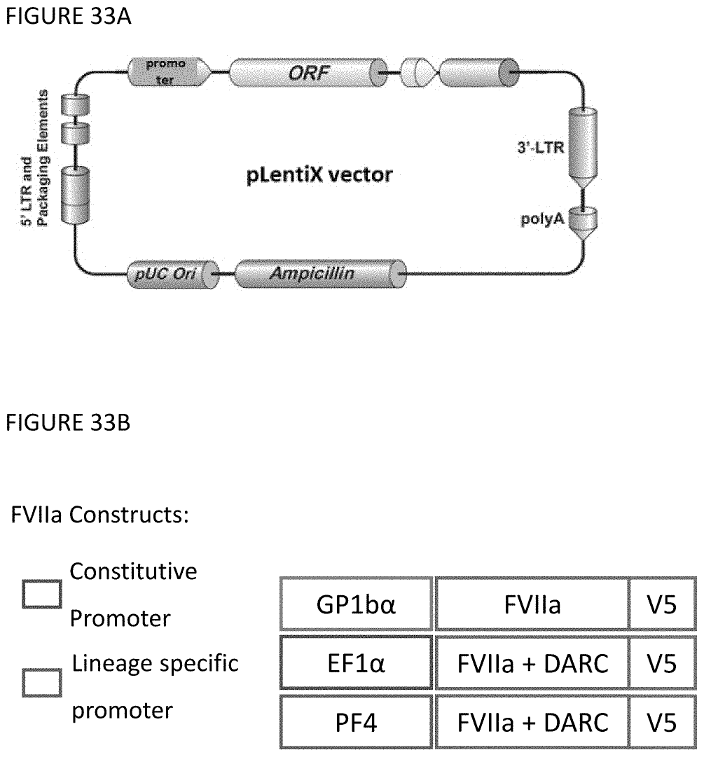

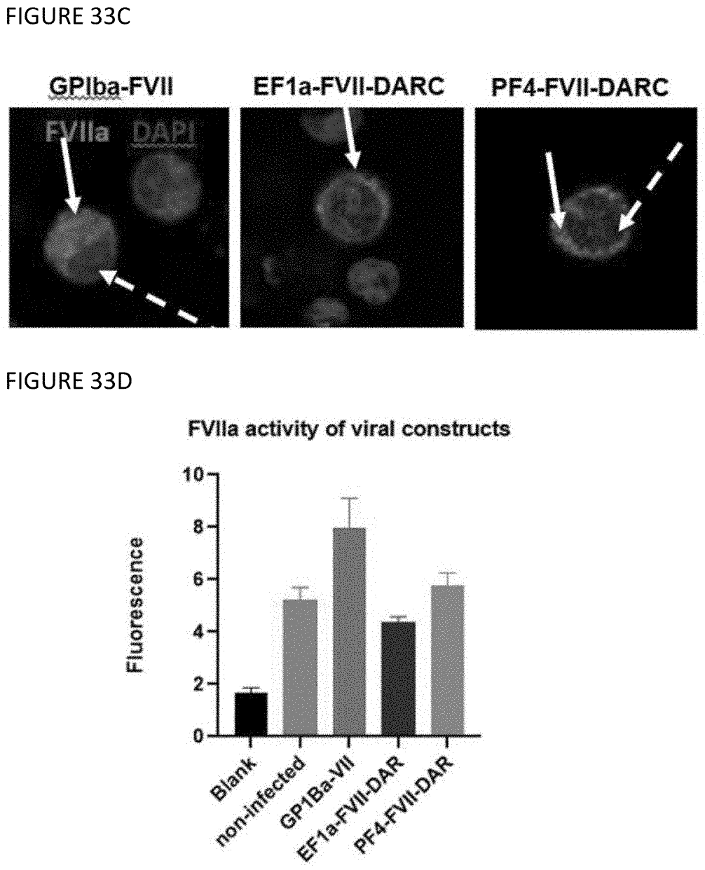

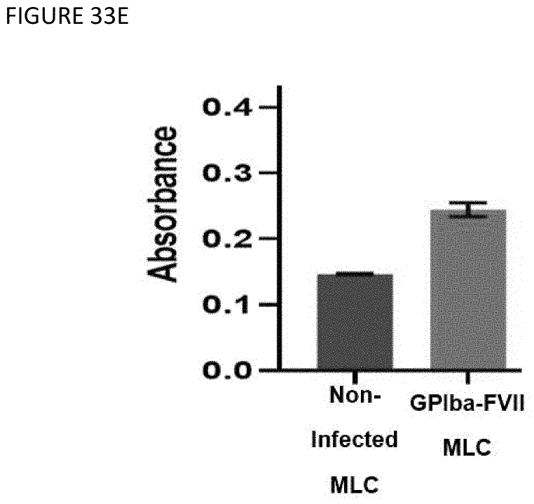

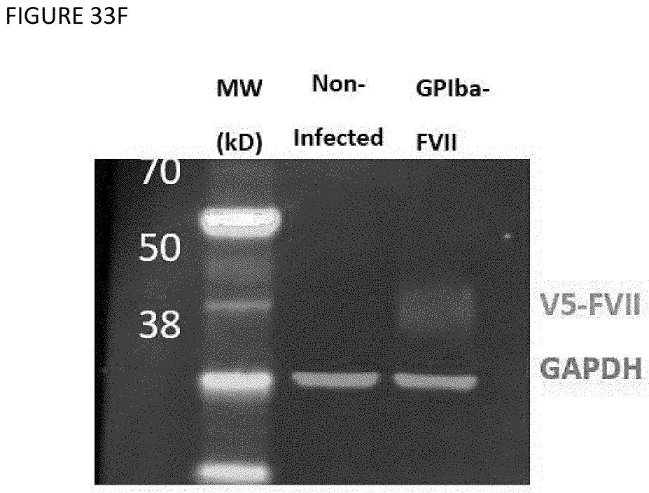

In order to generate and characterize ePLC expressing FVII, lentiviral particle supernatants were generated from packaging of lentiviral vectors (Thermo Scientific) that contain the open reading frame (ORF) of the FVII gene (Accession number: NM_019616). The constructs contain the FVII sequence engineered to contain a furin cleavage site (2RKR) into the factor X activation-cleavage site to allow for intracellular processing that results in activation of the FVII enzymatic activity. FVII was also engineered to express a V5 epitope on the C-terminus. Certain constructs are also designed to express FVII with the Duffy Antigen Receptor for Chemokines (DARC), a transmembrane protein to allow for membrane localization of FVIIa. Lentiviral vectors were further designed to have various promoters including EF1a, GP1bα, and PF4 drive expression of FVIIa (FIGS. 33A and 33B).

Lentiviral Transduction of preMLC to Achieve FVIIa Expressing Engineered MLC (eMLC) and Engineered PLC (ePLC)

Pre-megakaryocyte-like cells (pre-MLC) were thawed at 37° C. and gently resuspended in media. Cells were centrifuged at 300×g for 5 minutes, resuspended in media and plated at a concentration of 2×10⁶ cells per milliliter in a gas permeable rapid expansion (G-Rex) device. Cells were allowed to recover for 1-2 hours in a 37° C., 5% CO₂ incubator. For lentiviral infection, the cells were collected and lentiviral supernatant added to achieve the desired multiplicity of infection (MOI) based on the viral titer. MOIs tested ranged from 5-200. Cells were centrifuged at 300×g for 3 hours, then resuspended in the same media containing viral particles. Cells were plated in a G-Rex with stage appropriate media and incubated for 3 days with media replenishment as needed. Following 3 days of incubation, the majority of media was removed from the G-Rex and cells were collected and counted. eMLC were centrifuged at 120×g for 5 minutes prior to resuspension in appropriate media or buffer for further experimentation.

For in vivo eMLC infusion, 1×107 live cells were resuspended in 200 uL of plasmalyte or appropriate buffer for intravenous infusion into a single mouse.

For production of ePLC, 4×107 total eMLCs per layer were seeded into a bioreactor and subjected to shear flow to generate ePLC according to the standard bioreactor protocol.

Protein Analyses: Image Analysis of FVIIa Expression:

Cellular imaging was used to assess FVIIa expression in transduced MLCs. Fixed and permeabilized transduced MLCs were labeled with an anti-V5 antibody conjugated to a red fluorescent fluorophore was used to visualize expressed FVIIa (solid arrow) while DAPI was used to identify nuclear staining (broken arrow). FVIIa expression was visualized in MLCs from all three transductions attempted (FIG. 33C).

FVIIa Activity Assay

A quantitative assay to measure the activity of FVIIa in transduced MLC was developed based on cleavage of a FVIIa-specific fluorogenic substrate (SN17C, Haematologic Technologies). Activity of MLCs was compared to a standard curve based on concentrations of purified FVIIa (Haematologic Technologies). Transduced MLCs were resuspended in buffer and 100 uL of cell suspension was added to duplicate wells of a 96 well microwell plate. SN17C fluorogenic FVIIa substrate was added to each well to achieve a final concentration of 100 uM and incubated in the dark for 30 minutes. Wells of the microplate were measured in a fluorescence microplate reader at a excitation wavelength of 352 nm and an emission wavelength of 470 nm. MLC transduced with FVII lentivirses and non-infected control MLCs were analyzed in the assay. Increased FVIIa activity was observed for MLC transduced with the GPIba-driven FVIIa construct (Column 3 from left; FIG. 33D) as compared to non-infected MLC (Column 2 from left; FIG. 33D). FVIIa constructs fused to DARC (Columns 4 and 5 from left, respectively; FIG. 33D) did not exhibit increased FVIIa activity over the non-infected control.

FVII ELISA: To quantify FVIIa protein levels in transduced MLC, cell lysates were subjected to enzyme-linked immunosorbent assay (ELISA) using a commercially available FVIIa ELISA assay (Abcam). Briefly, MLC transduced with the GPIba-FVIIa construct of non-infected MLCs (termed MLCs) were lysed in radioimmunoassay precipitation (RIPA) buffer. Cell lysates were added to wells of the microplate assay in duplicate and assayed according to the manufacturer's directions. Following analysis, an increase in FVIIa protein level as reflected by increased absorbance in the assay is observed for the GPIba-FVII transduced MLC (right; FIG. 33E) in comparison to non-infected MLC (left; FIG. 33E).

Western blot analysis: Western blot analysis was performed to determine protein expression of FVII in transduced MLC in comparison to non-infected MLC. Briefly, MLCs transduced with the GPIba-FVII lentiviral supernatant or non-infected MLCs were lysed in SDS sample buffer and subjected to SDS-PAGE and Western blot analysis. An antibody to the V5 epitope tag was used to identify FVIIa protein and an antibody to GAPDH was used as an internal loading control (thin arrow; FIG. 33F). A protein band is observed in the GPIba-FVII MLCs while no protein band is observed in the non-infected cells, as indicated by a thick arrow in FIG. 33F, confirming successful lentiviral transduction in the transduced cells.

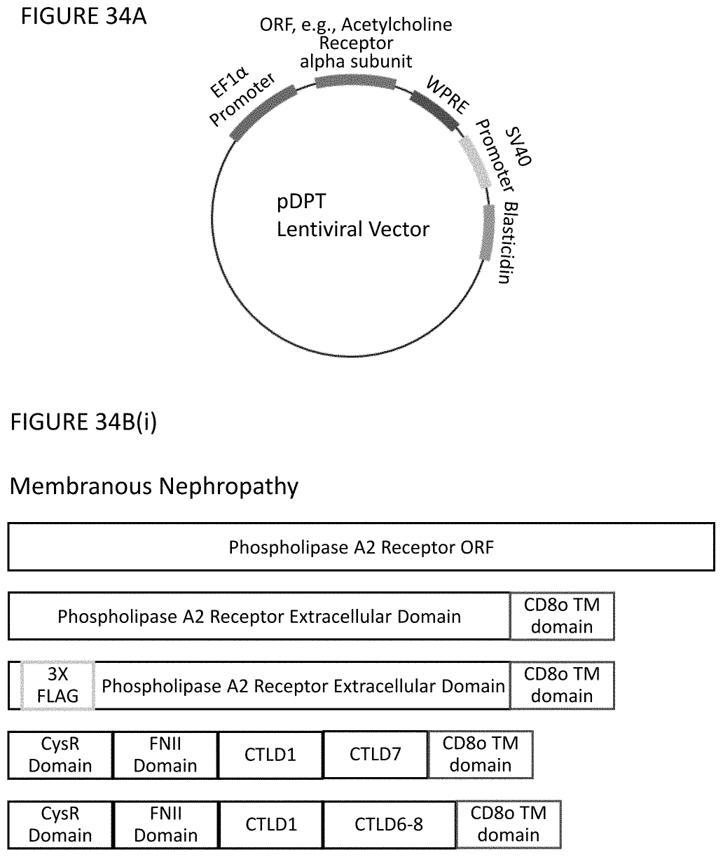

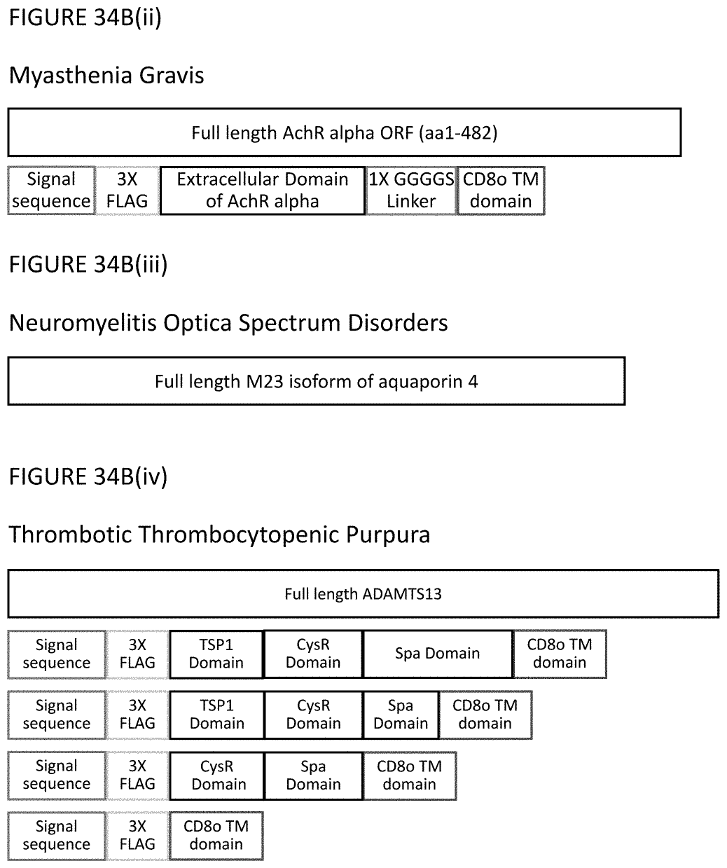

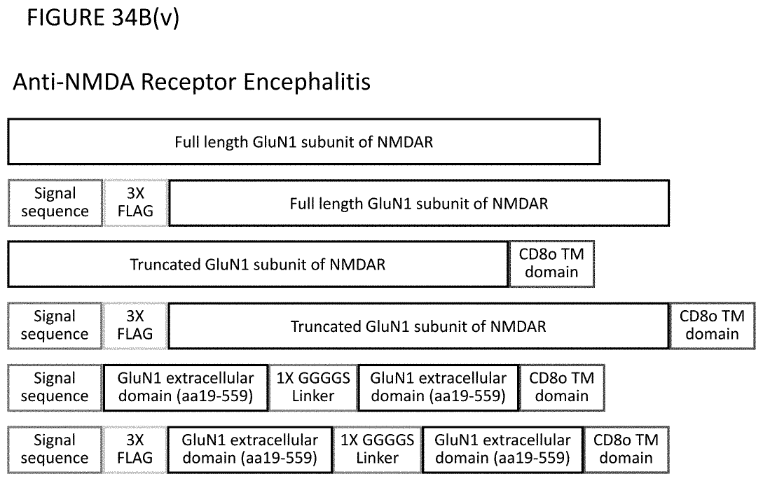

FIGS. 34A-34B(i-v) show examples of some of the genes that can be genetically engineered into the PLCs, the expressions of which can be characterized in the same manner as described in FIGS. 33A through 33F.

### Materials and Methods

Microfluidic Preparation and Adhesion Studies:

Equine Type I collagen was patterned into an Ibidi Slide VI 0.1 chip at 100 ug/mL one day prior to the assay. The excess collagen was rinsed, and the surface was blocked with 2% bovine serum albumin in phosphate buffer saline for 1 hour prior to the assay and the chip connected to a syringe pump with a Hamilton Gastight syringe (500 μL)

Blood Preparation:

Whole blood was collection via venipuncture into vacutainers containing sodium citrate. The vacutainers were centrifuged at 150 G for 17 minutes to separate the red blood cells (RBC) from the platelet rich plasma (PRP). PRP was collected, and the buffy coat was discarded. One milliliter of the PRP was set aside, and the remaining fraction was centrifuged at 2200 G for 20 minutes to pellet the platelets and the platelet poor plasma (PPP) fraction was collected. The RBC fraction was centrifuged at 1000 G for 5 minutes to pack the RBC and the top layer of plasma was discarded. Platelet counts were performed on the RBC, PRP and PPP fractions with a flow cytometer. The blood was then reconstituted to 100 aliquots of the desired conditions by mixing the appropriate amounts of the fractions (e.g., 26 μL RBC, 5 PRP, 69 μL PPP for a thrombocytopenic condition) Samples were then stained with Cell Tracker Deep Red (1 μM) for 20 minutes at 37 C immediately prior to perfusion. Results from this study is shown in FIGS. 3A-C.

For exosome-related study: Total Exosome Isolation Reagent (from cell culture media) (4478359), Exosome-Human CD9 Isolation Reagent (from cell culture) (10614D), DiR (D12731), and DiI-C16 (D384) were purchased from Invitrogen;

MACSPlex Exosome Kit, human (#130-108-813) was purchased from Miltenyi Biotec;

EIPA (A3085-25MG), MβCD (C4555-1G), Chlorpromazine (C8138-5G), Dynasore (D7693-5MG), and Genistein (G6649-5MG) were purchased from Sigma-Aldrich, Inc.;

Wherever applicable, HepG2 cells (HB-8065) were purchased from ATCC; HCT116-Fluc-Puro and HCT116-Egfp-Puro were purchased from Imanis Life Sciences.

EPLCs

Lentiviral Transduction of PBG1 and Antibiotic-Based Selection:

The PBG-1 iPSC cell line was grown to allow for a single cell suspension of 1×10⁶ cells/mL. Cells were transduced with lentivirus in the presence of 5 ng/mL polybrene at a multiplicity of infection of 10. To generate IL-12 expressing cells, a co-transduction was performed with addition of both IL-12A and IL12-B lentiviral supernatants to allow expression of both IL-12 subunits, necessary for proper protein function. Following addition of virus to cell suspensions, cells were incubated at room temperature for 15 minutes, plated and grown in normoxic conditions at 37° C. for 24 hours. Cells were then washed to remove virus and plated in fresh media. Antibiotic selection was used to select for transduced cells with integrated constructs. Cells were grown in selection media containing puromycin (1 μg/mL) for the HGF transduction or puromycin (1 μg/mL) and hygromycin B (500 μg/mL) for the IL-12 co-transduction for several days. The resulting iPSC population was evaluated for protein expression of the transgene of interest.

Single Cell Cloning of Transduced iPSC Population:

To obtain a single clonal population of cells, single cell plating was performed on the antibiotic resistant iPSC populations. Briefly, the antibiotic selected population of cells was diluted to achieve a cell density of 1 cell/100 μL in plating media. Cell suspension (100 μL) was plated into individual wells of a 96-well plate and grown for 3-9 days without media exchange. Following this, media was changed every second day and wells were examined for cell growth.

Cells derived from single cell clones were analyzed for pluripotency markers SSEA-5 and REA by flow cytometry and examined for protein expression of the transgene of interest (HGF or IL-12) by enzyme linked immunosorbent assay (ELISA).

Differentiation and Bioreactor-Based PLC Production:

Clones with high expression were selected for expansion and differentiation to MLC using a standardized protocol developed at PlateletBio allowing for aggregation, Stage 1, Stage 2 and Stage 3 differentiations (can provide details of differentiation protocol if needed).

For generation of ePLC (engineered PLC), eMLC (HGF or IL-12) were seeded into a microfluidic-based bioreactor and subjected to shear flow in media. The resulting ePLC were collected, concentrated by centrifugation or tangential flow filtration and subjected to further protein analysis by ELISA and/or activity assay.

Protein Characterization:

ELISAs were run according to the manufacturer recommendations using cells previously lysed in radioimmunoprecipitation assay (RIPA) buffer. The human Quantikine HGF ELISA (R&D Systems) was used to quantify HGF protein. The human p70 (IL12-A & B subunits) DuoSet ELISA (R&D Systems) was used to quantify IL-12 protein.

Activity of HGF was measuring using the STAT3 Leeporter Luciferase Reporter-HEK293 Cell Line (Abeomics, Inc.) wherein STAT3 signaling drives luciferase expression. Briefly, media alone or cell culture supernatants was added to STAT3 Leeporter-HEK293 cells grown in a 96-well microplate and incubated at 37° C. for 16 hours. Fifty μL of luciferase assay reagent (Abeomics, Inc.) was added to each well and the plate was read in a microplate reader after a period of five minutes.

From the foregoing description, it will be apparent that variations and modifications may be made to the embodiments of the present disclosure to adopt it to various usages and conditions. Such embodiments are also within the scope of the following claims. The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method of treating a subject comprising the steps of: a) inducing progenitor cells to produce megakaryocytes (MKs); b) culturing the MKs in a device or a system that supports a biologically active environment for a sufficient time period, under conditions permissible for an admixture of platelet like cells (PLCs) and extracellular vesicles production; c) collecting the PLCs and extracellular vesicles produced by the MKs; and d) administering the PLCs and/or extracellular vesicles to the subject, wherein the subject has a disorder or a disease that benefits from a treatment with the PLCs, extracellular vesicles or a combination thereof.

2. The method of claim 1 wherein the device is a bioreactor.

3. The method of claim 1 wherein the disease or disorder is selected from one or more of an immunoinflammatory disorder, a metabolic disorder, neoplastic disorder, autoimmune disorder, viral or bacterial-induced disease or infection.

4. The method of claim 1 wherein the progenitor cells are selected from one or more of megakaryocytic progenitors, megakaryocytes, proplatelets, preplatelets derived from induced pluripotent stem cells (iPSCs).

5. The method of claim 1 further comprising administering one or more therapeutic agents to the subject.

6. The method of claim 1, wherein the progenitor cells are genetically engineered to express one or more exogenous nucleic acids in one or more expression vectors encoding for one or more therapeutic proteins or polypeptides and wherein the PLCs and/or microvesicles express the one or more exogenous nucleic acids.

7. A method of treating a subject comprising the steps of: a) inducing a progenitor cell to produce megakaryocytes (MKs); b) culturing the MKs in a bioreactor for a sufficient time period, under conditions permissible for PLC and extracellular vesicles production; c) isolating and purifying the extracellular vesicles from the PLC produced by the MKs; and d) administering the extracellular vesicles to the subject, wherein the subject is suffering from one or more of immunoinflammatory disorder, a metabolic disorder, neoplastic disorder, autoimmune disorder, a viral or a bacterial-induced disease or infection.

8. The method of claim 7, wherein the extracellular vesicles comprise microvesicles and exosomes and wherein the microvesicles and exosomes are concentrated by ultracentrifugation; column chromatography; size exclusion; or filtration through a device containing an affinity matrix selective towards microvesicles or exosomes.

9. The method of claim 7, wherein the extracellular vesicles are transfected or transduced with a genetic material, and wherein the genetic material is delivered into a cell.

10. A population of cells comprising anucleated cells possessing the following characteristics: i) is derived from reprogramming of a somatic cell, progenitor cell or stem cell, the products of which are passaged ex-vivo and/or in-vitro ii) is not a cancerous cell; iii) does not exhibit uncontrolled growth or tumor formation in vivo; and iv) optionally can be locally or systemically administered or has an ability to migrate from a first position to a second position.

11. The population of cells of claim 10, wherein the somatic cell, progenitor cell or stem cell are genetically engineered to express one or more exogenous nucleic acids in one or more expression vectors encoding for one or more therapeutic proteins or polypeptides and wherein the anucleated cells express the one or more exogenous nucleic acids.

12. A pharmaceutical composition comprising the population of cells of claim 10 and a pharmaceutically acceptable agent.

13. The pharmaceutical composition of claim 12 further comprising extracellular vesicles (EVs).

14. The pharmaceutical composition of claim 12 further comprising one or more of a therapeutic agent.

15. The pharmaceutical composition of claim 12, wherein the population of cells are produced for a treatment of a disease or disorder selected from one or more of an immunoinflammatory disorder, a metabolic disorder, neoplastic disorder, autoimmune disorder, viral or bacterial-induced disease or infection.

16. A platelet like cell (PLC), variant of a reference resting stage bone marrow derived platelet cell, having a cellular structure CD63>average 2% in a comparable resting stage.

17. The PLC of claim 16, wherein the PLC is generated from a progenitor cell which upon differentiation produce an intermediate cell and the intermediate cell is passaged through a device or a system that supports a biologically active environment for a sufficient period of time to produce the PLC.

18. The PLC of claim 17 wherein the intermediate cell comprises megakaryocyte.

19. The PLC of claim 17, wherein the device is a bioreactor.

20. A pharmaceutical composition comprising the PLC of claim 16, or a pharmaceutically acceptable salt thereof, and a pharmaceutically acceptable carrier, diluent, or excipient.

21. The pharmaceutical composition of claim 20 further comprising extracellular vesicles (EVs) comprising microvesicles or exosomes or a combination thereof.

22. The pharmaceutical composition of claim 20 further comprising one or more therapeutic agents.

23. The pharmaceutical composition of claim 22 wherein the one or more therapeutic agents are selected from one or more of an antibody, a nucleic acid, a protein or a polypeptide, or a drug or a prodrug and a combination thereof.

24. A method of treating a disease or a disorder in a human patient comprising administering to the human patient an effective amount of the PLC of claim 16.

25. The method of claim 24 wherein the disease or disorder is selected from one or more of an immunoinflammatory disorder, a metabolic disorder, a neoplastic disorder, an autoimmune disorder, viral or bacterial-induced disorder.

26. A genetically engineered PLC comprising a PLC of claim 16, genetically engineered to express one or more exogenous nucleic acids in one or more expression vectors encoding for one or more proteins or polypeptides.

27. The genetically engineered PLC of claim 26 wherein the one or more exogenous nucleic acids encode for at lest one of one or more of therapeutic proteins or one or more polypeptides.

28. The genetically engineered PLC of claim 27 wherein the one or more exogenous nucleic acids are selected from one or more of siRNA, shRNA, ceDNA, DNA or RNA and a combination thereof.

29. The genetically engineered PLC of claim 27 wherein the one or more proteins or the one or more polypeptides are selected from one or more of an antibody or a fragment thereof, a growth factor, a hormone, an antigen, a cytokine and a combination thereof.

30. The genetically engineered PLC of claim 26, wherein a progenitor cell producing the PLC is genetically engineered to express one or more exogenous nucleic acids in one or more expression vectors encoding for one or more therapeutic proteins or polypeptides and wherein the PLC produced by the progenitor cell expresses the one or more exogenous nucleic acids.
